(19) United States
(12) Patent Application Publication
(10) Pub. No.: US 2011/0053901 A1
(43) Pub. Date: Mar. 3, 2011

(54) ACETYL MIMIC COMPOUNDS FOR THE INHIBITION OF ISOPRENYL-S-CYSTEINYL METHYLTRANSFERASE

(75) Inventors: Seung-Yub Lee, Princeton, NJ (US); Michael Voronkov, Pennington, NJ (US); Peter Wolanin, Princeton, NJ (US)

(73) Assignees: SIGNUM BIOSCIENCES, INC., Monmouth Junction, NJ (US); Signum Biosciences, Inc., Monmouth Junction, NJ (US)

(21) Appl. No.: 12/867,796

(22) PCT Filed: Feb. 13, 2009
(86) PCT No.: PCT/US09/34120

§ 371 Date: Nov. 16, 2010

Related U.S. Application Data
(60) Provisional application No. 61/065,939, filed on Feb. 14, 2008.

(51) Int. Cl. A61K 31/5375; C07D 211/72; A61K 31/44; C07C 279/02; A61K 31/22; C07D 257/04; A61K 31/41; A61K 31/155; C07C 257/00; A61K 31/18; C07C 311/01; A61K 31/4164; C07D 233/00; C07C 321/02; A61K 31/195; C07D 265/30; A61K 31/445; C07D 211/60; C07D 233/38; C07D 239/24; A61K 31/505; A61K 31/56; A61K 31/40

(52) U.S. Cl. 514/171; 546/312; 514/352; 560/155; 514/550; 548/252; 514/381; 514/631; 564/225; 564/95; 514/601; 514/398; 548/331.5; 562/556; 514/562; 514/237.5; 544/159; 514/315; 546/245; 514/392; 548/323.1; 544/323; 514/275; 544/332; 514/423

(57) ABSTRACT

Among other things, the present invention provides novel compounds capable of effectively inhibiting inflammatory responses that are mediated by G-proteins or GPCRs in neutrophils, macrophages and platelets. In particular, compounds of the present invention act as inhibitors of edema, inhibitors of erythema and inhibitors of MPO (myeloperoxidase), pharmaceutical compositions containing the same compounds and the use thereof for the treatment of diseases that may benefit from edema, erythema and MPO inhibition, such as inflammation (acute or chronic), asthma, autoimmune diseases, and chronic obstructive pulmonary disease (COPD) (e.g., emphysema, chronic bronchitis and small airways disease, etc.), inflammatory responses of the immune system, skin diseases (e.g., reducing acute skin irritation for patients suffering from rosacea, atopic dermatitis, seborrheic dermatitis, psoriasis), irritable bowel syndrome (e.g., Chron's disease and ulcerative colitis, etc.), and central nervous system disorders (e.g., Parkinson's disease).

### RELATED APPLICATIONS

This application claims priority to U.S. provisional patent application Ser. No. 61/065,939, filed Feb. 14, 2008, the entire disclosure of which is incorporated herein by reference.

### BACKGROUND

Inflammation often is a bodily response to infection or injury in which cells involved in detoxification and repair are mobilized to the compromised site by inflammatory mediators. The infection or injury can be a result of acute or chronic disease, disorders, conditions or trauma, environmental conditions, or aging. Examples include diseases, disorders, syndromes, conditions and injuries of the cardiovascular, digestive, integumentary, muscular, nervous, reproductive, respiratory and urinary systems, as well as, diseases, disorders, syndromes, conditions and injuries of tissue and cartilage such as atherosclerosis, irritable bowel syndrome, psoriasis, tendonitis, Alzheimer's disease and vascular dementia, multiple sclerosis, diabetes, endometriosis, asthma and kidney failure.

Treatment of inflammatory diseases or disorders with traditional anti-inflammatory drugs, e.g., corticosteroids and non-steroidal anti-inflammatory drugs (“NSAIDS”) can cause multiple side effects, e.g., appetite and weight gain, excess sweating, high blood pressure, nausea, vomiting, diarrhea, etc.

Inflammation often is characterized by a strong infiltration of polymorphonuclear leukocytes at the site of inflammation, particularly neutrophils. These cells promote tissue damage by releasing toxic substances at the vascular wall or in uninjured tissue. Neutrophil infiltration results from amplifying cascades of cell-cell communication involving signal transduction proteins, such as G-proteins, that can facilitate intracellular regulation and intercellular communication by interacting with a wide range of different regulatory receptor-transducer proteins, such as membrane bound receptors. For these interactions to occur, many of the signal transduction proteins, including virtually all G-proteins, first must be modified by the post-translational addition of a C₁₅ farnesyl or a C₂₀ geranylgeranyl polyisoprenoid group in thioether linkage to a cysteine residue located at or near the carboxyl terminus within a so-called CAAX box or related cysteine-containing sequence.

Carboxy-terminal polyisoprenoid cysteines that ultimately result from these modifications may be subject to methylesterification by a specific membrane associated S-adenosylmethionine-dependent isoprenyl-S-cysteinyl methyltransferase. Compounds that can inhibit these enzymatic reactions or otherwise alter the interactions among polyisoprenylated signal transduction proteins, such as G-proteins and the protein regulatory targets with which they interact, or other intracellular signaling proteins, may be used to mitigate leukocyte responses and, theoretically, to treat inflammatory-related conditions. (See e.g., Volker, et al., Methods Enzymol, 1995, 250: 216-225).

One signal transduction modulator compound is N-acetyl-S-farnesyl-L-cysteine (“AFC”), also referred to as N-acetyl-S-trans, trans-farnesyl-L-cysteine. AFC has been shown to be a competitive inhibitor of membrane-associated isoprenyl-S-cysteinyl methyltransferase and to block some neutrophil, macrophage, and platelet responses in vitro. Laboratory results also indicate that AFC effectively reduces dermal inflammation in mice.

Numerous drugs have been used to treat inflammation, all of which suffer from some side effects, some of which are serious. For example, common side effects of corticosteroids include increased appetite and weight gain, deposits of fat, in chest, face, upper back and stomach, water and salt retention leading to swelling and edema, high blood pressure, diabetes, excess sweating, telangiectasia (dilation of capillaries), slowed healing of wounds, osteoporosis, cataracts, acne, hirsutism, muscle weakness, atrophy of the skin and mucous membranes, an increased susceptibility to infection, and stomach ulcers.

In another example, studies have demonstrated an increased risk of cardiovascular events associated with the use of anti-inflammatory agents known as Cox-II inhibitors, such as such as Celebrex® and Vioxx® (See, e.g., Solomon et al., N. Engl. J. Med 2005; 352: 1071-80; Nussmeier et al, N. Engl. J. Med. 2005; 352: 1081-91).

NSAIDS such as aspirin and ibuprofen are also traditionally used to treat inflammation. Side effects of NSAIDS vary between drugs, but generally include nausea, vomiting, diarrhea, constipation, decreased appetite, rash, dizziness, headache, drowsiness and photosensitivity. NSAIDs also may cause fluid retention, leading to edema. The most serious side effects of NSAIDs use include kidney failure, liver failure, ulcers and prolonged bleeding after an injury or surgery. NSAIDs can produce shortness of breath in individuals allergic to them. People with asthma are at a higher risk for experiencing serious allergic reaction to NSAIDS. Individuals with a serious allergy to one NSAID are likely to experience a similar reaction to a different NSAID.

Thus, there is a need for a non-steroidal anti-inflammatory compound that lacks the side effects of corticosteroids and NSAIDS. It has been found that signal transduction modulator compounds may impede inflammation. Without being bound by any particular theory, the impediment of inflammation may be a result of the ability of signal transduction modulator compounds to alter cell to cell signaling. The present invention, therefore, is directed to novel signal transduction modulator compounds for treating and/or preventing inflammation, and for other unmet needs.

Other background and methods may be found in U.S. Pat. Nos. 5,043,268, 5,202,456, 5,705,528, and 5,705,528, as well as U.S. patent application serial numbers 2005/0277694 and 2007/0004803, each of which is incorporated herein by reference.

### SUMMARY

Among other things, the present invention provides novel compounds that modulate the G-protein signaling cascade. The present invention provides certain compounds that are structurally related to N-acetyl-S-farnesyl-L-cysteine (“AFC”).

The present invention demonstrates desirable characteristics of certain such compounds. For example, among other things, the present invention demonstrates that certain such compounds and/or compositions show inhibition of edema, erythema and dermal neutrophil infiltration, as measured by inhibition of MPO (myeloperoxidase).

In certain embodiments, compounds provided by the present invention have the structure set forth in formula I,

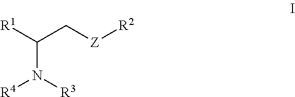

In certain embodiments, compounds provided by the present invention have the structure set forth in formula Ia,

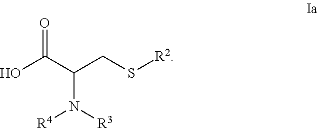

In certain embodiments, compounds of formulae I, Ia and/or Ib are provided in a pharmaceutically acceptable salt form, as enantiomers, diastereomers, double bond isomers, particular crystal forms or prodrugs thereof. Other embodiments are described in more detail below.

The present invention also provides compositions containing compounds described herein, methods of preparing such compounds and/or compositions, and methods of using such compounds and/or compositions.

In certain embodiments, the present invention provides uses of provided compounds and/or compositions in the treatment of inflammation and/or misregulation of cellular processes. In certain embodiments, the present invention provides uses of provided compounds and/or compositions in the treatment of diseases that may benefit from edema inhibition, erythema inhibition and/or MPO inhibition, such as treating or lessening the severity of inflammatory diseases or disorders selected from inflammation (acute or chronic), asthma, autoimmune diseases, and chronic obstructive pulmonary disease (COPD) (e.g., emphysema, chronic bronchitis and small airways disease, etc.), inflammatory responses of the immune system, skin diseases (e.g., reducing acute skin irritation for patients suffering from rosacea, atopic dermatitis, seborrheic dermatitis, psoriasis), irritable bowel syndrome (e.g., Chron's disease and ulcerative colitis, etc.), and central nervous system disorders (e.g., Parkinson's disease).

While various aspects of the disclosure herein are illustrated through the use of certain compounds, it is an object of the present invention to extend the various embodiments described herein utilizing a compound or a composition comprising a compound of formula I. Various other compounds and/or compositions as described herein would be known to those skilled in the art made aware of this disclosure. As described more fully below, in the accompanying figures, examples and descriptions, a related object of this disclosure includes the provision of various compounds and/or compositions that can be used in any of a variety of applications.

### DEFINITIONS

“Acyl group”: As used herein, the term “acyl group” includes a group —R—C(═O)—, where R is an organic group, for example but not limited to, an alkyl group. An example may be the acetyl group —CH₃—C(═O)—, referred to herein as “Ac”.

“Aliphatic group”: As used herein, the term “aliphatic group” means a hydrocarbon group, but not limited to, straight or branched chain hydrocarbon chains, such as straight or branched chain alkanes, straight or branched chain alkenes with one or more double bonds, and straight or branched chain allynes with one or more triple bonds and optionally also with one or more double bonds, for example. An aliphatic group may optionally be substituted with one or more suitable substituents. The term “aliphatic” is used interchangeably with the term “aliphatic group” herein. In some embodiments, an aliphatic group is a straight or branched chain alkyl group, with about 10 to about 25 carbon atoms or a straight or branched chain alkenyl group, with about 10 to about 25 carbon atoms and one or more double bonds. In certain embodiments, preferred aliphatic groups may include all stereoisomers and double bond isomers of farnesyl or geranylgeranyl, unsubstituted or substituted with one or more suitable substituents. In certain embodiments, an “aliphatic” group may be cyclic.

“Alkenyl group”: As used herein, the term “alkenyl group” means a monovalent, unbranched or branched hydrocarbon chain having one or more double bonds therein. The double bond of an alkenyl group can be unconjugated or conjugated to another unsaturated group. Suitable alkenyl groups may include, but are not limited to (C₂-C₆) alkenyl groups, such as vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl. An alkenyl group may be unsubstituted or optionally substituted with one or two suitable substituents. In certain embodiments, an “alkenyl” group may be cyclic. The term “alkenyl” is used interchangeably with the term “alkenyl group” herein.

“Alkoxy group”: As used herein, the term “alkoxy group” means an —O-alkyl group, where alkyl is as defined above. An alkoxy group may be unsubstituted or optionally substituted with one or more suitable substituents. The term “alkoxy” is used interchangeably with the term “alkoxy group” herein.

“Alkoxycarbonyl group”: As used herein, the term “alkoxycarbonyl group” means a monovalent group of the formula —C(═O)—O-alkyl. Preferably, the alkyl group of an alkoxycarbonyl group is from 1 to 8 carbon atoms in length, referred to herein as a “lower alkoxycarbonyl group.”

“Alkyl group”: As used herein, the term “alkyl group” means a saturated, monovalent, unbranched or branched hydrocarbon chain. Examples of alkyl groups include, but are not limited to, (C₁-C₆) alkyl groups, such as methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, and hexyl, and longer alkyl groups, such as heptyl, and octyl. An alkyl group can be unsubstituted or optionally substituted with one or two suitable substituents. In certain embodiments, an “alkyl” group may be cyclic. The term “alkyl” is used interchangeably with the term “alkyl group” herein.

“Alkynyl group”: As used herein, the term “alkynyl group” means monovalent, unbranched or branched hydrocarbon chain having one or more triple bonds therein. The triple bond of an alkynyl group may be unconjugated or conjugated to another unsaturated group. Suitable alkynyl groups may include, but are not limited to, —(C₂-C₆)alkynyl groups, such as ethynyl, propynyl, butynyl, pentynyl, hexynyl, methylpropynyl, 4-methyl-1-butynyl, 4-propyl-2-pentynyl, and 4-butyl-2-hexynyl. An alkynyl group may be unsubstituted or optionally substituted with one or two suitable substituents. The term “alkynyl” is used interchangeably with the term “alkynyl group” herein.

“Amide”: An “amide” includes compounds that have a trivalent nitrogen attached to a carbonyl group —(C(═O)—NH₂), such as for example methylamide, ethylamide, propylamide, and the like.

“Animal”: The term animal, as used herein, refers to humans as well as non-human animals, including, for example, mammals, birds, reptiles, amphibians, and fish. Preferably, the non-human animal is a mammal (e.g., a rodent, a mouse, a rat, a rabbit, a monkey, a dog, a cat, a primate, or a pig). A non-human animal may be a transgenic animal.

“Aryl group”: As used herein, the term “aryl group” means a monocyclic or polycyclic-aromatic radical having carbon and hydrogen atoms. Examples of suitable aryl groups may include, but are not limited to, phenyl, tolyl, anthacenyl, fluorenyl, indenyl, azulenyl, naphthyl, 1-naphthyl, 2-naphthyl, and biphenyl as well as benzo-fused carbocyclic moieties such as 5, 6, 7, 8-tetrahydronaphthyl. An aryl group can be unsubstituted or optionally substituted with one or two suitable substituents as defined below. An aryl group optionally may be fused to a cycloalkyl group, fused to another aryl group, fused to a heteroaryl group, or fused to a heterocycloalkyl group. Preferred aryl groups may include, but are not limited to, monocyclic or bicyclic aromatic hydrocarbon radicals of 6 to 12 ring atoms, and optionally substituted independently with one or more suitable substituents. The term “aryl” is used interchangeably with the term “aryl group” herein.

“Aryloxy group”: As used herein, the term “aryloxy group” means an —O-aryl group, wherein aryl is as defined above. An aryloxy group may be unsubstituted or optionally substituted with one or more suitable substituents. The term “aryloxy” is used interchangeably with the term “aryloxy group” herein.

“Associated with”: When two entities are “associated with” one another as described herein, they are linked by a direct or indirect covalent or non-covalent interaction. Preferably, the association is covalent. Desirable non-covalent interactions include hydrogen bonding, van der Waals interactions, hydrophobic interactions, magnetic interactions, electrostatic interactions, etc.

“Carbamoyl group”: As used herein, the term “carbamoyl group” means the radical —C(═O)N(R′)₂, where R′ is chosen from the group consisting of hydrogen, alkyl, and aryl. The term “carbamoyl” is used interchangeably with the term “carbamoyl group” herein.

“Carbonyl group”: As used herein, a “carbonyl group” is a divalent group of the formula —C(═O). The term “carbonyl” is used interchangeably with the term “carbonyl group” herein.

“Composition”: The term “composition”, as in pharmaceutical composition, encompasses a product with active ingredient(s) and a carrier with one or more inert ingredient(s). Accordingly, pharmaceutical compositions may encompass a composition with a compound and a pharmaceutically acceptable carrier. In general, pharmaceutical compositions are prepared and formulated for administration to animals, e.g., humans. Pharmaceutical compositions therefore are not present in toxic levels and/or do not have toxic substances. In certain embodiments, compositions of the present invention are those that are, within the scope of sound medical judgment, suitable for use in contact with the tissues of humans and lower animals with undue toxicity, irritation, allergic response, and the like, commensurate with a reasonable benefit/risk ratio.

“Cyclic radical”: As used herein, the term “cyclic radical” means an aryl group, a cycloalkyl group, a heterocycloalkyl group or a heteroaryl group.

“Cycloalkyl group”: As used herein, the term “cycloalkyl group” means a monocyclic or polycyclic saturated ring with carbon and hydrogen atoms and having no carbon—carbon multiple bonds. Examples of cycloalkyl groups may include, but are not limited to, (C₃-C₇)cycloallyl groups, such as cyclopropyl, cyclobutyl, cyclopentyl, cyclohexyl, and cycloheptyl, and saturated cyclic and bicyclic terpenes. A cycloalkyl group may be unsubstituted or optionally substituted with one or two suitable substituents as defined below. A cycloalkyl group optionally may be fused to another cycloalkyl group, fused to an aryl group, fused to a heteroaryl group, or fused to a heterocycloalkyl group. The term “cycloalkyl” is used interchangeably with the term “cycloalkyl group” herein.

“G-protein mediated condition”: The term “G-protein mediated condition”, as used herein means any disease or other deleterious condition for which the appearance, incidence, and/or severity of one or more symptoms correlates with changes in a G-protein signaling cascade. In some embodiments, one or more symptoms of the disease or condition is caused by a defect or alteration in G-protein signaling.

“Halogen”: As used herein, the term “halogen” means fluorine, chlorine, bromine, or iodine. Correspondingly, the meaning of the terms “halo” and “Hal” encompass fluoro, chloro, bromo, and iodo.

“Heteroaryl group”: As used herein, the term “heteroaryl group” means a monocyclic- or polycyclic aromatic ring comprising carbon atoms, hydrogen atoms, and one or more heteroatoms, preferably 1 to 4 heteroatoms, independently selected from nitrogen, oxygen, and sulfur groups may include, but are not limited to, pyrazolyl, triazolyl, tetrazolyl, pyridyl, pyridazinyl, pyrazyl, indolyl, triazinyl, pyrrolyl, pyrazolyl, imidazolyl, (1,2,3)-triazolyl, (1,2,4)-triazolyl, pyrazinyl, pyrimidinyl, tetrazolyl, furyl, thienyl, 4H-1,4-thiazine, isoxazolyl, thiazolyl, phienyl, isoxazolyl, oxazolyl, pyrazolyl, tetrazolyl, triazolyl, oxadiazolyl, thiadiazolyl, isoxazolyl, triazinyl, and pyrazinyl. Bicyclic heteroaromatic rings may include, but are not limited to, benzothiadiazolyl, indolyl, benzothiophenyl, benzofuryl, benzimidazolyl, purinyl, benzisoxazolyl, benzothiazolyl, quinolinyl, benzotriazolyl, benzoxazolyl, isoquinolinyl, purinyl, furopyridinyl and thienopyridinyl. A heteroaryl can be unsubstituted or optionally substituted with one or more suitable substituents as defined below. A heteroaryl group optionally may be fused to another heteroaryl group, fused to an aryl group, fused to a cycloalkyl group, or fused to a heterocycloalkyl group. The term “heteroaryl” is used interchangeably with the term “heteroaryl group” herein.

“Heterocyclic radical” or “heterocyclic ring”: As used herein, the terms “heterocyclic radical” or “heterocyclic ring” mean a heterocycloalkyl group or a heteroaryl group.

“Heterocycloalkyl group”: As used herein, the term “heterocycloalkyl group” means a monocyclic or polycyclic ring with carbon and hydrogen atoms and at least one heteroatom, (in some embodiments, 1 to 3 heteroatoms), for example selected from nitrogen, oxygen, and sulfur. A heterocycloalkyl group may be fused to an aryl or heteroaryl group. Examples of heterocycloalkyl groups may include, but are not limited to, pyrrolidinyl, pyrrolidino, piperidinyl, piperidino, piperazinyl, piperazino, morpholinyl, morpholino, thiomorpholinyl, thiomorpholino, and pyranyl. A heterocycloalkyl group may be unsubstituted or optionally substituted with one or more suitable substituents as defined below. A heterocycloalkyl group optionally may be fused to a cycloalkyl group, fused to an aryl group, fused to a heteroaryl group, or fused to another heterocycloalkyl group. The term “heterocycloalkyl” is used interchangeably with the term “heterocycloalkyl group” herein.

“In combination”: As used herein, the phrase “in combination” refers to simultaneous administration of two or more agents to a subject. It will be appreciated that two or more agents are considered to be administered “in combination” whenever a subject is simultaneously exposed to both (or all) of the agents. Each of the two or more agents may be administered according to a different schedule; it is not required that individual doses of different agents be administered at the same time, or in the same composition. Rather, so long as both (or more) agents remain in the subject's body at the same time they may be considered to be administered “in combination”.

“Independently selected”: The term “independently selected” is used herein to indicate that individual R groups can be identical or different.

“Modulate”: To “modulate” a parameter is to change (i.e., increase or decrease) level and/or activity of the parameter (e.g., an increase or decrease in binding, an increase or decrease in activity, etc.).

“Modulator”: The term “modulator” refers to an agent whose presence or whose level results in a change (i.e., increase or decrease in level and/or activity of its target, e.g., in the GPCR signal transduction pathway. In some embodiments, a modulator alters interaction between a protein in the GPCR signal transduction pathway and one or more other entities. In some embodiments, a modulator alters interaction between a protein in the GPCR signal transduction pathway and a substrate. Determination of whether an agent is a modulator can be performed directly or indirectly. For example, determination of whether an agent modulates an interaction can be performed directly, e.g., using an assay that detects the interaction between a protein in the GPCR signal transduction pathway and a substrate. Alternatively or additionally, determination of whether an agent modulates an interaction can be performed with a technique that indirectly detects modulation, e.g., a technique that detects a biological activity that is downstream of, and dependent on, the protein-substrate interaction.

“Oxo group”: As used herein, an “oxo group” is a group of the formula (═O).

“Pharmaceutically acceptable ester”: The term “pharmaceutically acceptable ester” refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic, and alkanedioic acids, in which each alkyl or alkenyl group advantageously has not more than 6 carbon atoms. Examples of particular esters include formates, acetates, propionates, butyrates, acrylates, and ethylsuccinates. In certain embodiments, the esters are cleaved by enzymes such as esterases.

“Pharmaceutically acceptable prodrugs”: The term “pharmaceutically acceptable prodrugs” as used herein refers to those prodrugs of compounds of the present invention which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of compounds of the invention. The term “prodrug” refers to compounds that are rapidly transformed in vivo to yield the parent compound of the above formula, for example by hydrolysis in blood. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference.

“Pharmaceutically acceptable salt(s)”: The term “pharmaceutically acceptable salt(s)”, as used herein may include but is not limited to salts of acidic or basic groups that may be present in compounds of the present invention. Compounds that are basic in nature may be capable of forming a wide variety of salts with various inorganic and organic acids. Such non-toxic salts, i.e., salts containing pharmacologically acceptable anions, may include but are not limited to hydrochloride, hydrobromide, hydroiodide, nitrate, sulfate, bisulfate, phosphate, acid phosphate, isonicotinate, acetate, lactate, salicylate, citrate, acid citrate, tartrate, oxalate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, ptoluenesulfonate and pamoate (i.e., 1,1′-methylene-bis-(2-hydroxy-3-naphthoate)) salts. Compounds of the present invention that may include an amino group also may form pharmaceutically acceptable salts with various amino acids, in addition to the acids mentioned above. Compounds that may be acidic in nature are capable of forming base salts with various pharmacologically acceptable cations. Examples of such salts may include alkali metal or alkaline earth metal salts and, particularly, calcium, magnesium, sodium lithium, zinc, potassium, and iron salts. Other such salts may include pharmaceutically acceptable organic bases such as ammonia, arginine, benethamine, benzathine, deanol, diethanolamine, diethylamine, -2-diethylaminoethanol, ethanolamine, ethylenediamine, lysine, -2-hydroxyethylmorpholine, piperazine, -2-hydroxyethylpyrrolidine, triethanolamine, tromethamine.

“Prophylactically effective”, “preventing” or “preventive”: As used herein, the term “prophylactically effective” or “preventive” means that administration prior to the onset of symptoms or the features of a disease, disorder or condition, delays onset of and/or reduces severity of one or more such symptoms or other features.

“Room temperature”: As used herein, the term “room temperature” or “RT” means a temperature within the range of about 17° C. to about 26° C. In some embodiments, room temperature is within the range of about 23° C. to about 26° C. In certain embodiments, room temperature is within the range of about 24° C. to about 25° C.

“Skin Irritant”: A “skin irritant” is an agent that, when applied to skin or a skin equivalents, elicits a cellular response characterized by expression of an “irritant responsive gene” and/or by other indications of irritation (e.g., redness, inflammation, etc.). Examples of known skin irritants include, but are not limited to, sodium dodecyl sulfate (“SDS”), calcipotriol, and trans-retinoic acid. The term “skin irritant” is also intended to encompass unknown or suspected irritants, including but not limited to, those containing in some pharmaceuticals, cosmetics, and consumer products.

“Small Molecule”: As used herein, the term “small molecule” refers to an organic compound that is characterized in that it contains several carbon-carbon bonds, and typically has a molecular weight of less than about 1500. Small molecules may be natural products (i.e., found in nature, or may be non-natural compounds. Small molecules may be prepared by isolation (e.g., if natural products) and/or synthesized in the laboratory.

“Suitable substituent”: As used herein, the term “suitable substituent” refers to a group that does not nullify the therapeutic or pharmaceutical utility of compounds of the present invention or the synthetic utility of the intermediates useful for preparing them. Examples of suitable substituents may include, but are not limited to: alkyl; alkenyl; alkynyl; aryl; heteroaryl; heterocycloalkyl; cycloalkyl; —O-alkyl, —O-alkenyl, —O-alkynyl, —O-aryl, —CN, —OH, oxo, halo, —C(═O)OH, —C(═O)halo, —OC(═O)halo, —CF₃, N₃, NO₂, —NH₂, —NH(alkyl), —N(alkyl)₂, NH(aryl), —N(aryl)₂, —C(═O)NH₂, —C(═O)H(alkyl), —C(═O)N(alkyl)₂, —C(═O)NH(aryl), —C(═O)N(aryl)₂, —OC(═O)NH₂, —C(═O)NH(heteroaryl), —C(═O)N(heteroaryl)₂, —NHOH, —NOH(alkyl), —NOH(aryl), —OC(═O)H(alkyl), —OC(═O)N(alkyl)₂, —OC(═O)NH(aryl), —OC(═O)N(aryl)₂, —CHO, —C(═O)(alkyl), —C(═O)(aryl), —C(═O)O(alkyl), —C(═O)O(aryl), —OC(═O)(alkyl), —OC(═O)(aryl), —OC(═O)O(alkyl), —OC(═O)O(aryl), —S-alkyl, —S-alkenyl, —S-alkynyl, —O—S(═O)₂-alkenyl, —O—S(═O)₂-alkynyl, —O—S(═O)₂-aryl, —(CH₂), —NH₂, —(CH₂)ₙ—NH(alkyl), —(CH₂)ₙ—N(alkyl)₂, —(CH₂)ₙ—NH(aryl), or —(CH₂)ₙN(aryl)₂, wherein n is 1 to 8. In certain embodiments, a “suitable substituent” is used in cases allowing for “optional substitution” or allowing for an “optionally substituted” moiety.

Examples of suitable substituents may include, but are not limited to: —(C₁-C₈)alkyl, —(C₁-C₈)alkenyl, —(C₁-C₈)alkynyl, phenyl, —(C₂-C₅) heteroaryl, —(C₁-C₆)heterocycloalkyl, —(C₃-C₇)cycloalkyl, —O—(C₁-C₈)alkyl, —O—(C₁-C₈)alkenyl, —O—(C₁-C₈)alkynyl, —O-phenyl, —CN, —OH, oxo, halo, —C(═O)OH, —COhalo, —OC(═O)halo, —CF₃, N₃, NO₂, —NH₂, —NH((C₁-C₈)alkyl), —N((C₁-C₈)alkyl)₂, —NH(phenyl), —N(phenyl)₂, —C(═O)NH₂, —C(═O)NH((C₁-C₈)alkyl), —C(═O)N((C₁-C₈)alkyl)₂, —C(═O)NH(phenyl), —C(═O)N(phenyl)₂, —OC(═O)NH₂, —NHOH, —NOH((C₁-C₈)alkyl), —NOH(phenyl), —OC(═O)NH((C₁-C₈)alkyl), —OC(═O)N((C₁-C₈)alkyl)₂, —OC(═O)NH(phenyl), —OC(═O)N(phenyl)₂, —CHO, —CO((C₁-C₈)alkyl), —CO(phenyl), —C(═O)O((C₁-C₈)alkyl), —C(═O)O(phenyl), —OC(═O)((C₁-C₈)alkyl), —OC(═O)(phenyl), —OC(═O)O((C₁-C₈)alkyl), —OC(═O)O(phenyl), —S—(C₁-C₈)alkyl, —S—(C₁-C₈)alkenyl, —S—(C₁-C₈)alkynyl, —S-phenyl, —NHS(O)₂-phenyl, —NHS(O)₂-alkyl, —NHS(O)₂—(C₁-C₈)alkenyl, —NHS(O)₂—(C₁-C₈)alkynyl, —NHS(O)₂, —SC(O)-phenyl, —SC(O)-alkyl, —SC(O)—(C₁-C₈)alkenyl, —SC(O)—(C₁-C₈ alkynyl), —O—S(═O)₂—(C₁-C₈)alkyl, —O—S(═O)₂—(C₁-C₈)alkenyl, —O—S(═O)₂—(C₁-C₈)alkynyl, —O—S(═O)₂-phenyl, —(CH₂)ₙNH₂, —(CH₂)ₙNH((C₁-C₈)alkyl), —(CH₂)ₙN((C₁-C₈)alkyl)₂, —(CH₂)ₙNH(aryl), or —(CH₂)ₙN(phenyl)₂, wherein n is 1 to 8. One of ordinary skill in the art may readily choose a suitable substituent based on the stability and pharmacological and synthetic activity of compounds of the present invention. A designation of (Cₓ-C_{y}) indicates the number of carbon atoms in the group, for example, (C₁-C₈) means that the group contains 1 to 8 carbon atoms.

“Synthon”: As used herein, the term “synthon” refers to a structural unit within a small molecule that can be formed and/or assembled by synthetic procedures known to one of ordinary skill in the art.

“Therapeutically effective amount”: As used herein, the term “therapeutically effective amount” means an amount of a compound that may elicit a biological or medical response in a mammal that is being that is being treated by a medical doctor or other clinician. The therapeutic effect may be objective (i.e., measurable by some test or marker) or subjective (i.e., subject gives an indication of or feels an effect). A therapeutically effective amount is commonly administered in a dosing regimen that may comprise multiple unit doses.

“Treat” “treating” and “treatment”: The terms “treat” “treating” and “treatment,” as used herein, contemplate an action that occurs while a patient is suffering from a specified disease, disorder, or condition that delays onset and/or reduces the severity of and/or the frequency of one or more symptoms or features of a disease, disorder, or condition.

“Wavy line” ( [symbol] ): As used in the chemical structure drawings, the “wavy line” is used in one or two different contexts. In certain embodiments, a “wavy line” indicates a point of attachment of a particular chemical group to another chemical group within a molecule, and beyond the depiction of that wavy line, the remainder of the molecule is not shown in the image. In certain embodiments, a “wavy line,” when adjacent to a double bond, indicates that that double bond can be in either the E or Z configuration (i.e., the compound may be a double bond isomer).

Unit dosage form: The expression “unit dosage form” as used herein refers to a physically discrete unit of a provided formulation appropriate for the subject to be treated. It will be understood, however, that the total daily usage of provided formulation will be decided by the attending physician within the scope of sound medical judgment. The specific effective dose level for any particular subject or organism will depend upon a variety of factors including the disorder being treated and the severity of the disorder, activity of specific active agent employed, specific formulation employed, age, body weight, general health, sex and diet of the subject, time of administration, rate of excretion of the specific active agent employed, duration of the treatment, drugs and/or additional therapies used in combination or coincidental with specific compound(s) employed, and like factors well known in the medical arts. In certain embodiments, a unit dosage form designed for administration as part of a regimen to deliver a therapeutically effective amount is considered to contain a “therapeutically effective amount.” In certain embodiments, a unit dosage form may itself deliver a “therapeutically effective amount.”

### DETAILED DESCRIPTION OF CERTAIN EMBODIMENTS

### 1. Description of Exemplary Compounds

Compounds provided by the present invention include those described generally above, and are further illustrated by all classes, subclasses and species of each of these compounds disclosed herein.

According to one aspect, the present invention provides compound of formula I:

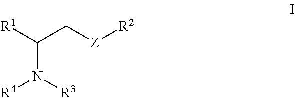

or a pharmaceutically acceptable salt, enantiomer, diastereomer, or double bond isomer thereof, wherein:

R¹ is an optionally substituted heteroaryl group or:

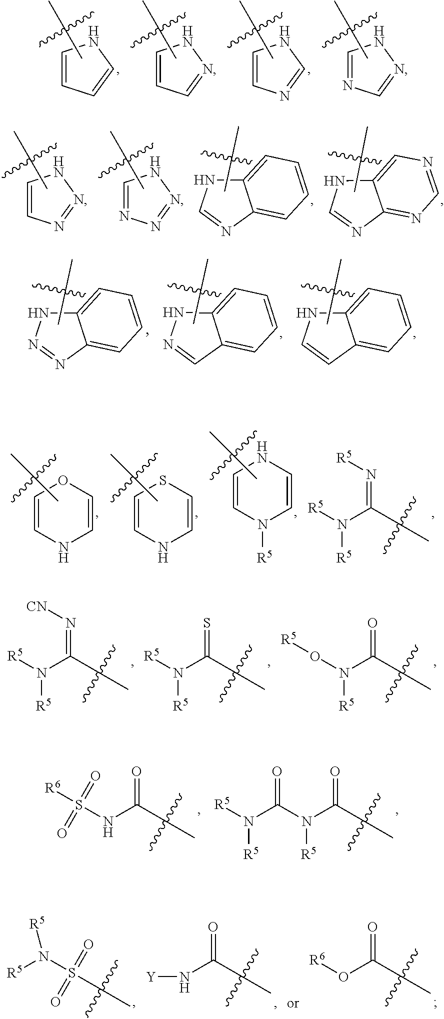

R² is an aliphatic group substituted with one or more R⁷ groups;

R³ is an optionally substituted heteroaryl group,

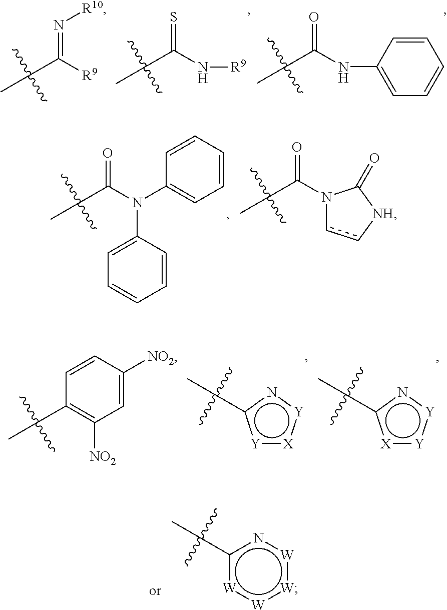

W is independently —C(R¹²)— or N;

R¹² is halo, hydrogen, CF₃, N(R⁵)₂, oxo, alkyl, alkenyl, alkynyl or aryl;

X is —O—, S, —N—, —N(R⁵)—, —C(R¹¹)— or —C(R⁶)—;

Y is independently —C(R¹¹)—, N or —OH;

R¹¹ is hydrogen, F, CH₃, CF₃, OH, —NH₂, —NHNH₂, alkyl, alkenyl, alkynyl or aryl;

R⁴ is H, alkyl, aryl, alkenyl, alkynyl, or a cyclic radical, wherein R⁴ is optionally substituted with one or two R⁷ groups;

R⁵ is independently H, alkyl, aryl, alkenyl, or alkynyl, or —C(═O)O-t-butyl wherein R⁵ is optionally substituted with one or two R⁷ groups;

R⁶ is H, alkyl, aryl, alkenyl, alkynyl, or a cyclic radical, wherein R⁶ is optionally substituted with one or two R⁷ groups;

R⁷ is —NHC(═O)(C₁-C₈)alkyl, —(C₁-C₈)alkyl, —(C₁-C₈)alkenyl, —(C₁-C₈)alkynyl, phenyl, —(C₂-C₅)heteroaryl, —(C₁-C₆)heterocycloalkyl, —(C₃-C₇)cycloalkyl, —O—(C₁-C₈)alkyl, —O—(C₁-C₈)alkenyl, —O—(C₁-C₈)alkynyl, —O-phenyl, —CN, —OH, oxo, halo, —C(═O)OH, —COhalo, —OC(═O)halo, —CF₃, N₃, NO₂, —NH₂, —NH(C₁-C₈)alkyl, —N(C₁-C₈)alkyl)₂, —NH(phenyl), —N(phenyl)₂, —C(═O)NH₂, —C(═O)NH((C₁-C₈)alkyl), —C(═O)N((C₁-C₈)alkyl)₂, —C(═O)NH(phenyl), —C(═O)N(phenyl)₂, —OC(═O)NH₂, —NHOH, —NOH((C₁-C₈)alkyl), —NOH(phenyl), —OC(═O)NH((C₁-C₈)alkyl), —OC(═O)N((C₁-C₈)alkyl)₂, —OC(═O)NH(phenyl), —OC(═O)N(phenyl)₂, —CHO, —CO((C₁-C₈)alkyl), —CO(phenyl), —C(═O)O((C₁-C₈)alkyl), —C(═O)O(phenyl), —OC(═O)((C₁-C₈)alkyl), —OC(═O)(phenyl), —OC(═O)O((C₁-C₈)alkyl), —OC(═O)O(phenyl), —S—(C₁-C₈)alkyl, —S—(C₁-C₈)alkenyl, —S—(C₁-C₈)alkynyl, and —S-phenyl, —NHS(O)₂-phenyl, —NHS(O)₂-alkyl, —NHS(O)₂—(C₁-C₈)alkenyl, —NHS(O)₂—(C₁-C₈)alkynyl, —NHS(O)₂, —SC(O)-phenyl, —SC(O)-alkyl, —SC(O)—(C₁-C₈)alkenyl, —SC(O)—(C₁-C₈ alkynyl), —O—S(═O)₂—(C₁-C₈)alkyl, —O—S(═O)₂—(C₁-C₈)alkenyl, —O—S(═O)₂—(C₁-C₈)alkynyl, —O—S(═O)₂-phenyl, —(CH₂)ₙNH₂, —(CH₂)ₙ—NH((C₁-C₈)alkyl), —(CH₂)ₙN((C₁-C₈)alkyl)₂, —(CH₂)ₙNH(phenyl), or —(CH₂)ₙN(phenyl)₂, wherein n is 1 to 8.

R⁹ is H, alkyl, alkenyl, alkynyl, aryl, —N(R⁵)₂;

R¹⁰ is H, alkyl, alkenyl, alkynyl, aryl, —CN, —S(═O)₂—R⁶ or —C(═O)O-t-butyl; and

Z is —S—, —O—, —Se—, —S(O)—, —SO₂—, or —NH—;

wherein, each of the dashed lines independently represents the presence or absence of a double bond.

In certain embodiments, the present invention provides a compound of formula I,

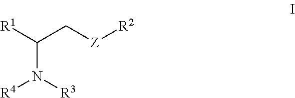

or a pharmaceutically acceptable salt, enantiomer, diastereomer, or double bond isomer thereof, wherein:

R¹ is a heteroaryl group or is selected from

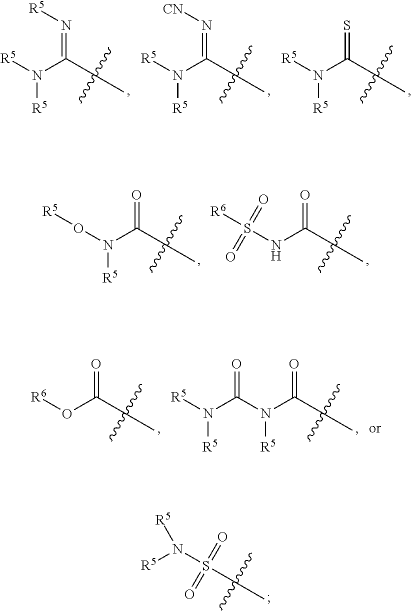

R² is an aliphatic group substituted with one or more R⁷ groups;

R³ is a heteroaryl group or

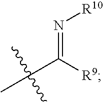

R⁴ is H, alkyl, aryl, alkenyl, alkynyl, or a cyclic radical, wherein R⁴ is optionally substituted with one or two R⁷ groups;

R⁵ is independently H, alkyl, aryl, alkenyl, or alkynyl, wherein R⁵ is optionally substituted with one or two R⁷ groups;

R⁶ is H, alkyl, aryl, alkenyl, alkynyl, or a cyclic radical, wherein R⁶ is optionally substituted with one or two R⁷ groups;

R⁷ is —NHC(═O)(C₁-C₈)alkyl, —(C₁-C₈)alkyl, —(C₁-C₈)alkenyl, —(C₁-C₈)alkynyl, phenyl, —(C₂-C₅)heteroaryl, —(C₁-C₆)heterocycloalkyl, —(C₃-C₇)cycloalkyl, —O—(C₁-C₈)alkyl, —O—(C₁-C₈)alkenyl, —O—(C₁-C₈)alkynyl, —O-phenyl, —CN, —OH, oxo, halo, —C(═O)OH, —COhalo, —OC(═O)halo, —CF₃, N₃, NO₂, —NH₂, —NH((C₁-C₈)alkyl), —N((C₁-C₈)alkyl)₂, —NH(phenyl), —N(phenyl)₂, —C(═O)NH₂, —(═O)NH((C₁-C₈)alkyl), —C(═O)N((C₁-C₈)alkyl)₂, —C(═O)NH(phenyl), —C(═O)N(phenyl)₂, —OC(═O)NH₂, —NHOH, —NOH((C₁-C₈)alkyl), —NOH(phenyl), —OC(═O)NH((C₁-C₈)alkyl), —OC(═O)N((C₁-C₈)alkyl)₂, —OC(═O)NH(phenyl), —OC(═O)N(phenyl)₂, —CHO, —CO((C₁-C₈)alkyl), —CO(phenyl), —C(═O)O((C₁-C₈)alkyl), —C(═O)O(phenyl), —OC(═O)((C₁-C₈)alkyl), —OC(═O)(phenyl), —OC(═O)O((C₁-C₈)alkyl), —OC(═O)O(phenyl), —S—(C₁-C₈)alkyl, —S—(C₁-C₈)alkenyl, —S—(C₁-C₈)alkynyl, and —S-phenyl, —NHS(O)₂-phenyl, —NHS(O)₂-alkyl, —NHS(O)₂—(C₁-C₈)alkenyl, —NHS(O)₂—(C₁-C₈)alkynyl, —NHS(O)₂, —SC(O)-phenyl, —SC(O)-alkyl, —SC(O)—(C₁-C₈)alkenyl, —SC(O)—(C₁-C₈ alkynyl), —O—S(═O)₂—(C₁-C₈)alkyl, —O—S(═O)₂—(C₁-C₈)alkenyl, —O—S(═O)₂—(C₁-C₈)alkynyl, —O—S(═O)₂-phenyl, —(CH₂)ₙNH₂, —(CH₂)ₙ—NH((C₁-C₈)alkyl), —(CH₂)ₙN((C₁-C₈)alkyl)₂, —(CH₂)ₙNH(phenyl), or —(CH₂)ₙN(phenyl)₂, wherein n is 1 to 8.

R⁹ is H, alkyl, alkenyl, alkynyl, aryl, —N(R⁵)₂;

R¹⁰ is H, alkyl, alkenyl, alkynyl, aryl, CN, —S(═O)₂—R⁶; and

Z is —S—, —O—, —Se—, —S(O)—, —SO₂—, or —NH—.

In certain embodiments, the present invention provides a compound of formula I,

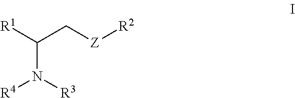

or a pharmaceutically acceptable salt, enantiomer, diastereomer, or double bond isomer thereof, wherein:

R₁ is

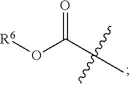

R² is a straight or branched chain aliphatic group having 10 to 25 carbon atoms and one or more double bonds;

R³ is

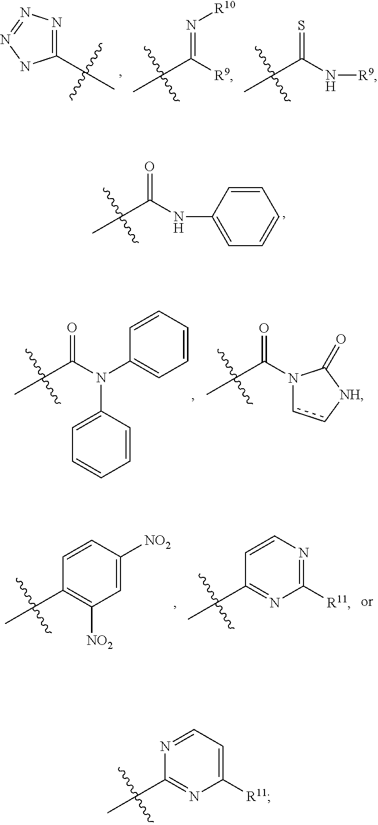

R⁴ is H;

R⁶ is H or alkyl;

R⁹ is H or alkyl;

R¹⁰ is H alkyl or CN;

R¹¹ is —NH₂, —NHNH₂ or hydrogen;

X=—N—, —C(R⁶)— or —O—; and

Z is —S— or —Se—;

wherein, each of the dashed lines independently represents the presence or absence of a double bond.

In certain embodiments, the present invention provides a compound of formula Ia,

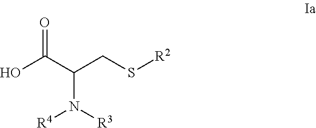

or a pharmaceutically acceptable salt, enantiomer, diastereomer, or double bond isomer thereof, wherein variables R², R³, and R⁴ are as described herein.

In certain embodiments, the present invention provides a compound of formula I,

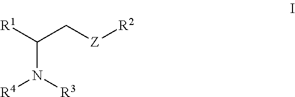

or a pharmaceutically acceptable salt, enantiomer, diastereomer, or double bond isomer thereof, wherein:

R³ is selected from:

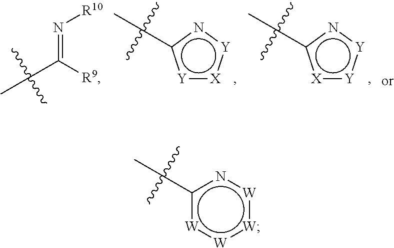

wherein:

W is independently —C(R¹²)— or N;

R¹² is halo, N(R⁵)₂, oxo, alkyl, alkenyl, alkynyl or aryl;

X is O, S, —N(R⁵)—, —C(R¹¹)—;

Y is independently —C(R¹¹)— or N;

R¹¹ is H, F, CH₃, CF₃, OH, alkyl, alkenyl, alkynyl or aryl;

R⁹ is H, alkyl, alkenyl, alkynyl, aryl, —N(R⁵)₂; and

R¹⁰ is H, alkyl, alkenyl, alkynyl, aryl, CN, —S(═O)₂—R⁶.

In certain embodiments, the present invention provides a compound of formula I,

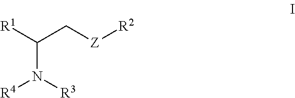

wherein:

R¹ is

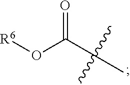

R² is a straight or branched chain aliphatic group having 10 to 25 carbon atoms and one or more double bonds;

R³ is

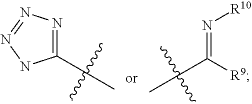

R⁴ is H;

R⁶ is H or alkyl;

R⁹ is H or alkyl;

R¹⁰ is H alkyl or CN; and

Z is S or Se.

In certain embodiments, the present invention provides a compound of formula I,

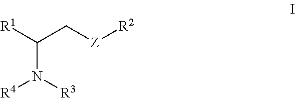

or a pharmaceutically acceptable salt, enantiomer, diastereomer, or double bond isomer thereof, wherein:

R¹ is

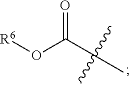

R² is a straight or branched chain aliphatic group having 10 to 25 carbon atoms and one or more double bonds;

R³ is optionally substituted

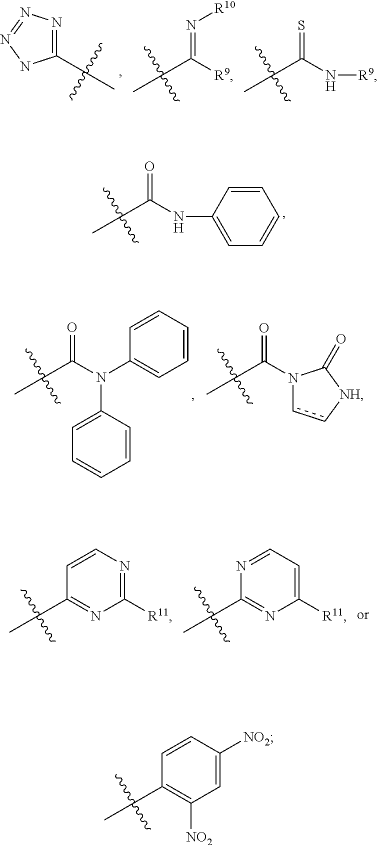

R⁴ is H;

R⁶ is H or alkyl;

R⁹ is H or alkyl;

R¹⁰ is H alkyl or CN;

R¹¹ is —NH₂, —NHNH₂ or hydrogen;

X=—N—, —C(R⁶)— or —O—;

Z is —S— or —Se—;

wherein, each of the dashed lines independently represents the presence or absence of a double bond.

In certain embodiments, the present invention provides a compound of formula Ib,

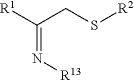

or a pharmaceutically acceptable salt, enantiomer, diastereomer, or double bond isomer thereof, wherein:

R¹ is an optionally substituted heteroaryl group or:

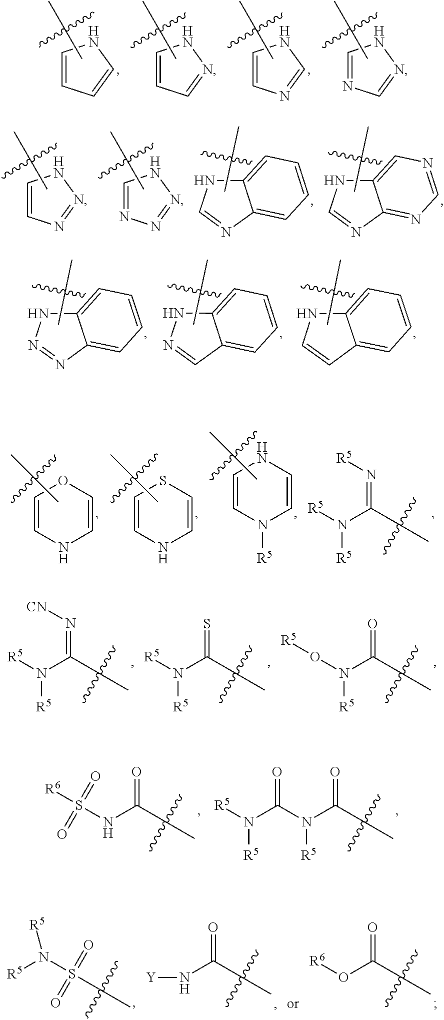

R² is an aliphatic group substituted with one or more R⁷ groups;

R¹³ is independently H,

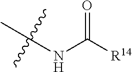

### or —NH—S(O)₂R¹⁴;

R¹⁴ is independently H, (C₁-C₄)alkyl or aryl;

R⁵ is independently H, alkyl, aryl, alkenyl, or alkynyl, wherein R⁵ is optionally substituted with one or two R⁷ groups;

R⁶ is H, alkyl, aryl, alkenyl, alkynyl, or a cyclic radical, wherein R⁶ is optionally substituted with one or two R⁷ groups;

R⁷ is —NHC(═O)(C₁-C₈)alkyl, —(C₁-C₈)alkyl, —(C₁-C₈)alkenyl, —(C₁-C₈)alkynyl, phenyl, —(C₂-C₅)heteroaryl, —(C₁-C₆)heterocycloalkyl, —(C₃-C₇)cycloalkyl, —O—(C₁-C₈)alkyl, —O—(C₁-C₈)alkenyl, —O—(C₁-C₈)alkynyl, —O-phenyl, —CN, —OH, oxo, halo, —C(═O)OH, —COhalo, —OC(═O)halo, —CF₃, N₃, NO₂, —NH₂, —NH((C₁-C₈)alkyl), —N((C₁-C₈)alkyl)₂, —NH(phenyl), —N(phenyl)₂, —C(═O)NH₂, —C(═O)NH((C₁-C₈)alkyl), —C(D)N((C₁-C₈)alkyl)₂, —C(═O)NH(phenyl), —C(═O)N(phenyl)₂, —OC(═O)NH₂, —NHOH, —NOH((C₁-C₈)alkyl), —NOH(phenyl), —OC(═O)NH((C₁-C₈)alkyl), —OC(═O)N((C₁-C₈)alkyl)₂, —OC(═O)NH(phenyl), —OC(═O)N(phenyl)₂, —CHO, —CO((C₁-C₈)alkyl), —CO(phenyl), —C(═O)O((C₁-C₈)alkyl), —C(═O)O(phenyl), —OC(═O)((C₁-C₈)alkyl), —OC(═O)(phenyl), —OC(═O)O((C₁-C₈)alkyl), —OC(═O)O(phenyl), —S—(C₁-C₈)alkyl, —S—(C₁-C₈)alkenyl, —S—(C₁-C₈)alkynyl, and —S-phenyl, —NHS(O)₂-phenyl, —NHS(O)₂-alkyl, —NHS(O)₂—(C₁-C₈)alkenyl, —NHS(O)₂—(C₁-C₈)alkynyl, —NHS(O)₂, —SC(O)-phenyl, —SC(O)-alkyl, —SC(O)—(C₁-C₈)alkenyl, —SC(O)—(C₁-C₈ alkynyl), —O—S(═O)₂—(C₁-C₈)alkyl, —O—S(═O)₂—(C₁-C₈)alkenyl, —O—S(═O)₂—(C₁-C₈)alkynyl, —O—S(═O)₂-phenyl, —(CH₂)ₙNH₂, —(CH₂)ₙ—NH((C₁-C₈)alkyl), —(CH₂)ₙN((C₁-C₈)alkyl)₂, —(CH₂)ₙNH(phenyl), or —(CH₂)ₙN(phenyl)₂, wherein n is 1 to 8.

As defined generally above, the R¹ group of formulae I is a heteroaryl group or: is selected from a heteroaryl group or:

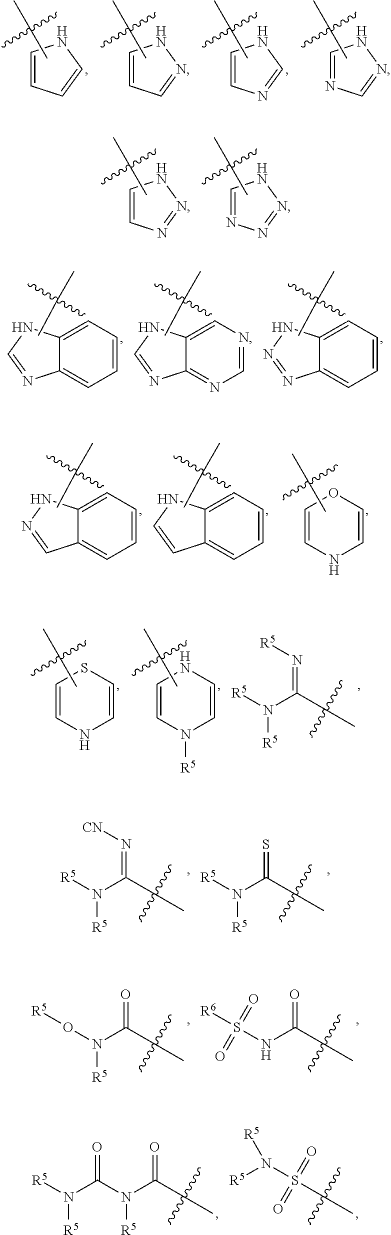

In certain embodiments, R¹ is

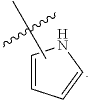

In certain embodiments, R¹ is

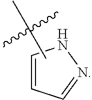

In certain embodiments, R¹ is

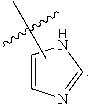

In certain embodiments, R¹ is

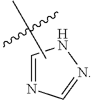

In certain embodiments, R¹ is

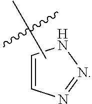

In certain embodiments, R¹ is

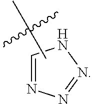

In certain embodiments, R¹ is

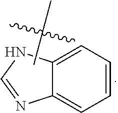

In certain embodiments, R¹ is

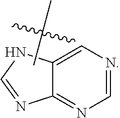

In certain embodiments, R¹ is

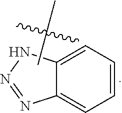

In certain embodiments, R¹ is

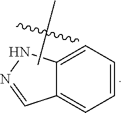

In certain embodiments, R¹ is

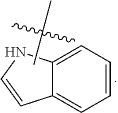

In certain embodiments, R¹ is

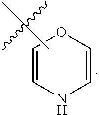

In certain embodiments, R¹ is

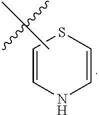

In certain embodiments, R¹ is

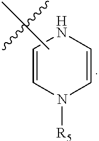

In certain embodiments, R¹ is

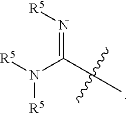

In certain embodiments, R¹ is

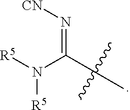

In certain embodiments, R¹ is

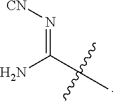

In certain embodiments, R¹ is

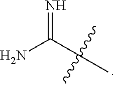

In certain embodiments, R¹ is

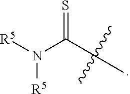

In certain embodiments, R¹ is

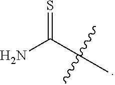

In certain embodiments, R¹ is

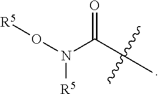

In certain embodiments, R¹ is

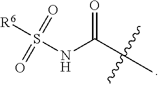

In certain embodiments, R¹ is

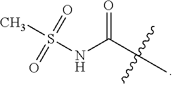

In certain embodiments, R¹ is

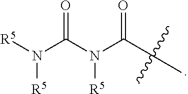

In certain embodiments, R¹ is

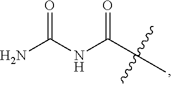

when each R⁵ is hydrogen. In certain embodiments, R¹ is

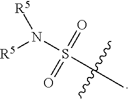

In certain embodiments, R¹ is

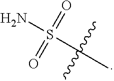

In certain embodiments, R¹ is

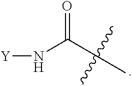

In certain embodiments, R¹ is

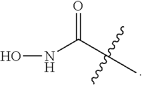

In certain embodiments, R¹ is

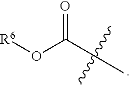

In certain embodiments, R⁶ is hydrogen. In certain embodiments, R⁶ is methyl. In certain embodiments, R⁶ is ethyl. In certain embodiments, R⁶ is propyl. In certain embodiments, R⁶ is butyl. In certain embodiments, R⁶ is pentyl. In certain embodiments, R⁶ is hexyl.

As defined generally above, the R² group of formulae I or Ia is an aliphatic group substituted with one or more R⁷ groups. In certain embodiments, R² is an aliphatic group substituted with one R⁷ group. In certain embodiments, R² is an aliphatic group substituted with two R⁷ groups. In certain embodiments, R² is selected from:

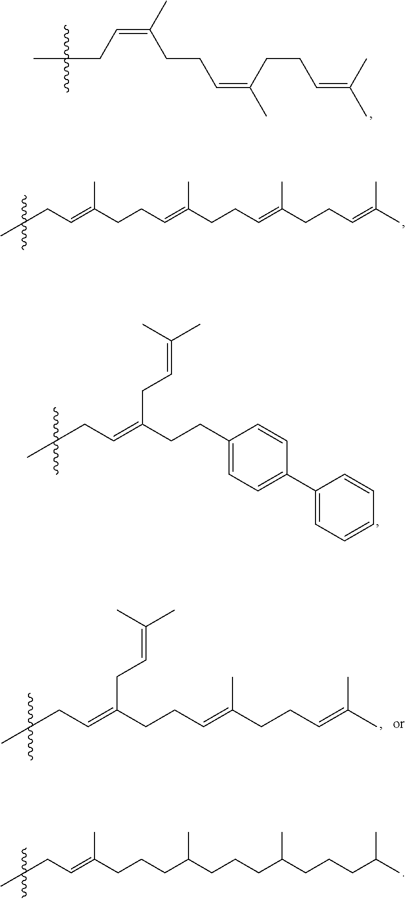

in any isomeric form, optionally substituted with one or more R⁷ groups. In certain embodiments, R² is

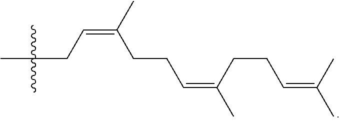

In certain embodiments, R² is

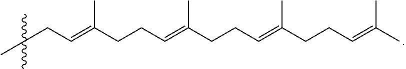

In certain embodiments, R² is

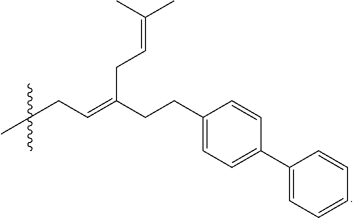

In certain embodiments, R² is

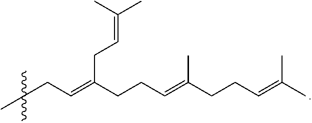

In certain embodiments, R² is

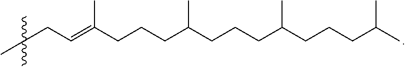

In certain embodiments, R² is a straight chain or branched aliphatic group, substituted or unsubstituted, having 10-25 carbon atoms, optionally having one or more double bonds. In certain embodiments, R² is a straight or branched chain alkyl group, having 10 to 25 carbon atoms or a straight or branched chain alkenyl group, having 10 to 25 carbon atoms and one or more double bonds.

As defined generally above, the R³ group of formulae I or Ia is a heteroaryl group,

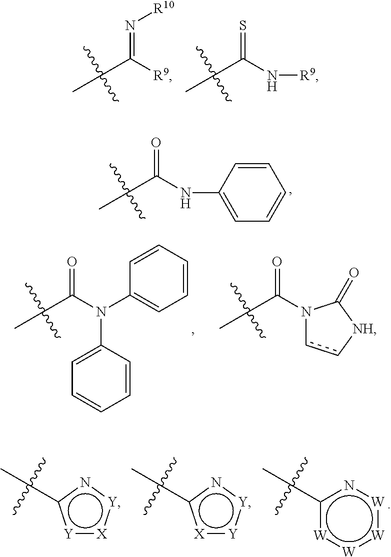

or

In certain embodiments, R³ is a heteroaryl group. In certain embodiments R³ is

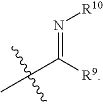

In certain embodiments R³ is

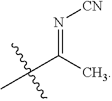

In certain embodiments R³ is

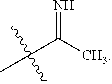

In certain embodiments R³ is

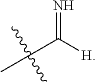

In certain embodiments R³ is

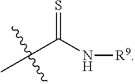

In certain embodiments R³ is

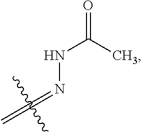

when R⁴ is not present. In certain embodiments R³ is

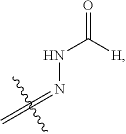

when R⁴ is not present. In certain embodiments R³ is

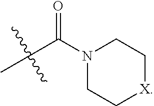

In certain embodiments R³ is

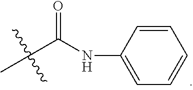

In certain embodiments R³ is

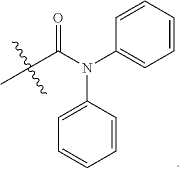

In certain embodiments R³ is

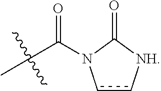

In certain embodiments, R³ is

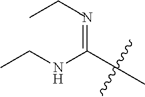

when R⁹ is —NHR⁶, R⁶ is hydrogen and R¹⁰ is —CH₂CH₃. In certain embodiments, R³ is

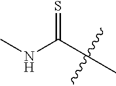

when R⁹ is —CH₃. In certain embodiments, R³ is

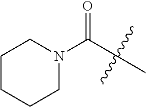

when X is —CH₂—. In certain embodiments, R³ is

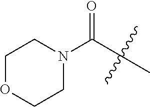

when X is O. In certain embodiments, R³ is

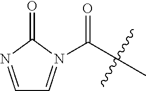

when each of the dashed lines is a double bond. In certain embodiments, R³ is

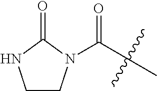

when each of the dashed lines is a single bond. In certain embodiments, R³ is

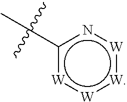

In certain embodiments, R³ is

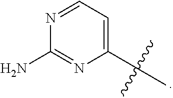

In certain embodiments, R³ is

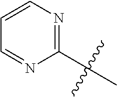

when R¹¹ is hydrogen. In certain embodiments, R³ is

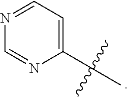

In certain embodiments, R³

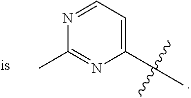

In certain embodiments, R³ is

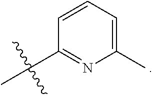

In certain embodiments, R³ is

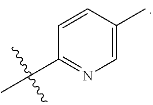

In certain embodiments, R³ is

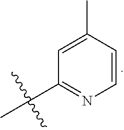

In certain embodiments, R³ is

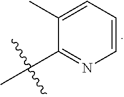

In certain embodiments, R³ is

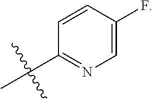

In certain embodiments, R³ is

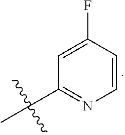

In certain embodiments, R³ is

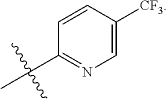

In certain embodiments, R³ is

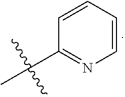

In certain embodiments, R³ is

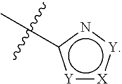

In certain embodiments, R³ is

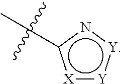

In certain embodiments, R³ is

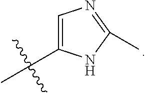

In certain embodiments, R³ is

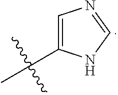

In certain embodiments, R³ is

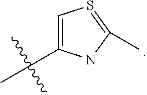

In certain embodiments, R³ is

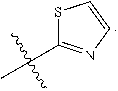

In certain embodiments, R³ is

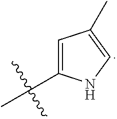

In certain embodiments, R³ is

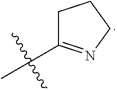

In certain embodiments, R³ is

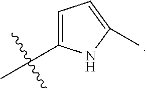

In certain embodiments, R³ is

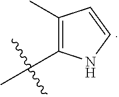

In certain embodiments, R³ is

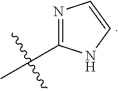

In certain embodiments, R³ is

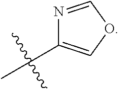

In certain embodiments, R³ is

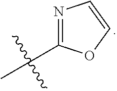

As defined generally above, the R⁴ group of formulae I or Ia is H, alkyl, aryl, alkenyl, alkynyl, or a cyclic radical, wherein R⁴ is optionally substituted with one or two R⁷ groups. In certain embodiments, R⁴ is hydrogen. In certain embodiments, R⁴ is methyl. In certain embodiments, R⁴ is ethyl. In certain embodiments, R⁴ is propyl. In certain embodiments, R⁴ is butyl. In certain embodiments, R⁴ is pentyl. In certain embodiments, R⁴ is hexyl.

As defined generally above, the R⁵ group of formulae I or Ia is independently H, alkyl, aryl, alkenyl, or alkynyl, wherein R⁵ is optionally substituted with one or two R⁷ groups. In certain embodiments, R⁵ is hydrogen. In certain embodiments, R⁵ is methyl. In certain embodiments, R⁵ is ethyl. In certain embodiments, R⁵ is propyl. In certain embodiments, R⁵ is butyl. In certain embodiments, R⁵ is pentyl. In certain embodiments, R⁵ is hexyl.

As defined generally above, the R⁶ group of formulae I or Ia is H, alkyl, aryl, alkenyl, alkynyl, or a cyclic radical, wherein R⁶ is optionally substituted with one or two R⁷ groups. In certain embodiments, R⁶ is hydrogen. In certain embodiments, R⁶ is methyl. In certain embodiments, R⁶ is ethyl. In certain embodiments, R⁶ is propyl. In certain embodiments, R⁶ is butyl. In certain embodiments, R⁶ is pentyl. In certain embodiments, R⁶ is hexyl.

As defined generally above, the R⁷ group of formulae I or Ia is —NHC(═O)(C₁-C₈)alkyl, —(C₁-C₈)alkyl, —(C₁-C₈)alkenyl, —(C₁-C₈)alkynyl, phenyl, —(C₂-C₅)heteroaryl, —(C₁-C₆)heterocycloalkyl, —(C₃-C₇)cycloalkyl, —O—(C₁-C₈)alkyl, —O—(C₁-C₈)alkenyl, —O—(C₁-C₈)alkynyl, —O-phenyl, —CN, —OH, oxo, halo, —C(═O)OH, —COhalo, —OC(═O)halo, —CF₃, N₃, NO₂, —NH₂, —NH((C₁-C₈)alkyl), —N((C₁-C₈)alkyl)₂, —NH(phenyl), —N(phenyl)₂, —C(═O)NH₂, —C(═O)NH((C₁-C₈)alkyl), —C(═O)N((C₁-C₈)alkyl)₂, —C(═O)NH(phenyl), —C(═O)N(phenyl)₂, —OC(═O)NH₂, —NHOH, —NOH((C₁-C₈)alkyl), —NOH(phenyl), —OC(═O)NIA(C₁-C₈)alkyl), —OC(═O)N((C₁-C₈)alkyl)₂, —OC(═O)NH(phenyl), —OC(═O)N(phenyl)₂, —CHO, —CO((C₁-C₈)alkyl), —CO(phenyl), —C(═O)O((C₁-C₈)alkyl), —C(═O)O(phenyl), —OC(═O)((C₁-C₈)alkyl), —OC(═O)(phenyl), —OC(═O)O((C₁-C₈)alkyl), —OC(═O)O(phenyl), —S—(C₁-C₈)alkyl, —S—(C₁-C₈)alkenyl, —S—(C₁-C₈)alkynyl, and —S-phenyl, —NHS(O)₂-phenyl, —NHS(O)₂-alkyl, —NHS(O)₂—(C₁-C₈)alkenyl, —NHS(O)₂—(C₁-C₈)alkynyl, —NHS(O)₂, —SC(O)-phenyl, —SC(O)-alkyl, —SC(O)—(C₁-C₈)alkenyl, —SC(O)—(C₁-C₈ alkynyl), —O—S(═O)₂—(C₁-C₈)alkyl, —O—S(═O)₂—(C₁-C₈)alkenyl, —O—S(═O)₂—(C₁-C₈)alkynyl, —O—S(═O)₂-phenyl, —(CH₂)ₙNH₂, —(CH₂)ₙ—NH((C₁-C₈)alkyl), —(CH₂)ₙN((C₁-C₈)alkyl)₂, —(CH₂)ₙNH(phenyl), or —(CH₂)ₙN(phenyl)₂, wherein n is 1 to 8. In certain embodiments, R⁷ is any single one of the moieties listed within the general definition of R⁷.

As defined generally above, the R⁹ group of formulae I or Ia is H, alkyl, alkenyl, alkynyl, aryl, —N(R⁵)₂. In certain embodiments, R⁹ is hydrogen. In certain embodiments, R⁹ is methyl. In certain embodiments, R⁹ is ethyl. In certain embodiments, R⁹ is propyl. In certain embodiments, R⁹ is butyl. In certain embodiments, R⁹ is pentyl. In certain embodiments, R⁹ is hexyl. In certain embodiments, R⁹ is an alkyl group, optionally substituted with one or two R⁷ groups. In certain embodiments, R⁹ is methyl.

As defined generally above, the R¹⁰ group of formulae I or Ia is H, alkyl, alkenyl, alkynyl, aryl, CN, —S(═O)₂—R⁶. In certain embodiments, R¹⁰ is hydrogen. In certain embodiments, R¹⁰ is methyl. In certain embodiments, R¹⁰ is ethyl. In certain embodiments, R¹⁰ is propyl. In certain embodiments, R¹⁰ is butyl. In certain embodiments, R¹⁰ is pentyl. In certain embodiments, R¹⁰ is hexyl. In certain embodiments, R¹⁰ is —CN.

As defined generally above, the R¹¹ group of formulae I or Ia is —NH₂, —NHNH₂ or hydrogen. In certain embodiments, R¹¹ is —NH₂. In certain embodiments, R¹¹ is —NHNH₂. In certain embodiments, R¹¹ is hydrogen.

As defined generally above, the X group of formulae I or Ia is —N—, —C(R⁶)— or —O—. In certain embodiments, X is —N—. In certain embodiments, X is —C(R⁶)—. In certain embodiments, X is —O—.

As defined generally above, the Z group of formulae I or Ia is —S—, —O—, —Se—, —S(O)—, —SO₂—, or —NH—. In certain embodiments, Z is —S—. In certain embodiments, Z is —O—. In certain embodiments, Z is —Se—. In certain embodiments, Z is —S(O)—. In certain embodiments, Z is —SO₂—. In certain embodiments, Z is —NH—.

As defined generally herein, each of the dashed lines of formulae I, Ia and/or Ib independently represents the presence or absence of a double bond. In certain embodiments, each of the dashed lines independently represents the presence of a double bond. In certain embodiments, each of the dashed lines independently represents the presence of a single bond.

As defined generally above, the R¹ group of formula Ib is an optionally substituted heteroaryl group or:

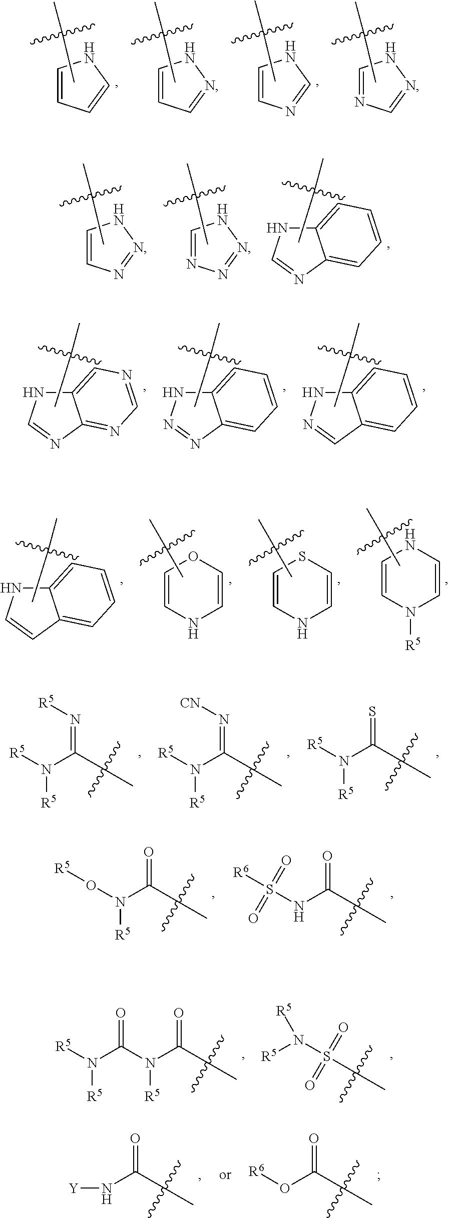

As defined generally above, the R¹ group of formula Ib is R² is an aliphatic group substituted with one or more R⁷ groups.

As defined generally above, the R¹³ group of formula Ib is independently H,

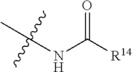

### or —NH—S(O)₂R¹⁴.

As defined generally above, the R¹⁴ group of formula Ib is independently H, (C₁-C₄)alkyl or aryl.

As defined generally above, the R⁵ group of formula Ib is independently H, alkyl, aryl, alkenyl, or alkynyl, wherein R⁵ is optionally substituted with one or two R⁷ groups.

As defined generally above, the R⁶ group of formula Ib is H, alkyl, aryl, alkenyl, alkynyl, or a cyclic radical, wherein R⁶ is optionally substituted with one or two R⁷ groups.

As defined generally above, the R⁷ group of formula Ib is —NHC(═O)(C₁-C₈)alkyl, —(C₁-C₈)alkyl, —(C₁-C₈)alkenyl, —(C₁-C₈)alkynyl, phenyl, —(C₂-C₅)heteroaryl, —(C₁-C₆)heterocycloalkyl, —(C₃-C₇)cycloalkyl, —O—(C₁-C₈)alkyl, —O—(C₁-C₈)alkenyl, —O—(C₁-C₈)alkynyl, —O-phenyl, —CN, —OH, oxo, halo, —C(═O)OH, —COhalo, —OC(═O)halo, —CF₃, N₃, NO₂, —NH₂, —NH((C₁-C₈)alkyl), —N((C₁-C₈)alkyl)₂, —NH(phenyl), —N(phenyl)₂, —C(═O)NH₂, —C(═O)NH((C₁-C₈)alkyl), —C(═O)N((C₁-C₈)alkyl)₂, —C(═O)NH(phenyl), —C(═O)N(phenyl)₂, —OC(═O)NH₂, —NHOH, —NOH((C₁-C₈)alkyl), —NOH(phenyl), —OC(═O)NH((C₁-C₈)alkyl), —OC(═O)N((C₁-C₈)alkyl)₂, —OC(═O)NH(phenyl), —OC(═O)N(phenyl)₂, —CHO, —CO((C₁-C₈)alkyl), —CO(phenyl), —C(═O)O((C₁-C₈)alkyl), —C(═O)O(phenyl), —OC(═O)((C₁-C₈)alkyl), —OC(═O)(phenyl), —OC(═O)O((C₁-C₈)alkyl), —OC(═O)O(phenyl), —S—(C₁-C₈)alkyl, —S—(C₁-C₈)alkenyl, —S—(C₁-C₈)alkynyl, and —S-phenyl, —NHS(O)₂-phenyl, —NHS(O)₂-alkyl, —NHS(O)₂—(C₁-C₈)alkenyl, —NHS(O)₂—(C₁-C₈)alkynyl, —NHS(O)₂, —SC(O)-phenyl, —SC(O)-alkyl, —SC(O)—(C₁-C₈)alkenyl, —SC(O)—(C₁-C₈ alkynyl), —O—S(═O)₂—(C₁-C₈)alkyl, —O—S(═O)₂—(C₁-C₈)alkenyl, —O—S(═O)₂—(C₁-C₈)alkynyl, —O—S(═O)₂-phenyl, —(CH₂)ₙNH₂, —(CH₂)ₙ—NH((C₁-C₈)alkyl), —(CH₂)N(C₁-C₈)alkyl)₂, —(CH₂)ₙNH(phenyl), or —(CH₂)ₙN(phenyl)₂, wherein n is 1 to 8.

Exemplary compounds of the present invention are set forth in Table 1 below.

TABLE 1
Exemplary Compounds 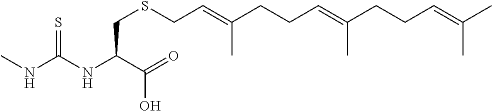
A 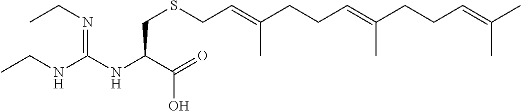
B 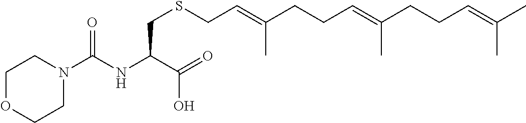
C 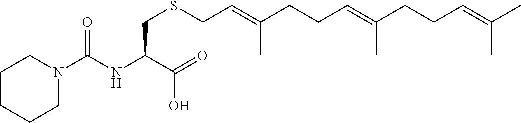
D 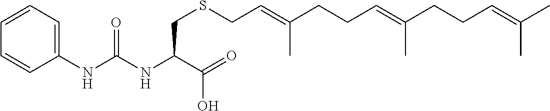
E 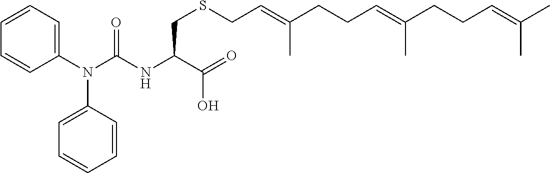
F 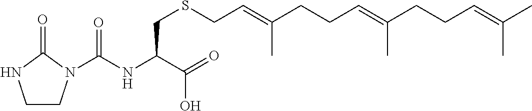
G 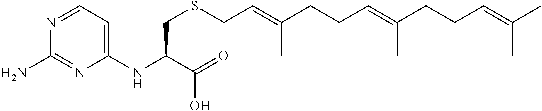
H 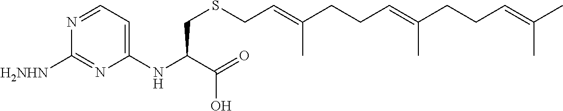
I 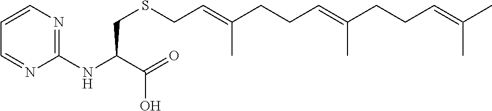
J 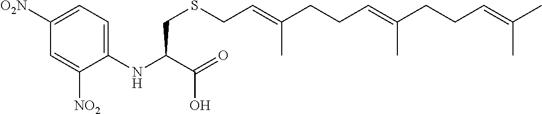
K.

In certain embodiments, the present invention provides any compound depicted in Table 1, above, or a pharmaceutically acceptable salt thereof.

Additional exemplary compounds of the present invention are set forth in Table 2 below.

TABLE 2
Exemplary Compounds

| Com- | | | |
|---|---|---|---|
| pound | | | |
| Num- | | Mol. | IUPAC |
| ber | Structure | Weight | Name |
| L-1 | 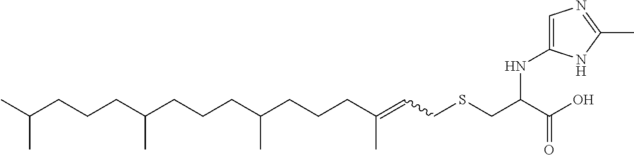 | 479.76 | 2-[(2-methyl- 1H-imidazol-5- yl)amino]-3- [(3,7,11,15- tetramethyl- hexadec-2-en-1- yl)sulfanyl] propanoic acid |
| L-2 | 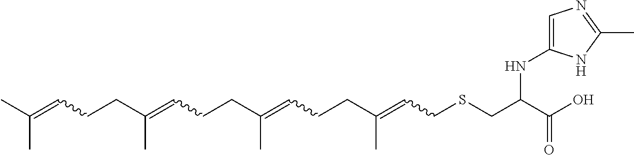 | 473.71 | 2-[(2-methyl- 1H-imidazol-5- yl)amino]-3- {[3,7,11,15- tetramethyl- hexadeca- 2,6,10,14- tetraen-1-yl] sulfanyl} propanoic acid |
| L-3 | 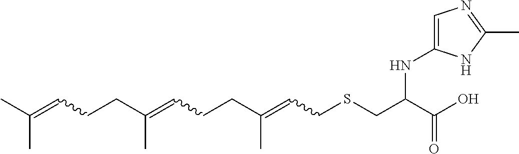 | 405.60 | 2-[(2-methyl- 1H-imidazol-5- yl)amino]-3- [(3,7,11- trimethyldodeca- 2,6,10-trien-1- yl)sulfanyl] propanoic acid |
| L-4 | 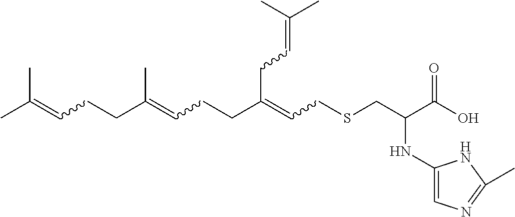 | 459.69 | 3-{[7,11-dimethyl- 3-(3-methylbut-2- en-1-yl)dodeca- 2,6,10-trien-1- yl]sulfanyl}-2- [(2-methyl-1H- imidazol-5-yl) amino] propanoic acid |
| L-5 | 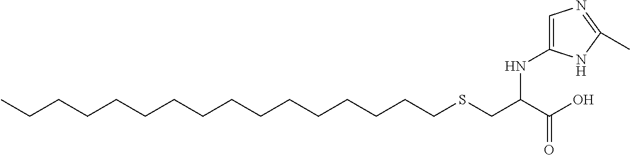 | 425.67 | 3- (hexadecylsulfanyl)- 2-[(2-methyl- 1H-imidazol- 5-yl)amino] propanoic acid |
| L-6 | 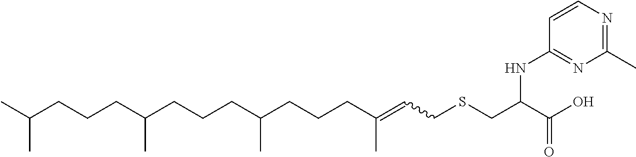 | 491.77 | 2-[(2- methylpyrimidin- 4-yl)amino]- 3-[(3,7,11,15- tetramethylhexadec- 2- en-1-yl)sulfanyl] propanoic acid |
| L-7 | 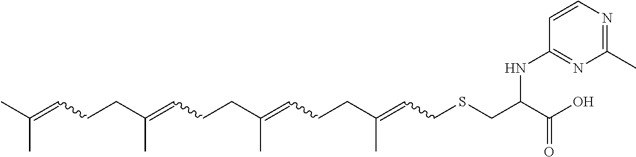 | 485.72 | 2-[(2- methylpyrimidin- 4-yl)amino]- 3-{[3,7,11,15- tetramethylhexadeca- 2,6,10,14-tetraen-1- yl]sulfanyl} propanoic acid |
| L-8 | 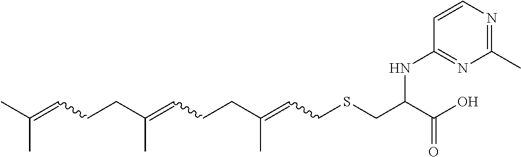 | 417.61 | 2-[(2- methylpyrimidin- 4-yl)amino]- 3-[(3,7,11- trimethyldodeca- 2,6,10- trien-1-yl) sulfanyl] propanoic acid |
| L-9 | 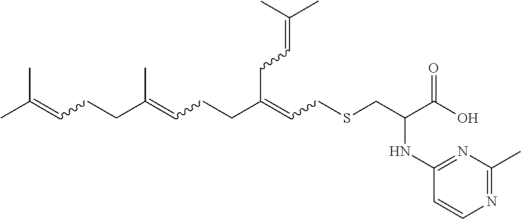 | 471.70 | 3-{[7,11- dimethyl-3-(3- methylbut-2- en-1-yl)dodeca- 2,6,10-trien-1- yl]sulfanyl}-2- [(2-methylpyrimidin- 4-yl)amino] propanoic acid |
| L-10 | 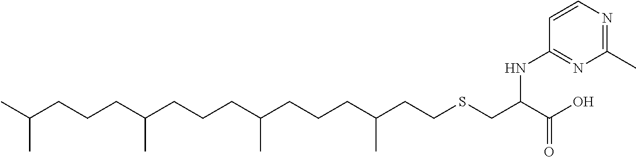 | 437.68 | 3- (hexadecylsulfanyl)- 2-[(2- methylpyrimidin-4- yl)amino] propanoic acid |
| L-11 | 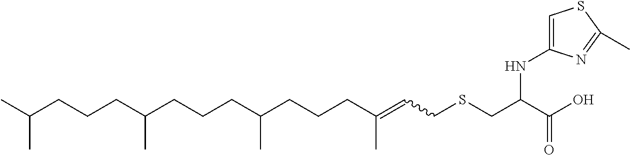 | 496.81 | 2-[(2-methyl- 1,3-thiazol-4- yl)amino]-3- [(3,7,11,15- tetramethylhexadec- 2-en-1- yl)sulfanyl] propanoic acid |
| L-12 | 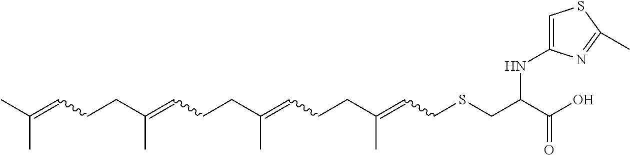 | 490.76 | 2-[(2-methyl- 1,3-thiazol-4- yl)amino]-3- {[3,7,11,15- tetramethylhexadeca- 2,6,10,14- tetraen-1-yl] sulfanyl} propanoic acid |
| L-13 | 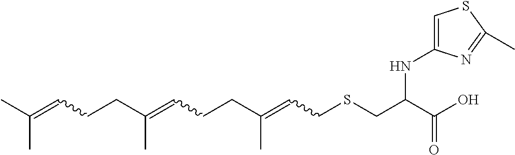 | 422.65 | 2-[(2-methyl- 1,3-thiazol-4- yl)amino]-3- [(3,7,11- trimethyldodeca- 2,6,10-trien-1- yl)sulfanyl] propanoic acid |
| L-14 | 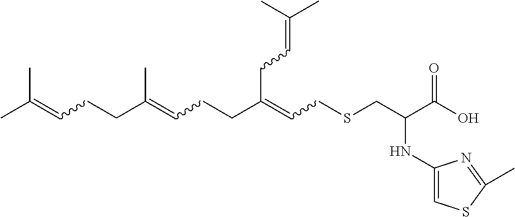 | 476.74 | 3-{[7,11-dimethyl- 3-(3-methylbut-2- en-1-yl)dodeca- 2,6,10-trien-1- yl]sulfanyl}-2- [(2-methyl-1,3- thiazol-4-yl) amino] propanoic acid |
| L-15 | 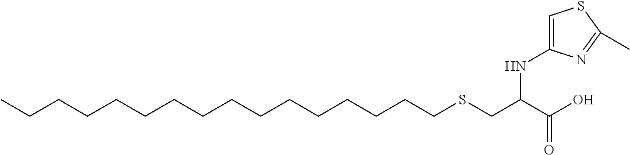 | 442.72 | 3- (hexadecylsulfanyl)- 2-[(2-methyl- 1,3-thiazol-4-yl) amino]propanoic acid |
| L-16 | 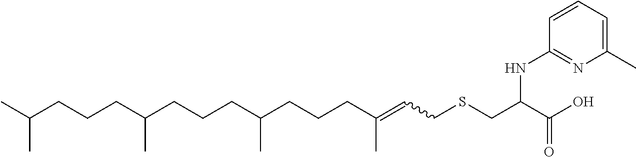 | 490.79 | 2-[(6- methylpyridin- 2-yl)amino]-3- [(3,7,11,15- tetramethylhexadec- 2- en-1-yl)sulfanyl] propanoic acid |
| L-17 | 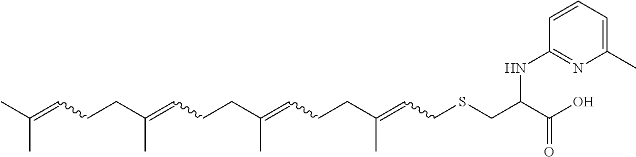 | 484.74 | 2-[(6- methylpyridin- 2-yl)amino]-3- {[3,7,11,15- tetramethylhexadeca- 2,6,10,14-tetraen-1- yl]sulfanyl} propanoic acid |
| L-18 | 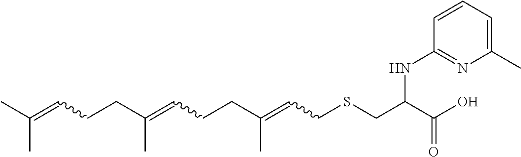 | 416.62 | 2-[(6- methylpyridin- 2-yl)amino]-3- [(3,7,11- trimethyldodeca- 2,6,10- trien-1-yl) sulfanyl] propanoic acid |
| L-19 | 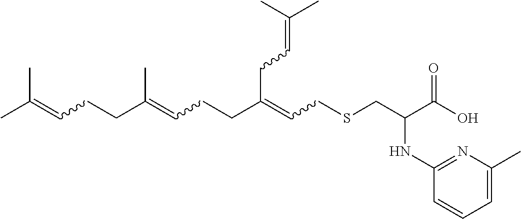 | 470.71 | 3-{[7,11-dimethyl- 3-(3-methylbut-2- en-1-yl)dodeca- 2,6,10-trien-1- yl]sulfanyl}-2- [(6-methylpyridin-2- yl)amino] propanoic acid |
| L-20 | 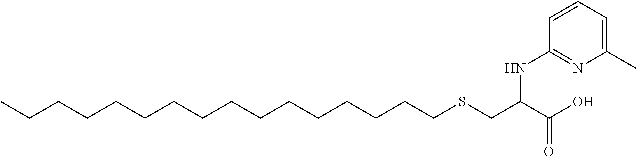 | 436.69 | 3- (hexadecylsulfanyl)- 2-[(6- methylpyridin-2- yl)amino]propanoic acid |
| L-21 | 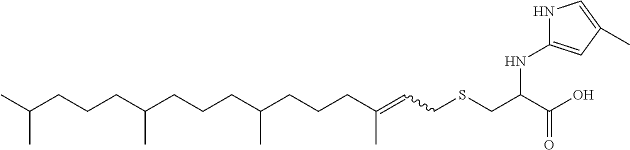 | 478.77 | 2-[(4-methyl-1H- pyrrol-2-yl)amino]- 3-[(3,7,11,15- tetramethylhexadec- 2- en-1-yl)sulfanyl] propanoic acid |
| L-22 | 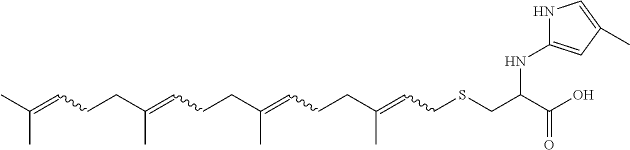 | 472.73 | 2-[(4-methyl-1H- pyrrol-2-yl)amino]- 3-{[3,7,11,15- tetramethylhexadeca- 2,6,10,14-tetraen-1- yl]sulfanyl} propanoic acid |
| L-23 | 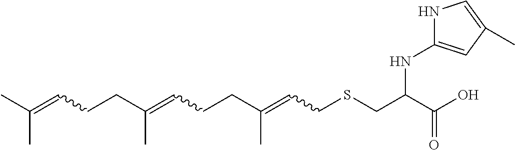 | 404.61 | 2-[(4-methyl-1H- pyrrol-2-yl)amino]- 3-[(3,7,11- trimethyldodeca- 2,6,10- trien-1-yl)sulfanyl] propanoic acid |
| L-24 | 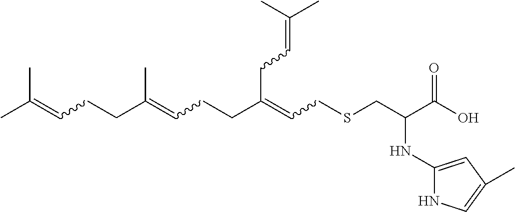 | 458.70 | 3-{[7,11-dimethyl- 3-(3-methylbut-2- en-1-yl)dodeca- 2,6,10-trien-1- yl]sulfanyl}-2- [(4-methyl- 1H-pyrrol- 2-yl)amino] propanoic acid |
| L-25 | 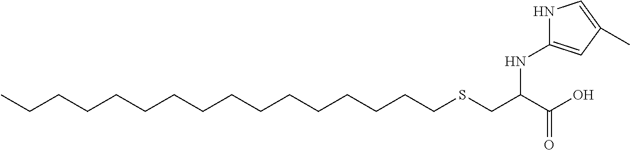 | 424.68 | 3- (hexadecylsulfanyl)- 2-[(4-methyl- 1H-pyrrol-2-yl) amino]propanoic acid |
| L-26 | 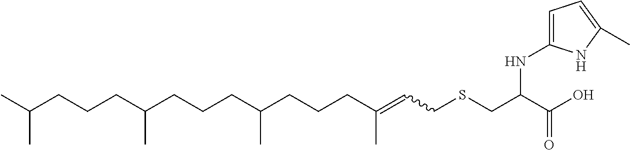 | 478.77 | 2-[(5-methyl-1H- pyrrol-2-yl)amino]- 3-[(3,7,11,15- tetramethylhexadec- 2- en-1-yl)sulfanyl] propanoic acid |
| L-27 | 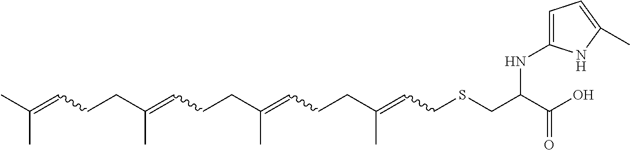 | 472.73 | 2-[(5-methyl-1H- pyrrol-2-yl)amino]- 3-{[3,7,11,15- tetramethylhexadeca- 2,6,10,14-tetraen-1- yl]sulfanyl} propanoic acid |
| L-28 | 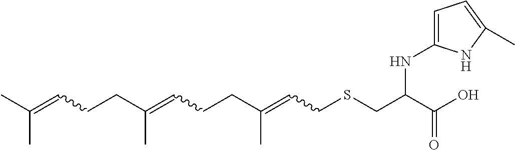 | 404.61 | 2-[(5-methyl-1H- pyrrol-2-yl)amino]- 3-[(3,7,11- trimethyldodeca- 2,6,10- trien-1-yl)sulfanyl] propanoic acid |
| L-29 | 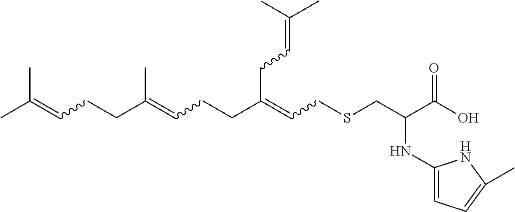 | 458.70 | 3-{[7,11-dimethyl- 3-(3-methylbut-2- en-1-yl)dodeca- 2,6,10-trien-1- yl]sulfanyl}-2-[(5- methyl-1H-pyrrol- 2-yl)amino] propanoic acid |
| L-30 | 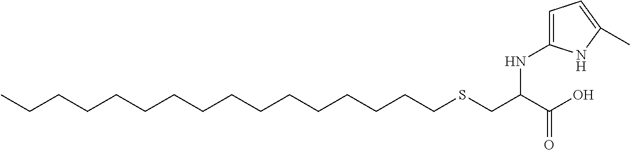 | 424.68 | 3- (hexadecylsulfanyl)- 2-[(5-methyl- 1H-pyrrol-2-yl) amino]propanoic acid |
| L-31 | 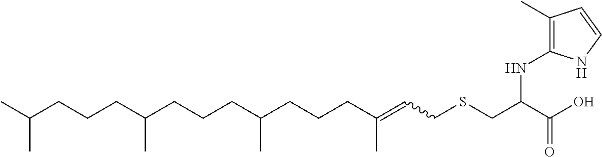 | 478.77 | 2-[(3-methyl-1H- pyrrol-2-yl)amino]- 3-[(3,7,11,15- tetramethylhexadec- 2- en-1-yl)sulfanyl] propanoic acid |
| L-32 | 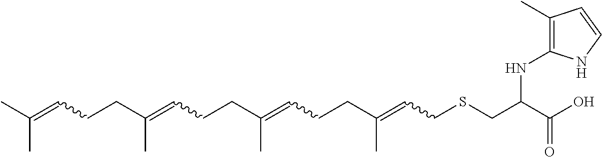 | 472.73 | 2-[(3-methyl-1H- pyrrol-2-yl)amino]- 3-{[3,7,11,15- tetramethylhexadeca- 2,6,10,14-tetraen-1- yl]sulfanyl} propanoic acid |
| L-33 | 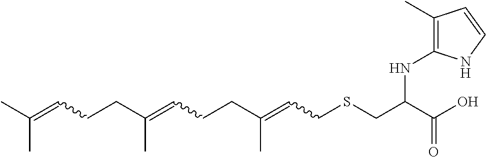 | 404.61 | 2-[(3-methyl-1H- pyrrol-2-yl)amino]- 3-[(3,7,11- trimethyldodeca-2,6,10- trien-1-yl)sulfanyl] propanoic acid |
| L-34 | 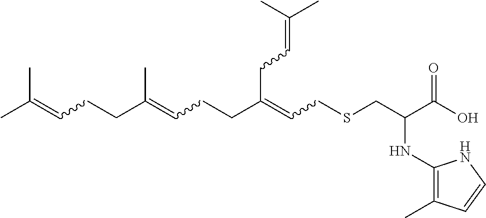 | 458.70 | 3-{[7,11-dimethyl- 3-(3-methylbut-2- en-1-yl)dodeca- 2,6,10-trien-1- yl]sulfanyl}-2-[(3- methyl-1H-pyrrol- 2-yl)amino] propanoic acid |
| L-35 | 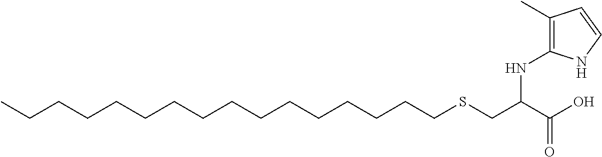 | 424.68 | 3- (hexadecylsulfanyl)- 2-[(3-methyl- 1H-pyrrol-2-yl) amino]propanoic acid |
| L-36 | 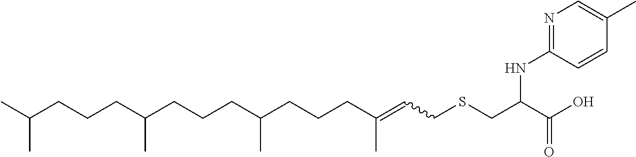 | 490.78 | 2-[(5- methylpyridin-2- yl)amino]-3- [(3,7,11,15- tetramethylhexadec- 2- en-1-yl)sulfanyl] propanoic acid |
| L-37 | 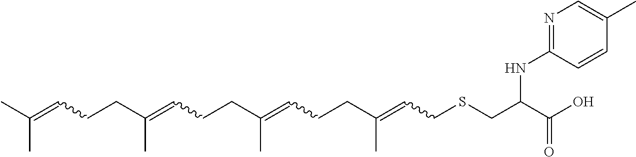 | 484.74 | 2-[(5- methylpyridin-2- yl)amino]-3- {[3,7,11,15- tetramethylhexadeca- 2,6,10,14-tetraen-1- yl]sulfanyl} propanoic acid |
| L-38 | 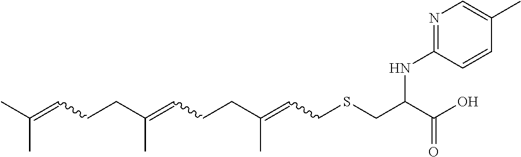 | 416.62 | 2-[(5- methylpyridin-2- yl)amino]-3- [(3,7,11- trimethyldodeca- 2,6,10- trien-1-yl) sulfanyl] propanoic acid |
| L-39 | 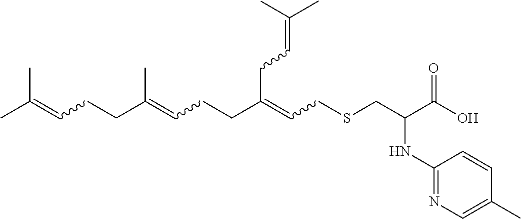 | 470.71 | 3-{[7,11- dimethyl-3-(3- methylbut-2- en-1-yl)dodeca- 2,6,10-trien-1- yl]sulfanyl}-2- [(5- methylpyridin-2- yl)amino] propanoic acid |
| L-40 | 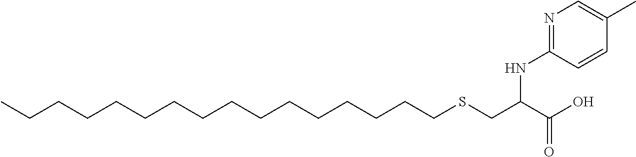 | 436.69 | 3- (hexadecylsulfanyl)- 2-[(5- methylpyridin-2- yl)amino]propanoic acid |
| L-41 | 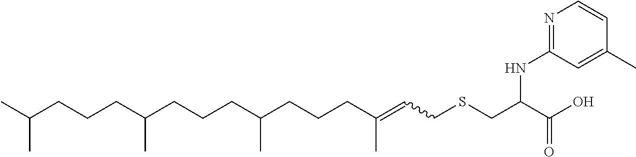 | 490.78 | 2-[(4- methylpyridin-2- yl)amino]-3- [(3,7,11,15- tetramethylhexadec- 2- en-1-yl)sulfanyl] propanoic acid |
| L-42 | 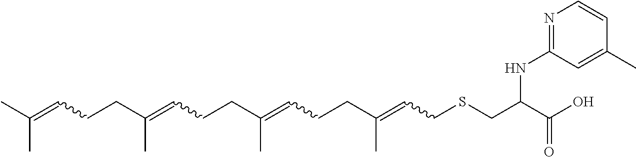 | 484.74 | 2-[(4- methylpyridin-2- yl)amino]-3- {[3,7,11,15- tetramethylhexadeca- 2,6,10,14-tetraen-1- yl]sulfanyl} propanoic acid |
| L-43 | 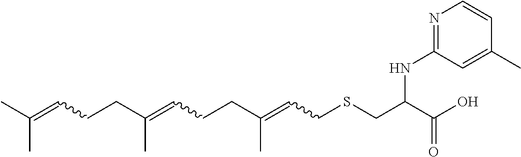 | 416.62 | 2-[(4- methylpyridin-2- yl)amino]-3- [(3,7,11- trimethyldodeca- 2,6,10- trien-1-yl)sulfanyl] propanoic acid |
| L-44 | 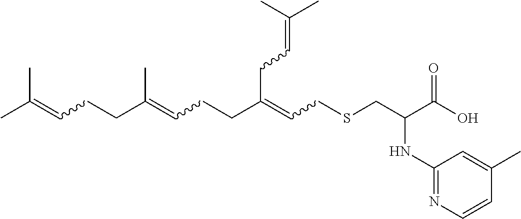 | 470.71 | 3-{[7,11-dimethyl- 3-(3-methylbut-2- en-1-yl)dodeca- 2,6,10-trien-1- yl]sulfanyl}-2- [(4-methylpyridin-2- yl)amino] propanoic acid |
| L-45 | 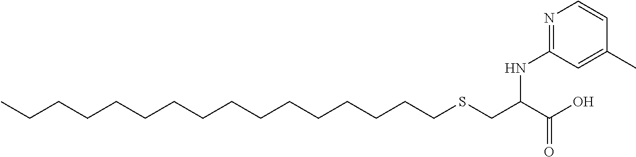 | 436.69 | 3- (hexadecylsulfanyl)- 2-[(4- methylpyridin-2- yl)amino]propanoic acid |
| L-46 | 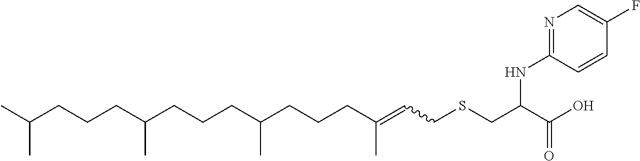 | 494.75 | 2-[(5- fluoropyridin-2-yl) amino]-3- [(3,7,11,15- tetramethylhexadec- 2- en-1-yl)sulfanyl] propanoic acid |
| L-47 | 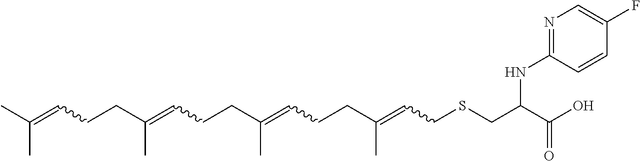 | 488.70 | 2-[(5- fluoropyridin-2- yl)amino]-3- {[3,7,11,15- tetramethylhexadeca- 2,6,10,14-tetraen-1- yl]sulfanyl} propanoic acid |
| L-48 | 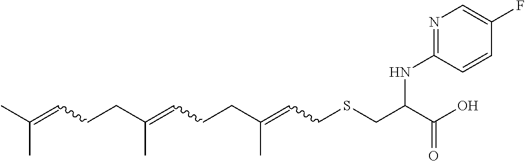 | 420.58 | 2-[(5- fluoropyridin-2- yl)amino]-3- [(3,7,11- trimethyldodeca- 2,6,10- trien-1-yl)sulfanyl] propanoic acid |
| L-49 | 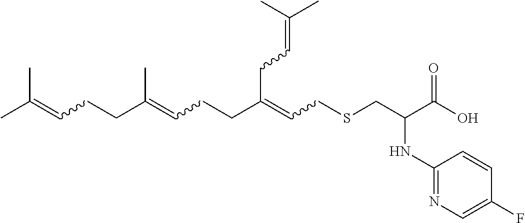 | 474.67 | 3-{[7,11-dimethyl- 3-(3-methylbut-2- en-1-yl)dodeca- 2,6,10-trien-1- yl]sulfanyl}-2- [(5-fluoropyridin-2- yl)amino] propanoic acid |
| L-50 | 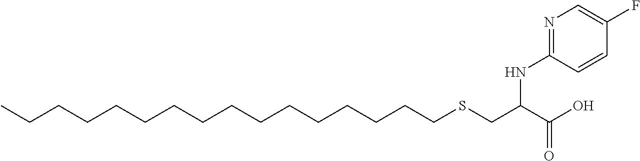 | 440.66 | 2-[(5- fluoropyridin-2- yl)amino]-3- (hexadecylsulfanyl) propanoic acid |
| L-51 | 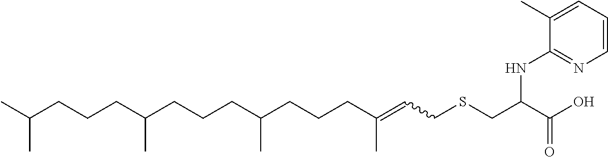 | 490.79 | 2-[(3- methylpyridin-2- yl)amino]-3- [(3,7,11,15- tetramethylhexadec- 2- en-1-yl)sulfanyl] propanoic acid |
| L-52 | 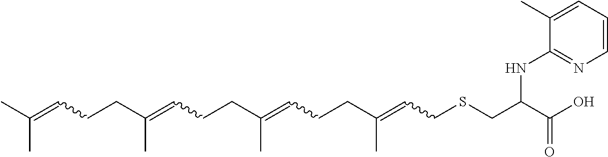 | 484.74 | 2-[(3- methylpyridin-2- yl)amino]-3- {[3,7,11,15- tetramethylhexadeca- 2,6,10,14-tetraen-1- yl]sulfanyl} propanoic acid |
| L-53 | 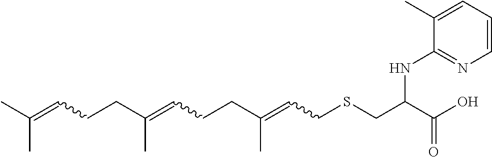 | 416.62 | 2-[(3- methylpyridin-2- yl)amino]-3- [(3,7,11- trimethyldodeca- 2,6,10- trien-1-yl) sulfanyl] propanoic acid |
| L-54 | 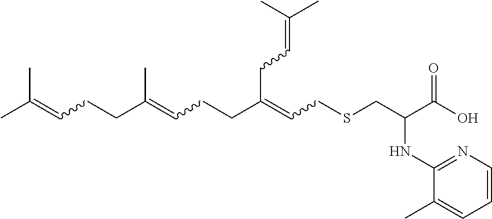 | 470.71 | 3-{[7,11-dimethyl- 3-(3-methylbut-2- en-1-yl)dodeca- 2,6,10-trien-1- yl]sulfanyl}-2- [(3-methylpyridin- 2- yl)amino] propanoic acid |
| L-55 | 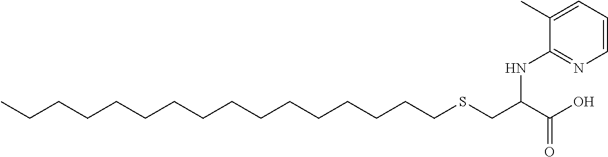 | 436.69 | 3- (hexadecylsulfanyl)- 2-[(3- methylpyridin-2- yl)amino]propanoic acid |
| L-56 | 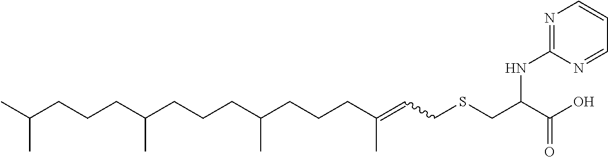 | 477.75 | 2-(pyrimidin-2- ylamino)-3- [(3,7,11,15- tetramethylhexadec- 2- en-1-yl)sulfanyl] propanoic acid |
| L-57 | 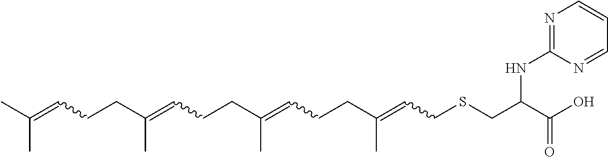 | 471.70 | 2-(pyrimidin-2- ylamino)-3- {[3,7,11,15- tetramethylhexadeca- 2,6,10,14-tetraen-1- yl]sulfanyl} propanoic acid |
| L-58 | 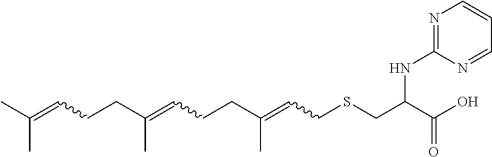 | 403.58 | 2-(pyrimidin-2- ylamino)-3-[(3,7,11- trimethyldodeca- 2,6,10-trien-1- yl)sulfanyl] propanoic acid |
| L-59 | 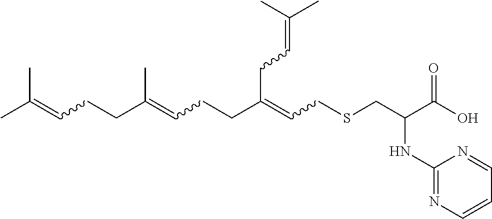 | 457.67 | 3-{[7,11-dimethyl- 3-(3-methylbut-2- en-1-yl)dodeca- 2,6,10-trien-1- yl]sulfanyl}-2- (pyrimidin-2- ylamino) propanoic acid |
| L-60 | 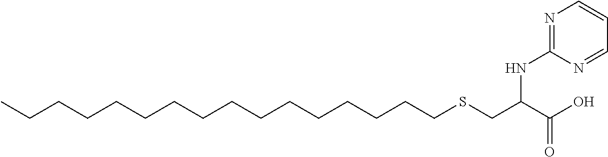 | 423.66 | 3- (hexadecylsulfanyl)- 2-(pyrimidin- 2-ylamino) propanoic acid |
| L-61 | 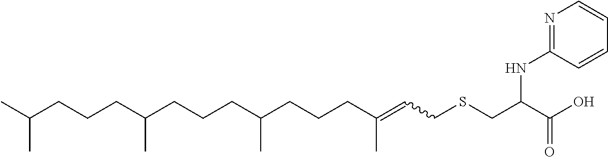 | 476.76 | 2-(pyridin-2- ylamino)-3- [(3,7,11,15- tetramethylhexadec- 2-en-1- yl)sulfanyl] propanoic acid |
| L-62 | 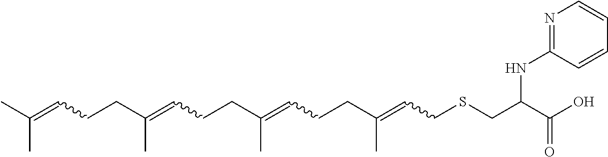 | 470.71 | 2-(pyridin-2- ylamino)-3- {[3,7,11,15- tetramethylhexadeca- 2,6,10,14- tetraen-1-yl] sulfanyl} propanoic acid |
| L-63 | 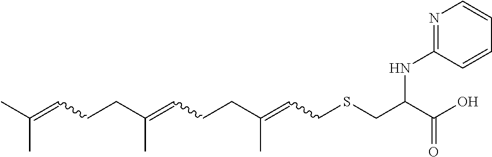 | 402.59 | 2-(pyridin-2- ylamino)-3-[(3,7,11- trimethyldodeca- 2,6,10-trien-1- yl)sulfanyl] propanoic acid |
| L-64 | 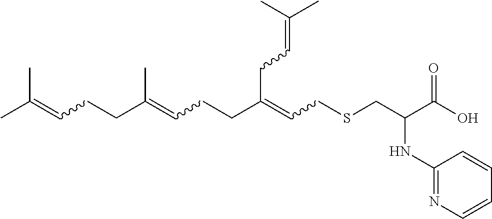 | 456.68 | 3-{[7,11-dimethyl- 3-(3-methylbut-2- en-1-yl)dodeca- 2,6,10-trien-1- yl]sulfanyl}- 2-(pyridin-2- ylamino) propanoic acid |
| L-65 | 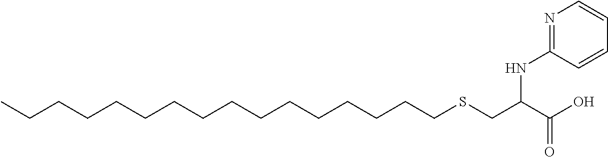 | 422.67 | 3- (hexadecylsulfanyl)-2- (pyridin-2- ylamino) propanoic acid |
| L-66 | 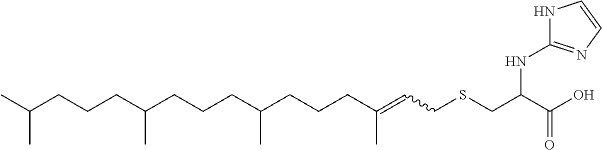 | 465.73 | 2-(1H-imidazol- 2-ylamino)-3- [(3,7,11,15- tetramethylhexadec- 2- en-1-yl)sulfanyl] propanoic acid |
| L-67 | 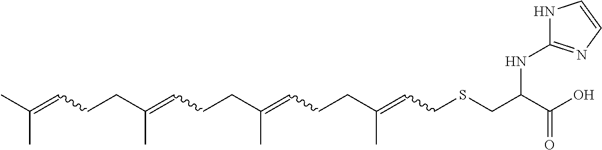 | 459.69 | 2-(1H-imidazol- 2-ylamino)-3- {[3,7,11,15- tetramethylhexadeca- 2,6,10,14-tetraen-1- yl]sulfanyl} propanoic acid |
| L-68 | 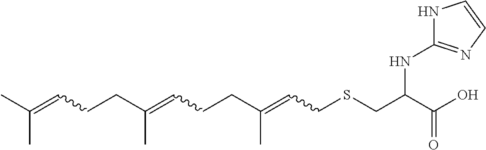 | 391.57 | 2-(1H-imidazol- 2-ylamino)-3- [(3,7,11- trimethyldodeca- 2,6,10- trien-1-yl)sulfanyl] propanoic acid |
| L-69 | 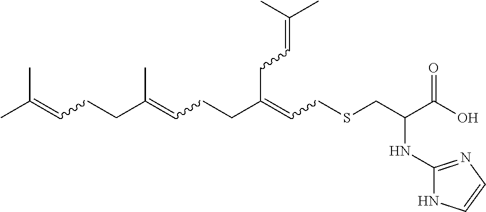 | 445.66 | 3-{[7,11-dimethyl- 3-(3-methylbut-2- en-1-yl)dodeca- 2,6,10-trien-1- yl]sulfanyl}-2- (1H-imidazol-2- ylamino) propanoic acid |
| L-70 | 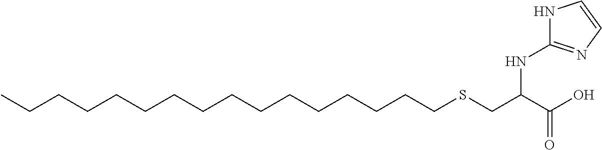 | 411.64 | 3- (hexadecylsulfanyl)- 2-(1H- imidazol-2- ylamino) propanoic acid |
| L-71 | 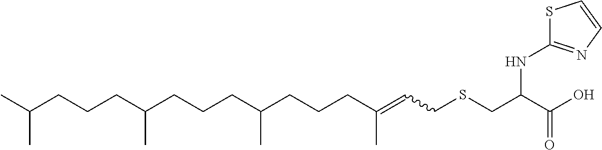 | 482.79 | 3-[(3,7,11,15- tetramethylhexadec- 2- en-1-yl)sulfanyl]- 2-(1,3-thiazol-2- ylamino) propanoic acid |
| L-72 | 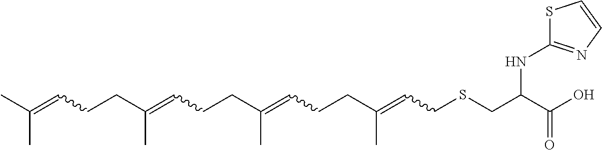 | 476.74 | 3-{[3,7,11,15- tetramethylhexadeca- 2,6,10,14-tetraen- 1-yl]sulfanyl}-2- (1,3-thiazol-2- ylamino)propanoic acid |
| L-73 | 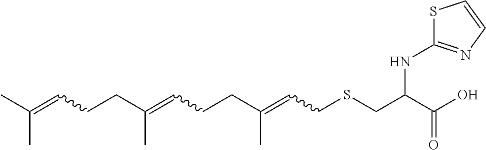 | 408.62 | 2-(1,3-thiazol-2- ylamino)-3- [(3,7,11- trimethyldodeca- 2,6,10- trien-1-yl) sulfanyl] propanoic acid |
| L-74 | 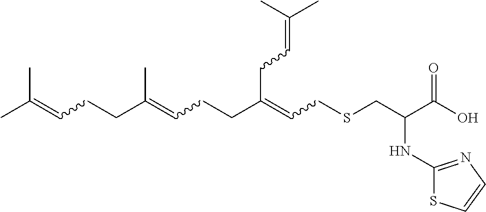 | 462.71 | 3-{[7,11-dimethyl- 3-(3-methylbut-2- en-1-yl)dodeca- 2,6,10-trien-1- yl]sulfanyl}-2- (1,3-thiazol-2- ylamino) propanoic acid |
| L-75 | 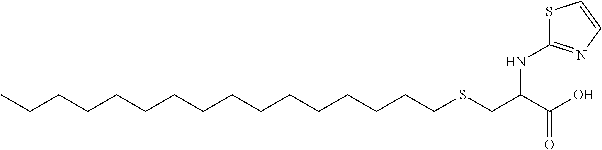 | 428.70 | 3- (hexadecylsulfanyl)- 2-(1,3-thiazol- 2-ylamino) propanoic acid |
| L-76 | 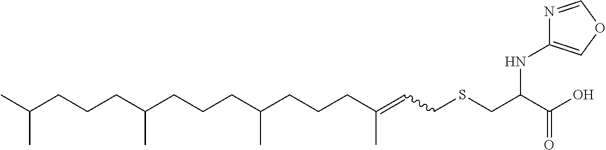 | 466.72 | 2-(1,3-oxazol-4- ylamino)-3- [(3,7,11,15- tetramethylhexadec- 2- en-1-yl)sulfanyl] propanoic acid |
| L-77 | 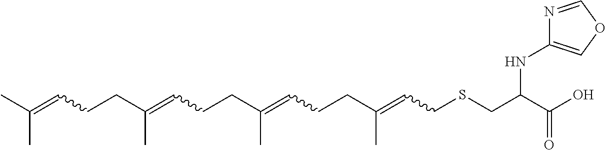 | 460.67 | 2-(1,3-oxazol-4- ylamino)-3- {[3,7,11,15- tetramethylhexadeca- 2,6,10,14-tetraen-1- yl]sulfanyl} propanoic acid |
| L-78 | 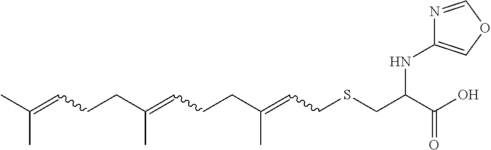 | 392.55 | 2-(1,3-oxazol-4- ylamino)-3- [(3,7,11- trimethyldodeca- 2,6,10-trien-1- yl)sulfanyl] propanoic acid |
| L-79 | 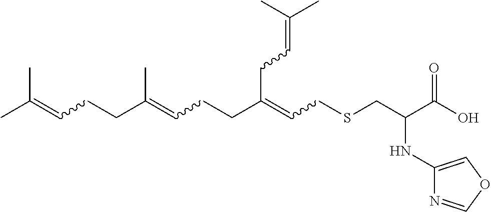 | 446.65 | 3-{[7,11-dimethyl- 3-(3-methylbut-2- en-1-yl)dodeca- 2,6,10-trien-1- yl]sulfanyl}-2- (1,3-oxazol-4- ylamino) propanoic acid |
| L-80 | 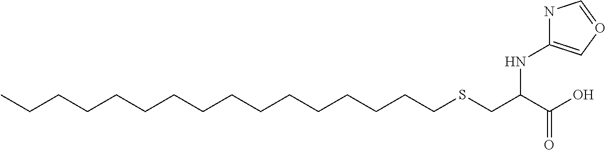 | 412.63 | 3- (hexadecylsulfanyl)- 2-(1,3-oxazol- 4-ylamino) propanoic acid |
| L-81 | 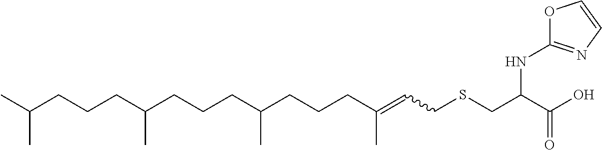 | 466.72 | 2-(1,3-oxazol-2- ylamino)-3- [(3,7,11,15- tetramethylhexadec- 2- en-1-yl)sulfanyl] propanoic acid |
| L-82 | 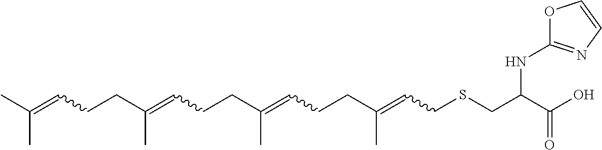 | 460.67 | 2-(1,3-oxazol-2- ylamino)-3- {[3,7,11,15- tetramethylhexadeca- 2,6,10,14-tetraen-1- yl]sulfanyl} propanoic acid |
| L-83 | 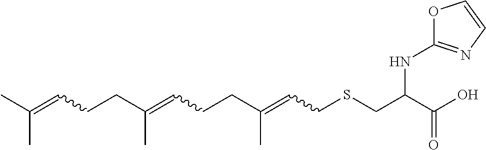 | 392.55 | 2-(1,3-oxazol-2- ylamino)-3- [(3,7,11- trimethyldodeca- 2,6,10-trien-1- yl)sulfanyl] propanoic acid |
| L-84 | 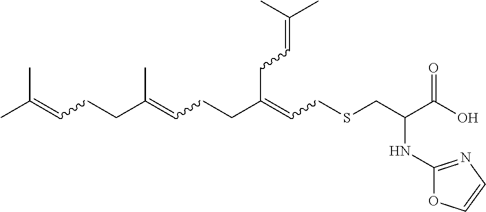 | 446.65 | 3-{[7,11-dimethyl- 3-(3-methylbut-2- en-1-yl)dodeca- 2,6,10-trien-1- yl]sulfanyl}-2- (1,3-oxazol-2- ylamino) propanoic acid |
| L-85 | 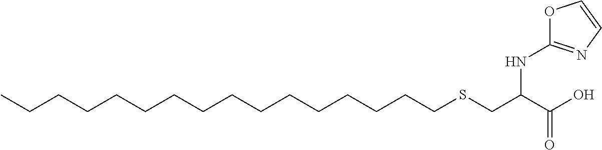 | 412.63 | 3- (hexadecylsulfanyl)- 2-(1,3-oxazol- 2-ylamino) propanoic acid |
| L-86 | 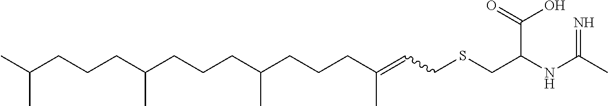 | 440.73 | 2-ethanimidamido- 3-[(3,7,11,15- tetramethylhexadec- 2-en-1- yl)sulfanyl] propanoic acid |
| L-87 | 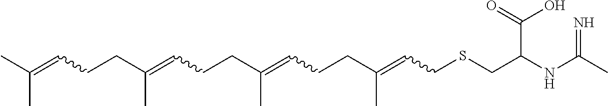 | 434.68 | 2-ethanimidamido- 3-{[3,7,11,15- tetramethylhexadeca- 2,6,10,14- tetraen-1-yl] sulfanyl} propanoic acid |
| L-88 | 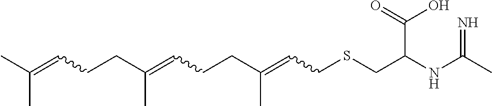 | 366.56 | 2-ethanimidamido- 3-[(3,7,11- trimethyldodeca- 2,6,10-trien-1- yl)sulfanyl] propanoic acid |
| L-89 | 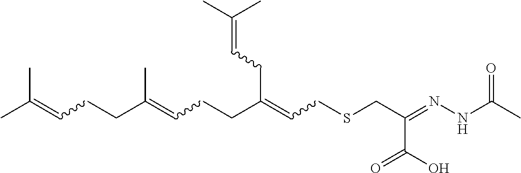 | 420.65 | 3-{[7,11-dimethyl- 3-(3-methylbut-2- en-1-yl)dodeca- 2,6,10-trien-1- yl]sulfanyl}-2- ethanimidamido- propanoic acid |
| L-90 | 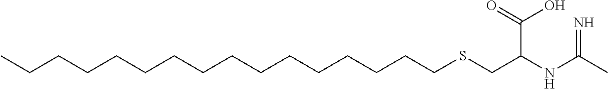 | 386.64 | 2-ethanimidamido-3- (hexadecylsulfanyl) propanoic acid |
| L-91 | 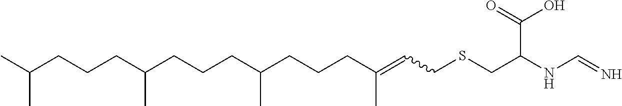 | 426.70 | 2-methanimidamido- 3-[(3,7,11,15- tetramethylhexadec- 2-en-1- yl)sulfanyl] propanoic acid |
| L-92 | 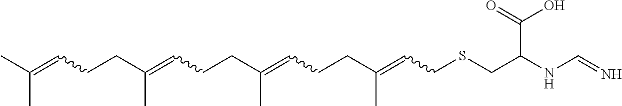 | 420.65 | 2-methanimidamido- 3-{[3,7,11,15- tetramethylhexadeca- 2,6,10,14- tetraen-1-yl]sulfanyl} propanoic acid |
| L-93 | 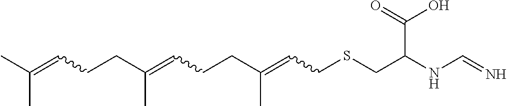 | 352.54 | 2-methanimidamido- 3-[(3,7,11- trimethyldodeca- 2,6,10-trien-1- yl)sulfanyl] propanoic acid |
| L-94 | 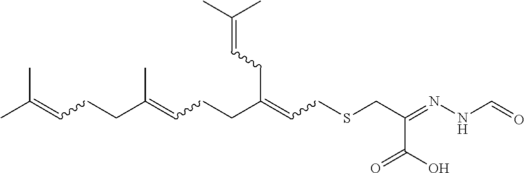 | 406.63 | 3-{[7,11-dimethyl-3- (3-methylbut-2- en-1-yl)dodeca- 2,6,10-trien-1- yl]sulfanyl}-2- methanimidamido- propanoic acid |
| L-95 | 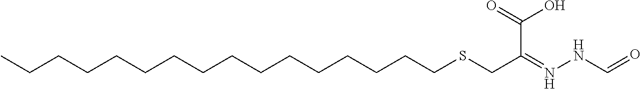 | 372.61 | 3- (hexadecylsulfanyl)- 2- methanimidamido- propanoic acid |
| L-96 | 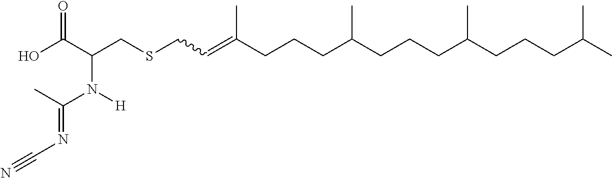 | 465.73 | 2-(N′-cyano- ethanimidamido)- 3- [(3,7,11,15- tetramethylhexadec- 2- en-1-yl)sulfanyl] propanoic acid |
| L-97 | 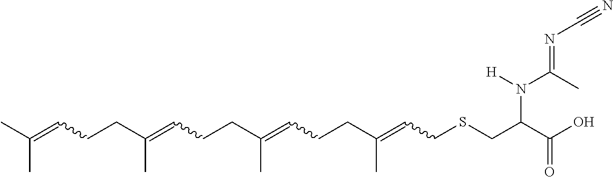 | 459.69 | 2-(N′- cyano- ethanimidamido)-3- {[3,7,11,15- tetramethylhexadeca- 2,6,10,14-tetraen-1- yl]sulfanyl} propanoic acid |
| L-98 | 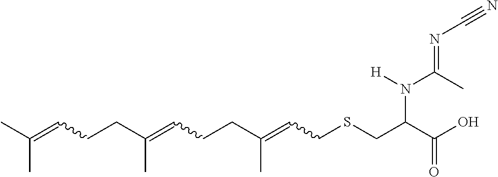 | 391.57 | 2-(N′-cyano- ethanimidamido)-3- [(3,7,11- trimethyldodeca- 2,6,10- trien-1-yl)sulfanyl] propanoic acid |
| L-99 | 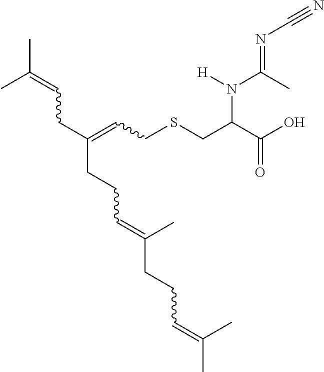 | 445.66 | 2-(N′-cyano- ethanimidamido)-3- {[7,11-dimethyl-3- (3-methylbut-2- en-1-yl)dodeca- 2,6,10-trien-1- yl]sulfanyl} propanoic acid |
| L-100 | 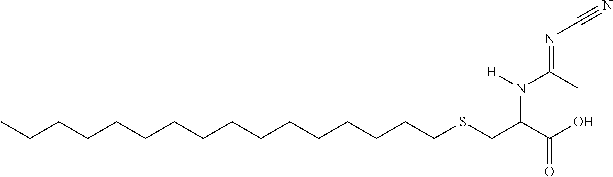 | 411.64 | 2-(N′-cyano- ethanimidamido)-3- (hexadecylsulfanyl) propanoic acid |
| L-101 | 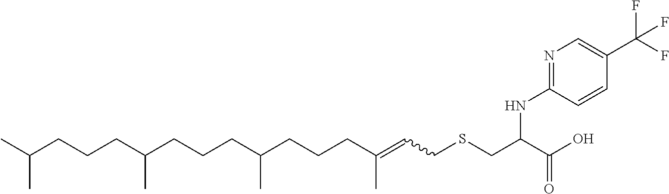 | 544.76 | 3-[(3,7,11,15- tetramethylhexadec- 2- en-1-yl)sulfanyl]- 2-{[5- (trifluoromethyl) pyridin-2- yl]amino} propanoic acid |
| L-102 | 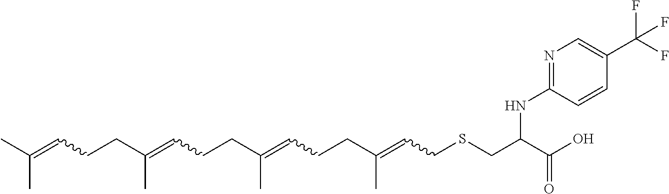 | 538.71 | 3-[(3,7,11,15- tetramethylhexadeca- 2,6,10,14-tetraen-1- yl]sulfanyl}-2- [5-(trifluoromethyl) pyridin-2- yl)amino] propanoic acid |
| L-103 | 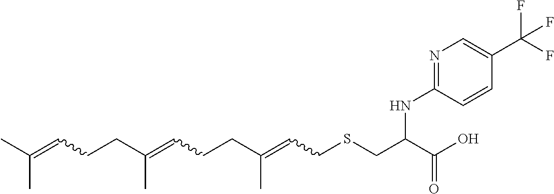 | 470.59 | 2-{[5- (trifluoromethyl) pyridin-2- yl]amino}-3- [(3,7,11- trimethyldodeca- 2,6,10-trien-1- yl)sulfanyl] propanoic acid |
| L-104 | 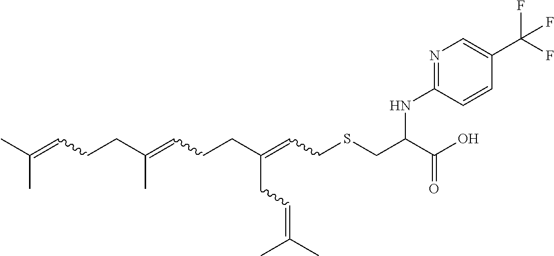 | 524.68 | 3-{[7,11-dimethyl- 3-(3-methylbut-2- en-1-yl)dodeca- 2,6,10-trien-1- yl]sulfanyl}-2-{[5- (trifluoromethyl) pyridin-2- yl]amino} propanoic acid |
| L-105 | 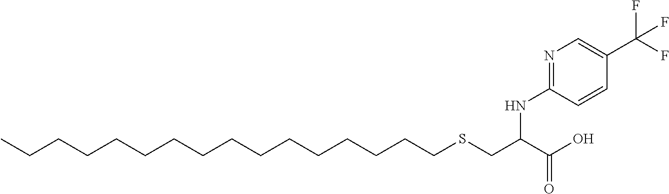 | 490.67 | 3- (hexadecylsulfanyl)- 2-{[5- (trifluoromethyl) pyridin-2- yl]amino} propanoic acid |
| L-106 | 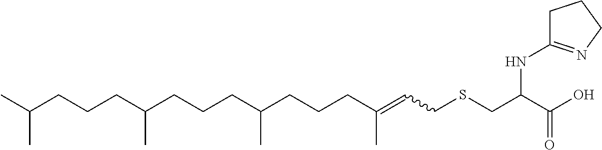 | 466.76 | 2-(3,4-dihydro- 2H-pyrrol-5- ylamino)-3- [(3,7,11,15- tetramethylhexadec- 2-en-1- yl)sulfanyl] propanoic acid |
| L-107 | 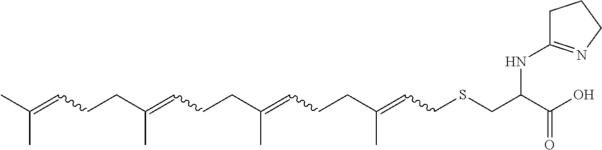 | 460.71 | 2-(3,4-dihydro- 2H-pyrrol-5- ylamino)-3- [(3,7,11,15- tetramethylhexadeca- 2,6,10,14- tetraen-1-yl)sulfanyl] propanoic acid |
| L-108 | 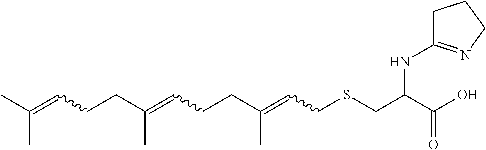 | 392.60 | 2-(3,4-dihydro- 2H-pyrrol-5- ylamino)-3- [(3,7,11- trimethyldodeca- 2,6,10-trien-1- yl)sulfanyl] propanoic acid |
| L-109 | 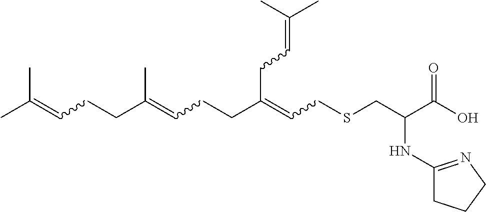 | 446.69 | 2-(3,4-dihydro- 2H-pyrrol-5- ylamino)-3- {[7,11- dimethyl-3-(3- methylbut-2-en-1- yl)dodeca-2,6,10- trien-1-yl]sulfanyl} propanoic acid |
| L-110 | 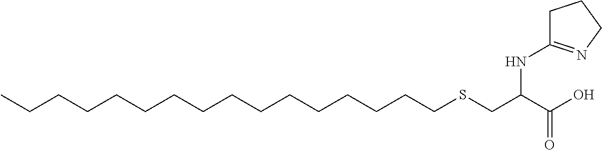 | 412.67 | 2-(3,4-dihydro- 2H-pyrrol-5- ylamino)-3- (hexadecylsulfanyl) propanoic acid |
| L-111 | 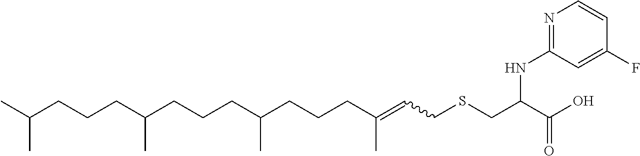 | 494.75 | 2-[(4-fluoropyridin- 2-yl)amino]-3- [(3,7,11,15- tetramethylhexadec- 2- en-1-yl)sulfanyl] propanoic acid |
| L-112 | 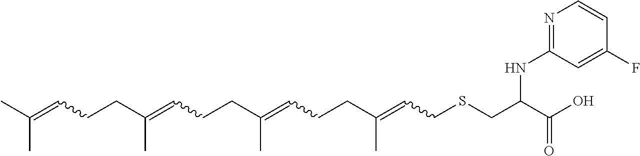 | 488.70 | 2-[(4-fluoropyridin- 2-yl)amino]-3- [(3,7,11,15- tetramethylhexadeca- 2,6,10,14-tetraen-1- yl)sulfanyl] propanoic acid |
| L-113 | 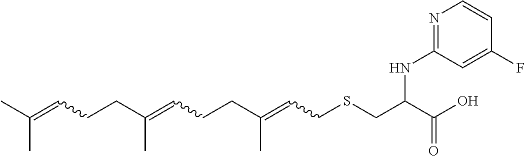 | 420.58 | 2-[(4-fluoropyridin- 2-yl)amino]-3- [(3,7,11- trimethyldodeca- 2,6,10- trien-1-yl)sulfanyl] propanoic acid |
| L-114 | 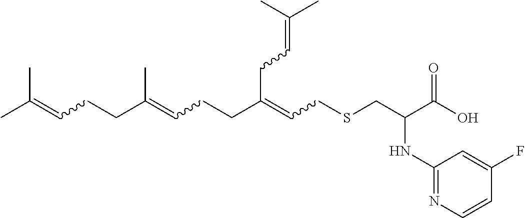 | 474.67 | 3-{[7,11-dimethyl- 3-(3-methylbut-2- en-1-yl)dodeca- 2,6,10-trien-1- yl]sulfanyl}-2- [(4-fluoropyridin-2- yl)amino] propanoic acid |
| L-115 | 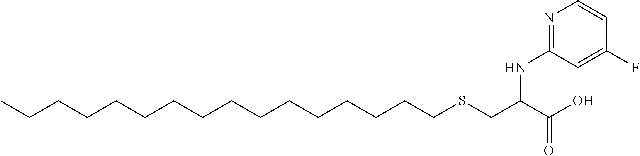 | 440.66 | 2-[(4-fluoropyridin- 2-yl)amino]-3- (hexadecylsulfanyl) propanoic acid |
| L-116 | 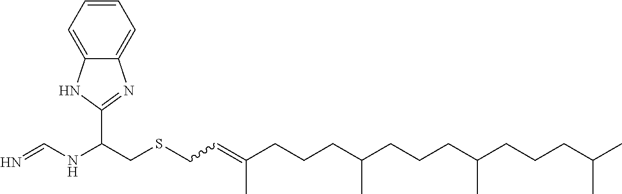 | 498.81 | N-[1-(1H-1,3- benzodiazol- 2-yl)-2- [(3,7,11,15- tetramethylhexadec- 2- en-1- yl)sulfanyl]ethyl] methanimidamide |
| L-117 | 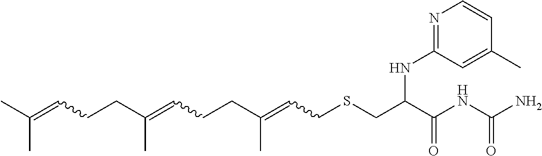 | 458.66 | {2-[(4- methylpyridin-2- yl)amino]-3- [(3,7,11- trimethyldodeca- 2,6,10- trien-1-yl)sulfanyl] propanoyl}urea |
| L-118 | 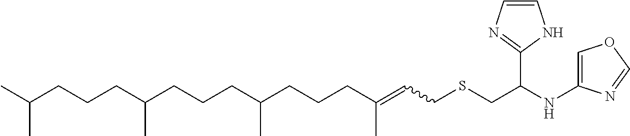 | 488.77 | N-[1-(1H-imidazol- 2-yl)-2- [(3,7,11,15- tetramethylhexadec- 2- en-1-yl)sulfanyl] ethyl]-1,3-oxazol-4- amine |
| L-119 | 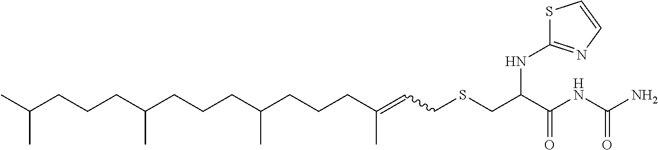 | 524.83 | {3-[(3,7,11,15- tetramethylhexadec- 2-en-1-yl)sulfanyl]- 2-(1,3-thiazol-2- ylamino) propanoyl}urea |
| L-120 | 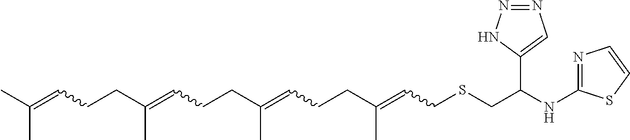 | 499.78 | N-(2-[(3,7,11,15- tetramethylhexadeca- 2,6,10,14- tetraen-1-yl) sulfanyl}-1- (1H-1,2,3 - triazol-5-yl)ethyl)- 1,3-thiazol-2- amine |
| L-121 | 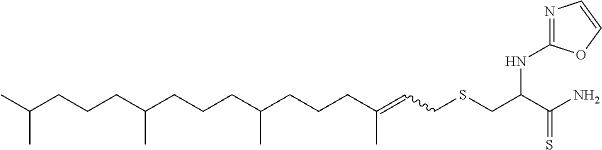 | 481.80 | 2-(1,3-oxazol-2- ylamino)-3- [(3,7,11,15- tetramethylhexadec- 2- en-1-yl)sulfanyl] propanethioamide |
| L-122 | 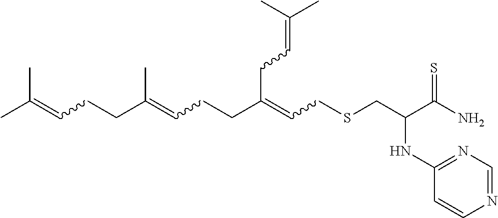 | 472.75 | 3-{[7,11-dimethyl- 3-(3-methylbut-2- en-1-yl)dodeca- 2,6,10-trien-1- yl]sulfanyl}-2- (pyrimidin-4- ylamino) propanethioamide |
| L-123 | 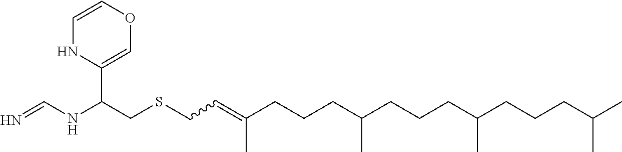 | 463.76 | N-[1-(4H-1,4- oxazin-3-yl)-2- [(3,7,11,15- tetramethylhexadec- 2-en-1- yl)sulfanyl]ethyl] methanimidamide |
| L-124 | 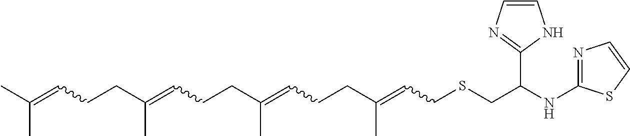 | 498.79 | N-[1-(1H- imidazol-2-yl)-2- {[3,7,11,15- tetramethylhexadeca- 2,6,10,14-tetraen-1- yl]sulfanyl}ethyl]- 1,3-thiazol-2- amine |
| L-125 | 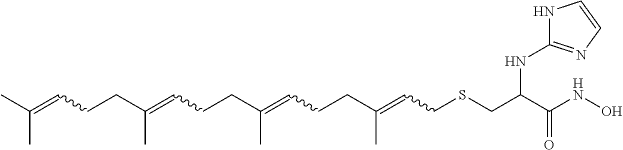 | 474.70 | N-hydroxy-2- (1H-imidazol-2- ylamino)-3- {[3,7,11,15- tetramethylhexadeca- 2,6,10,14- tetraen-1-yl] sulfanyl} propanamide |
| L-126 | 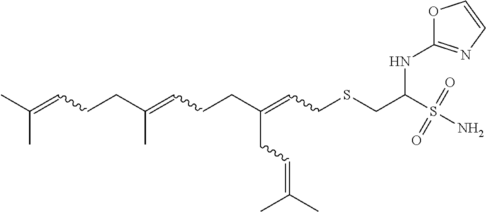 | 481.71 | N-(2-{[7,11- dimethyl-3-(3- methylbut-2-en-1- yl)dodeca-2,6,10- trien-1-yl] sulfanyl}-1- sulfamoylethyl)- 1,3-oxazol-2- amine |
| L-127 | 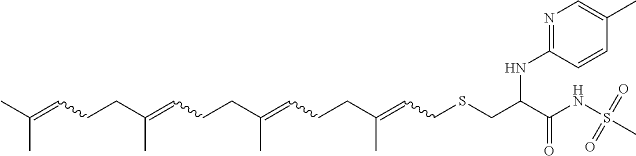 | 561.84 | N- methanesulfonyl- 2-[(5- methylpyridin-2- yl)amino]-3- {[3,7,11,15- tetramethylhexadeca- 2,6,10,14-tetraen-1- yl]sulfanyl} propanamide |
| L-128 | 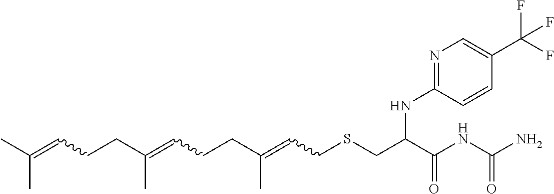 | 512.63 | (2-{[5- (trifluoromethyl) pyridin-2- yl]amino}-3- [(3,7,11- trimethyldodeca- 2,6,10-trien-1- yl)sulfanyl] propanoyl)urea |
| L-129 | 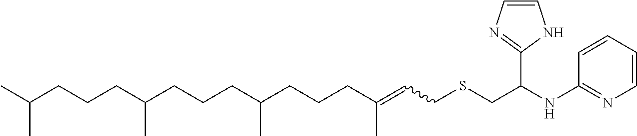 | 498.81 | N-[1-(1H- imidazol-2-yl)-2- [(3,7,11,15- tetramethylhexadec- 2- en-1-yl)sulfanyl] ethyl]pyridin-2- amine |
| L-130 | 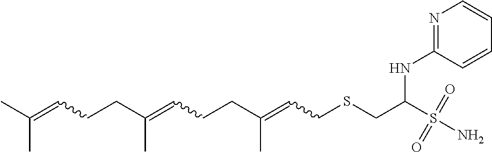 | 437.66 | N-{1-sulfamoyl-2- [(3,7,11- trimethyldodeca- 2,6,10-trien-1- yl)sulfanyl]ethyl} pyridin-2-amine |
| L-131 | 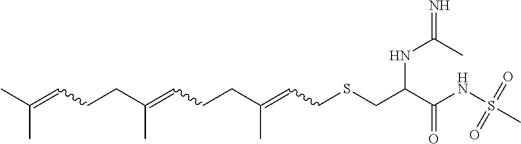 | 443.67 | 2-ethanimidamido- N- methanesulfonyl-3- [(3,7,11- trimethyldodeca- 2,6,10-trien-1- yl)sulfanyl] propanamide |
| L-132 | 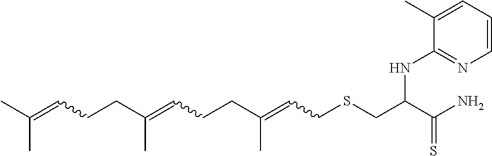 | 431.70 | 2-[(3-methylpyridin- 2-yl)amino]-3- [(3,7,11- trimethyldodeca- 2,6,10- trien-1-yl)sulfanyl] propanethioamide |
| L-133 | 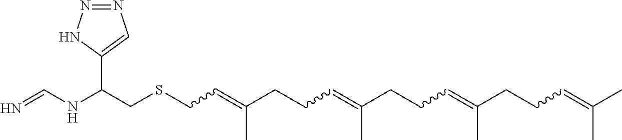 | 443.69 | N-(2-{[3,7,11,15- tetramethylhexadeca- 2,6,10,14- tetraen-1-yl] sulfanyl}-1- (1H-1,2,3- triazol-5-yl)ethyl) methanimidamide |
| L-134 | 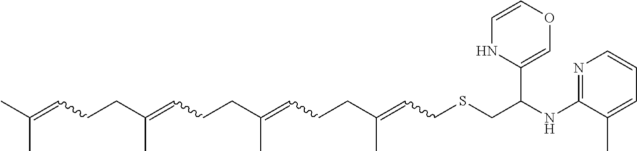 | 521.80 | 3-methyl-N-[1- (4H-1,4-oxazin-3-yl)- 2-{[3,7,11,15- tetramethylhexadeca- 2,6,10,14-tetraen-1- yl]sulfanyl}ethyl] pyridin-2-amine |
| L-135 | 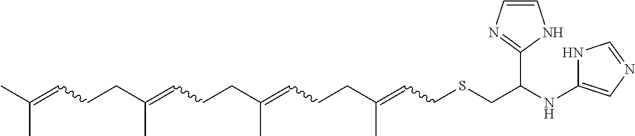 | 481.74 | N-[1-(1H- imidazol-2-yl)-2- {[3,7,11,15- tetramethylhexadeca- 2,6,10,14-tetraen-1- yl]sulfanyl}ethyl]- 1H-imidazol-5- amine |
| L-136 | 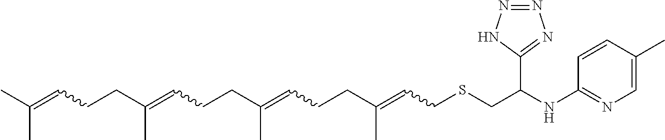 | 508.77 | 5-methyl-N-(2- {[3,7,11,15- tetramethylhexadeca- 2,6,10,14- tetraen-1-yl] sulfanyl}-1-(1H- 1,2,3,4- tetrazol-5-yl)ethyl) pyridin-2-amine |
| L-137 | 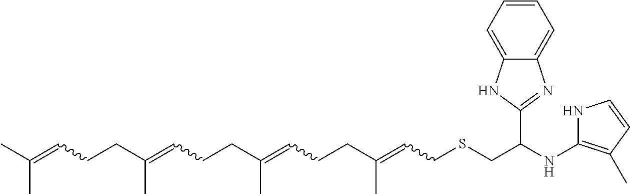 | 544.84 | N-[1-(1H-1,3- benzodiazol-2-yl)-2- {[3,7,11,15- tetramethylhexadeca- 2,6,10,14-tetraen-1- yl]sulfanyl}ethyl]- 3-methyl-1H- pyrrol-2-amine |
| L-138 | 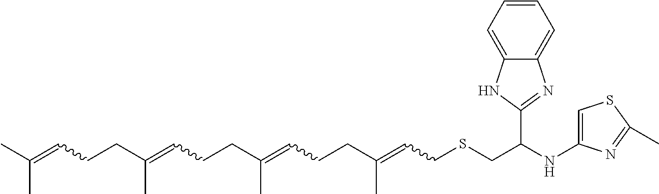 | 562.88 | N-[1-(1H-1,3- benzodiazol-2-yl)-2- {[3,7,11,15- tetramethylhexadeca- 2,6,10,14-tetraen-1- yl]sulfanyl}ethyl]- 2-methyl-1,3- thiazol-4-amine |
| L-139 | 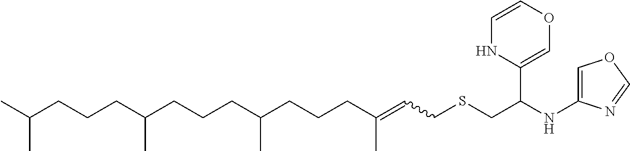 | 503.78 | N-[1-(4H-1,4- oxazin-3-yl)-2- [(3,7,11,15- tetramethylhexadec-2- en-1-yl)sulfanyl] ethyl]-1,3-oxazol-4- amine |
| L-140 | 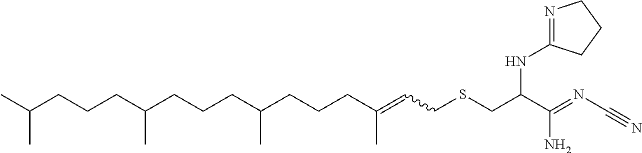 | 489.80 | N′-cyano-2-(3,4- dihydro-2H-pyrrol- 5-ylamino)-3- [(3,7,11,15- tetramethylhexadec- 2-en-1- yl)sulfanyl] propanimidamide |
| L-141 | 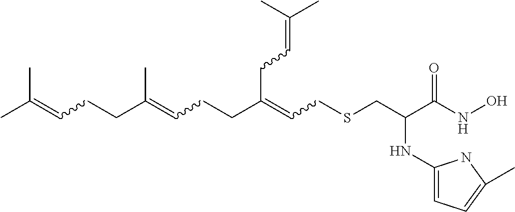 | 473.31 | 3-{[7,11-dimethyl- 3-(3-methylbut-2- en-1-yl)dodeca- 2,6,10-trien-1- yl]sulfanyl}-N- hydroxy-2-[(5- methyl-1H- pyrrol-2- yl)amino] propanamide |
| L-142 | 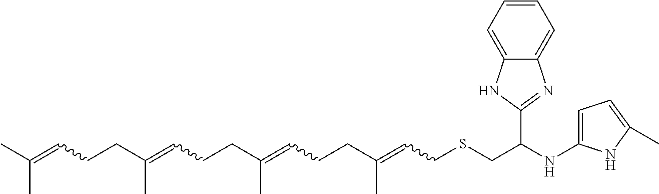 | 544.84 | N-[1-(1H-1,3- benzodiazol-2-yl)-2- {[3,7,11,15- tetramethylhexadeca- 2,6,10,14-tetraen-1- yl]sulfanyl}ethyl]- 5-methyl-1H- pyrrol-2-amine |
| L-143 | 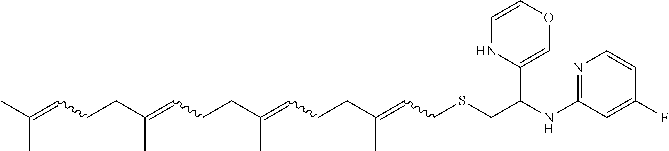 | 525.76 | 4-fluoro-N-[1- (4H-1,4-oxazin-3-yl)- 2-{[3,7,11,15- tetramethylhexadeca- 2,6,10,14-tetraen-1- yl]sulfanyl}ethyl] pyridin-2-amine |
| L-144 | 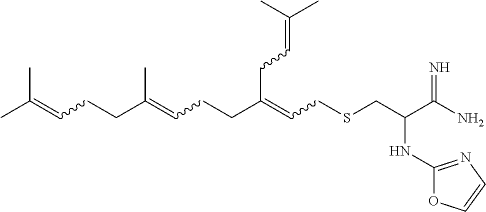 | 444.68 | 3-{[7,11-dimethyl- 3-(3-methylbut-2- en-1-yl)dodeca- 2,6,10-trien-1- yl]sulfanyl}-2- (1,3 -oxazol-2- ylamino) propanimidamide |
| L-145 | 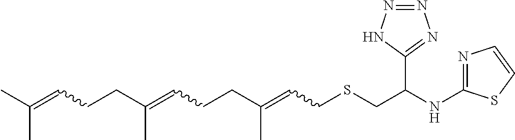 | 431.66 | N-[1-(1H-1,2,3- triazol-5-yl)-2- [(3,7,11- trimethyldodeca- 2,6,10- trien-1-yl) sulfanyl] ethyl]-1,3- thiazol-2-amine |
| L-146 | 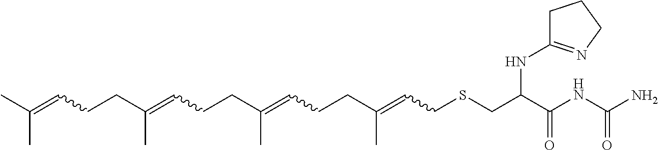 | 502.76 | [2-(3,4-dihydro- 2H-pyrrol-5- ylamino)-3- {[3,7,11,15- tetramethylhexadeca- 2,6,10,14- tetraen-1-yl] sulfanyl} propanoyl]urea |
| L-147 | 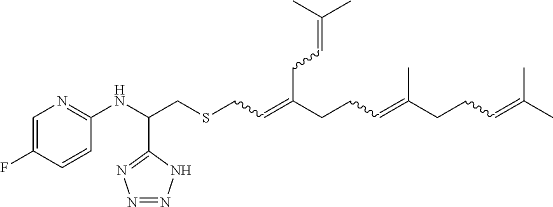 | 498.70 | N-(2-{[7,11- dimethyl-3-(3- methylbut-2-en-1- yl)dodeca-2,6,10- trien-1-yl]sulfanyl}- 1-(1H-1,2,3,4- tetrazol-5-yl)ethyl)- 5-fluoropyridin- 2-amine |
| L-148 | 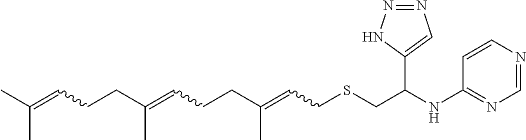 | 426.62 | N-[1-(1H-1,2,3- triazol-5-yl)-2- [(3,7,11- trimethyldodeca- 2,6,10- trien-1-yl)sulfanyl] ethyl]pyrimidin- 4-amine |
| L-149 | 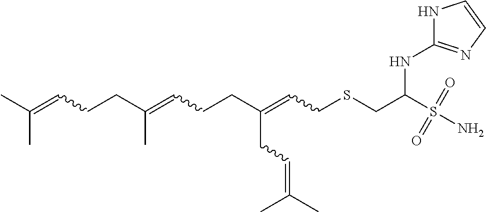 | 480.73 | N-(2-{[7,11- dimethyl-3-(3- methylbut-2-en- 1-yl)dodeca-2,6,10- trien-1-yl] sulfanyl}-1- sulfamoylethyl)- 1H-imidazol-2- amine |
| L-150 | 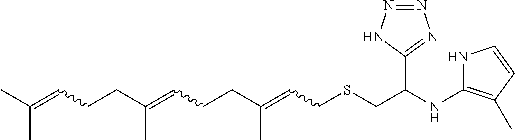 | 428.64 | 3-methyl-N-[1- (1H-1,2,3,4-tetrazol- 5-yl)-2-[(3,7,11- trimethyldodeca- 2,6,10-trien-1-yl) sulfanyl]ethyl]-1H- pyrrol-2-amine |
| L-151 | 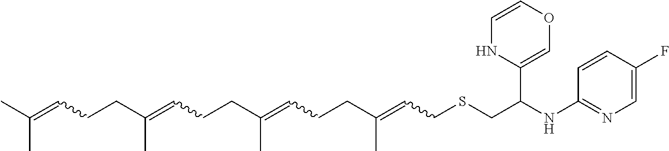 | 525.76 | 5-fluoro-N-[1- (4H-1,4-oxazin-3-yl)- 2-{[3,7,11,15- tetramethylhexadeca- 2,6,10,14-tetraen-1- yl]sulfanyl}ethyl] pyridin-2-amine |
| L-152 | 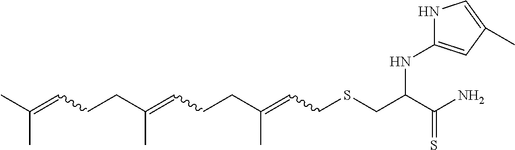 | 419.69 | 2-[(4-methyl-1H- pyrrol-2-yl)amino]- 3-[(3,7,11- trimethyldodeca- 2,6,10- trien-1-yl)sulfanyl] propanethioamide |
| L-153 | 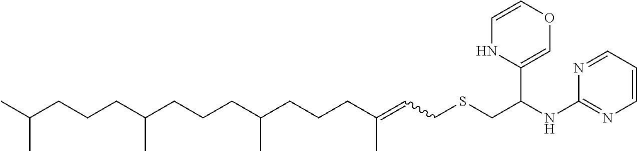 | 514.81 | N-[1-(4H-1,4- oxazin-3-yl)-2- [(3,7,11,15- tetramethylhexadec- 2- en-1-yl)sulfanyl] ethyl]pyrimidin-2- amine |
| L-154 | 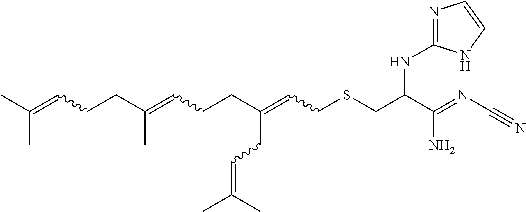 | 468.70 | N′-cyano-3- {[7,11- dimethyl-3-(3- methylbut-2-en- 1-yl)dodeca-2,6,10- trien-1-yl]sulfanyl}- 2-(1H-imidazol- 2-ylamino) propanimidamide |
| L-155 | 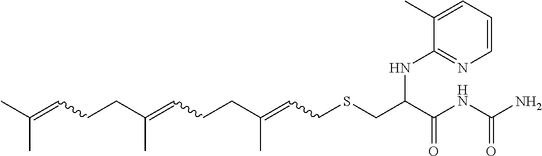 | 458.66 | {2-[(3- methylpyridin-2- yl)amino]-3- [(3,7,11- trimethyldodeca- 2,6,10- trien-1-yl)sulfanyl] propanoyl}urea |
| L-156 | 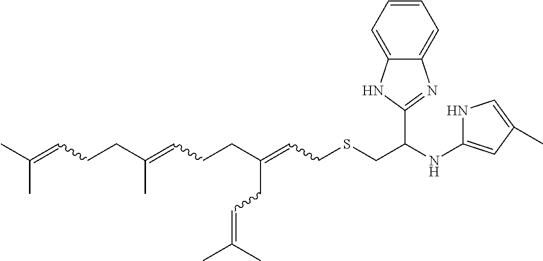 | 530.81 | N-[1-(1H-1,3- benzodiazol-2-yl)-2- {[7,11-dimethyl- 3-(3-methylbut-2- en-1-yl)dodeca- 2,6,10-trien-1- yl]sulfanyl}ethyl]- 4-methyl-1H- pyrrol-2-amine |
| L-157 | 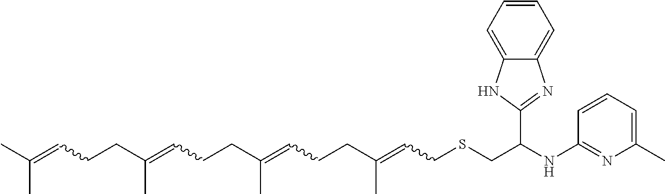 | 556.85 | N-[1-(1H-1,3- benzodiazol-2-yl)-2- {[3,7,11,15- tetramethylhexadeca- 2,6,10,14-tetraen-1- yl]sulfanyl}ethyl]- 6-methylpyridin- 2-amine |
| L-158 | 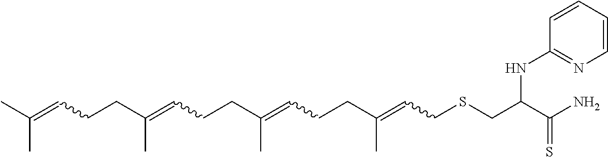 | 485.79 | 2-(pyridin-2- ylamino)-3- {[3,7,11,15- tetramethylhexadeca- 2,6,10,14- tetraen-1- yl]sulfanyl} propanethioamide |
| L-159 | 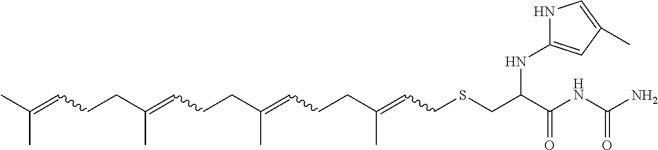 | 514.77 | {2-[(4-methyl- 1H-pyrrol-2- yl)amino]-3- {[3,7,11,15- tetramethylhexadeca- 2,6,10,14- tetraen-1-yl] sulfanyl} propanoyl}urea |
| L-160 | 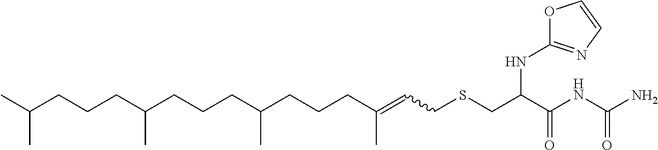 | 508.76 | [2-(1,3-oxazol- 2-ylamino)-3- [(3,7,11,15- tetramethylhexadec- 2- en-1-yl)sulfanyl] propanoyl]urea |
| L-161 | 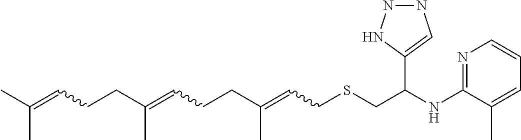 | 439.66 | 3-methyl-N-[1- (1H-1,2,3-triazol-5- yl)-2-[(3,7,11- trimethyldodeca- 2,6,10-trien-1- yl)sulfanyl]ethyl] pyridin-2-amine |
| L-162 | 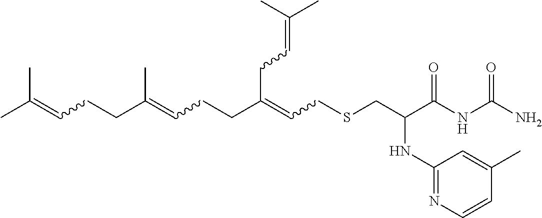 | 512.75 | (3-{[7,11- dimethyl-3-(3- methylbut- 2-en-1-yl) dodeca-2,6,10- trien-1- yl]sulfanyl}-2- [(4- methylpyridin-2- yl)amino] propanoyl)urea |
| L-163 | 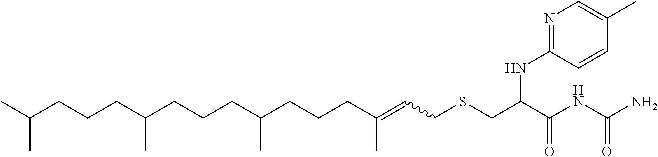 | 532.82 | {2-[(5- methylpyridin-2- yl)amino]-3- [(3,7,11,15- tetramethylhexadec- 2- en-1-yl)sulfanyl] propanoyl}urea |
| L-164 | 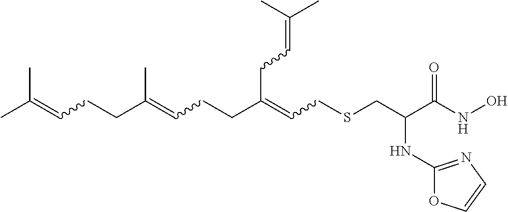 | 461.66 | 3-{[7,11-dimethyl- 3-(3-methylbut-2- en-1-yl)dodeca- 2,6,10-trien-1- yl]sulfanyl}-N- hydroxy-2-(1,3- oxazol-2-ylamino) propanamide |
| L-165 | 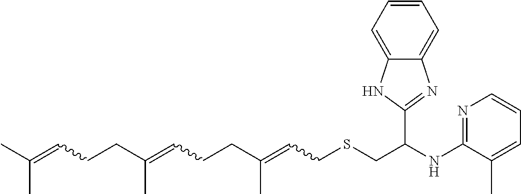 | 488.73 | N-[1-(1H-1,3- benzodiazol- 2-yl)-2- [(3,7,11- trimethyldodeca- 2,6,10- trien-1-yl) sulfanyl] ethyl]-3- methylpyridin- 2-amine |
| L-166 | 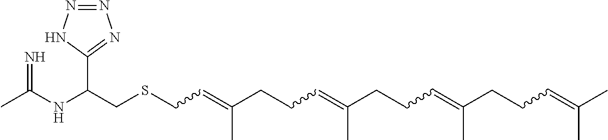 | 458.71 | N-(2-{[3,7,11,15- tetramethylhexadeca- 2,6,10,14- tetraen-1-yl] sulfanyl}-1- (1H-1,2,3,4- tetrazol-5-yl)ethyl) ethanimidamide |
| L-167 | 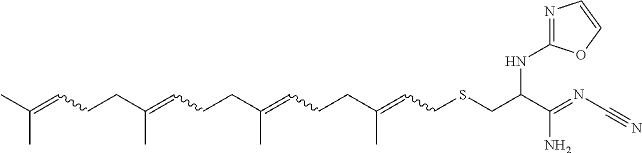 | 483.71 | N′-cyano-2-(1,3- oxazol-2-ylamino)- 3-{[3,7,11,15- tetramethylhexadeca- 2,6,10,14-tetraen-1- yl]sulfanyl} propanimidamide |
| L-168 | 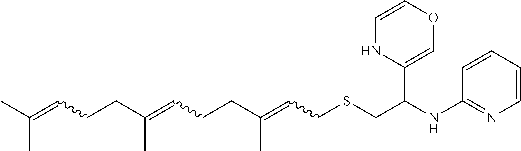 | 439.66 | N-[1-(4H-1,4- oxazin-3-yl)-2- [(3,7,11- trimethyldodeca- 2,6,10- trien-1-yl) sulfanyl]ethyl] pyridin-2- amine |
| L-169 | 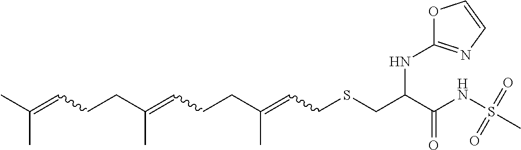 | 469.66 | N-methanesulfonyl- 2-(1,3-oxazol-2- ylamino)-3-[(3,7,11- trimethyldodeca- 2,6,10-trien-1- yl)sulfanyl] propanamide |
| L-170 | 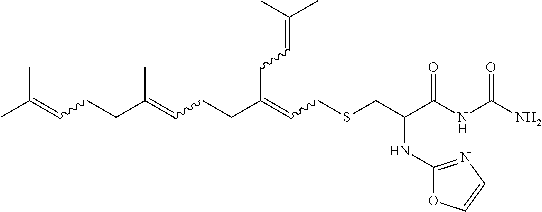 | 488.69 | (3-{[7,11- dimethyl-3-(3- methylbut- 2-en-1-yl)dodeca- 2,6,10-trien-1- yl]sulfanyl}-2- (1,3-oxazol-2- ylamino) propanoyl)urea |
| L-171 | 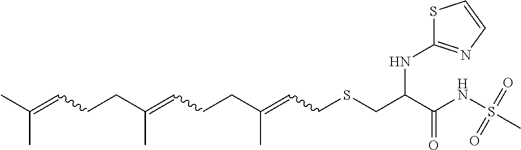 | 485.73 | N-methanesulfonyl- 2-(1,3-thiazol-2- ylamino)-3- [(3,7,11- trimethyldodeca- 2,6,10-trien-1- yl)sulfanyl] propanamide |
| L-172 | 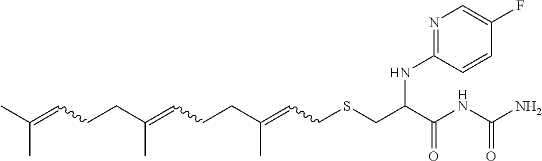 | 462.62 | {2-[(5- fluoropyridin-2- yl)amino]-3- [(3,7,11- trimethyldodeca- 2,6,10- trien-1-yl) sulfanyl] propanoyl}urea |
| L-173 | 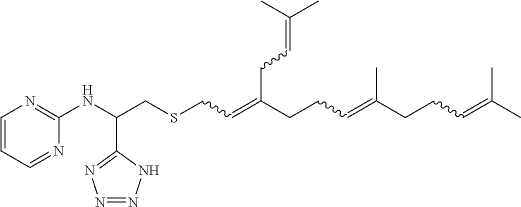 | 481.70 | N-(2-{[7,11- dimethyl-3-(3- methylbut-2-en- 1-yl)dodeca-2,6,10- trien-1-yl] sulfanyl}-1- (1H-1,2,3,4- tetrazol-5-yl) ethyl)pyrimidin-2- amine |
| L-174 | 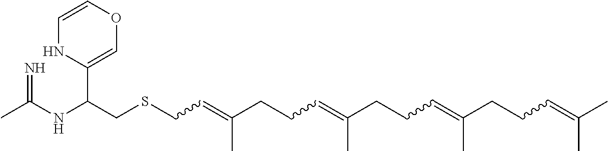 | 471.74 | N-[1-(4H-1,4- oxazin-3-yl)-2- {[3,7,11,15- tetramethylhexadeca- 2,6,10,14-tetraen-1- yl]sulfanyl}ethyl] ethanimidamide |
| L-175 | 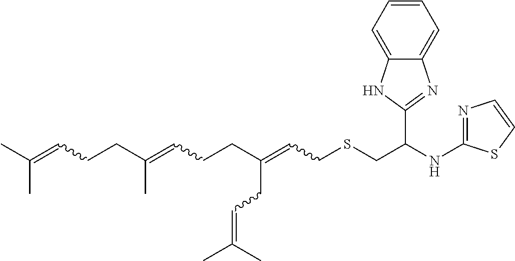 | 534.82 | N-[1-(1H-1,3- benzodiazol- 2-yl)-2- {[7,11-dimethyl- 3-(3-methylbut-2- en-1-yl)dodeca- 2,6,10-trien-1- yl]sulfanyl}ethyl]- 1,3-thiazol-2- amine |
| L-176 | 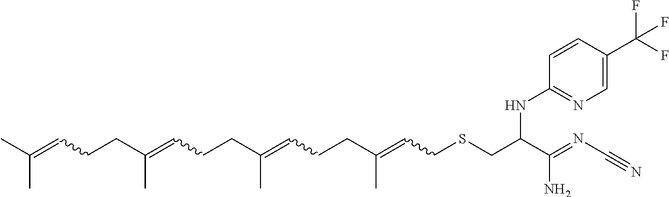 | 561.75 | N′-cyano-3- {[3,7,11,15- tetramethylhexadeca- 2,6,10,14- tetraen-1-yl] sulfanyl}-2-{[5- (trifluoromethyl) pyridin-2- yl]amino) propanimidamide |
| L-177 | 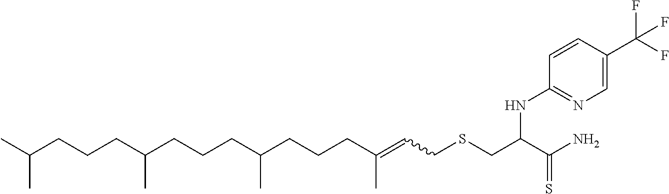 | 559.84 | 3-[(3,7,11,15- tetramethylhexadec- 2- en-1-yl)sulfanyl]- 2-{[5- (trifluoromethyl) pyridin-2- yl]amino) propanethioamide |
| L-178 | 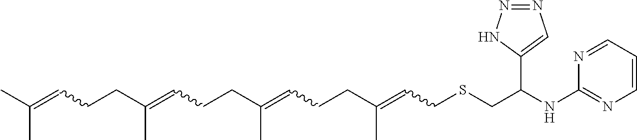 | 495.73 | N-(2-{[3,7,11,15- tetramethylhexadeca- 2,6,10,14- tetraen-1-yl] sulfanyl}-1-(1H- 1,2,3,4- tetrazol-5-yl) ethyl)pyrimidin-2- amine |
| L-179 | 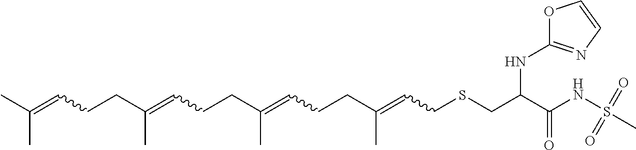 | 537.78 | N-methanesulfonyl- 2-(1,3-oxazo1-2- ylamino)-3- {[3,7,11,15- tetramethylhexadeca- 2,6,10,14- tetraen-1-yl] sulfanyl}propanamide |
| L-180 | 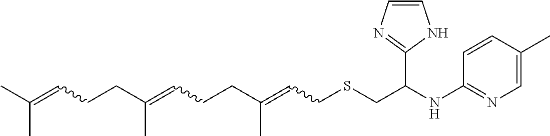 | 438.67 | N-[1-(1H-imidazol- 2-yl)-2-[(3,7,11- trimethyldodeca- 2,6,10-trien-1- yl)sulfanyl]ethyl]- 5-methylpyridin-2- amine |
| L-181 | 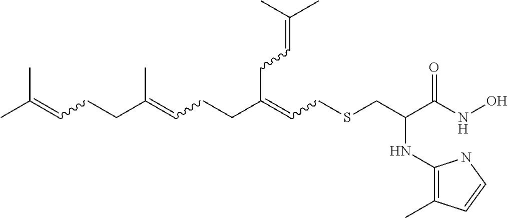 | 473.71 | 3-{[7,11-dimethyl- 3-(3-methylbut-2- en-1-yl)dodeca- 2,6,10-trien-1- yl]sulfanyl}-N- hydroxy-2-[(3- methyl-1H- pyrrol-2- yl)amino] propanamide |
| L-182 | 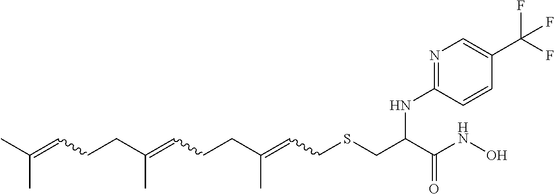 | 485.61 | N-hydroxy-2-{[5- (trifluoromethyl) pyridin-2- yl]amino}-3- [(3,7,11- trimethyldodeca- 2,6,10-trien-1- yl)sulfanyl] propanamide |
| L-183 | 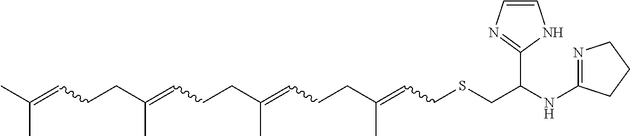 | 482.77 | N-[1-(1H- imidazol-2-yl)-2- {[3,7,11,15- tetramethylhexadeca- 2,6,10,14-tetraen-1- yl]sulfanyl}ethyl]- 3,4-dihydro-2H- pyrrol-5-amine |
| L-184 | 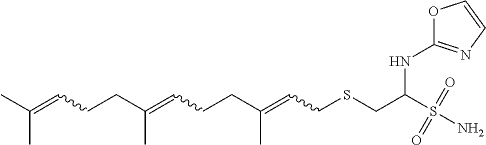 | 427.62 | N-{1-sulfamoyl- 2-[(3,7,11- trimethyldodeca- 2,6,10-trien-1- yl)sulfanyl]ethyl}- 1,3-oxazol-2- amine |
| L-185 | 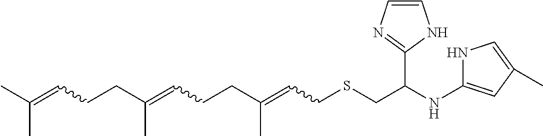 | 426.66 | N-[1-(1H- imidazol-2-yl)-2- [(3,7,11- trimethyldodeca- 2,6,10-trien-1- yl)sulfanyl]ethyl]- 4-methyl-1H- pyrrol-2-amine |
| L-186 | 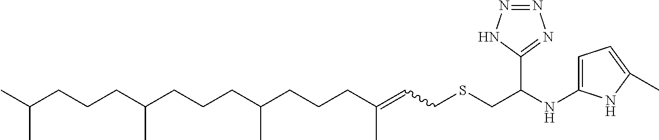 | 502.80 | 5-methyl-N-{2- [(3,7,11,15- tetramethylhexadec- 2-en-1- yl)sulfanyl]-1-(1H- 1,2,3,4-tetrazol- 5-yl)ethyl}-1H- pyrrol-2-amine |
| L-187 | 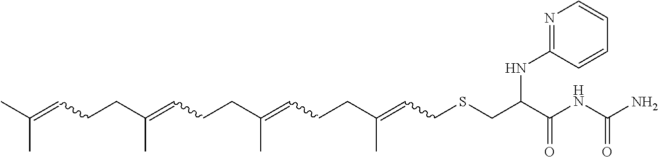 | 512.75 | [2-(pyridin-2- ylamino)-3- {[3,7,11,15- tetramethylhexadeca- 2,6,10,14-tetraen-1- yl]sulfanyl} propanoyl]urea |
| L-188 | 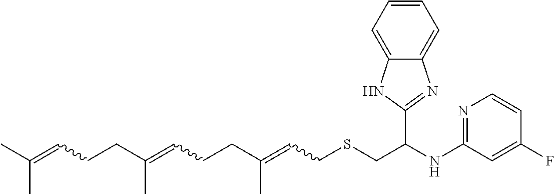 | 492.69 | N-[1-(1H-1,3- benzodiazol-2-yl)-2- [(3,7,11- trimethyldodeca- 2,6,10- trien-1-yl) sulfanyl]ethyl]-4- fluoropyridin- 2-amine |
| L-189 | 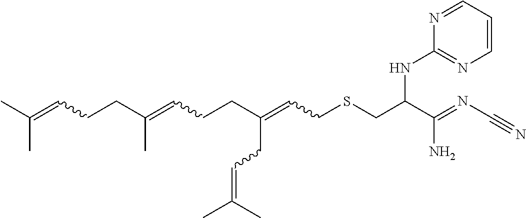 | 480.71 | N′-cyano-3- {[7,11- dimethyl-3-(3- methylbut-2-en-1- yl)dodeca-2,6,10- trien-1-yl] sulfanyl}-2- (pyrimidin-2- ylamino) propanimidamide |
| L-190 | 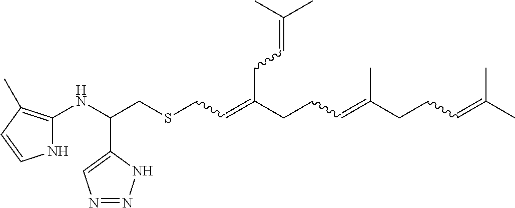 | 481.74 | N-(2-{[7,11- dimethyl-3-(3- methylbut-2-en- 1-yl)dodeca-2,6,10- trien-1-yl]sulfanyl}- 1-(1H-1,2,3- triazol-5-yl)ethyl)- 3-methyl-1H- pyrrol-2-amine |
| L-191 | 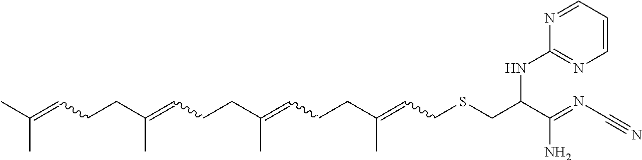 | 494.74 | N′-cyano-2- (pyrimidin-2-ylamino)- 3-{[3,7,11,15- tetramethylhexadeca- 2,6,10,14-tetraen-1- yl]sulfanyl} propanimidamide |
| L-192 | 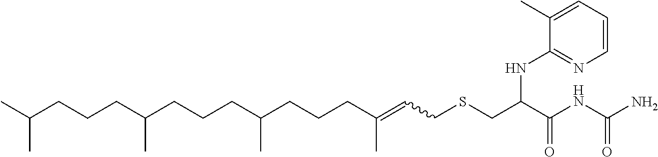 | 532.82 | {2-[(3- methylpyridin-2- yl)amino]-3- [(3,7,11,15- tetramethylhexadec- 2- en-1-yl)sulfanyl] propanoyl}urea |
| L-193 | 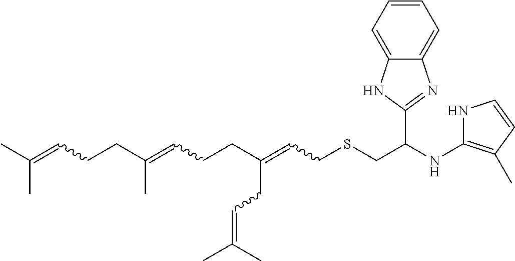 | 530.81 | N-[1-(1H-1,3- benzodiazol-2-yl)-2- {[7,11-dimethyl-3- (3-methylbut-2- en-1-yl)dodeca- 2,6,10-trien-1- yl]sulfanyl}ethyl]- 3-methyl-1H- pyrrol-2-amine |
| L-194 | 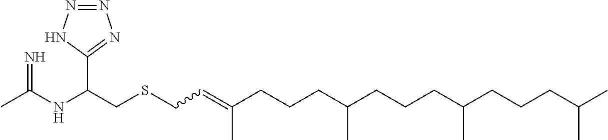 | 464.75 | N-{2-[(3,7,11,15- tetramethylhexadec- 2-en-1- yl)sulfanyl]-1- (1H-1,2,3,4-tetrazol- 5-yl)ethyl} ethanimidamide |
| L-195 | 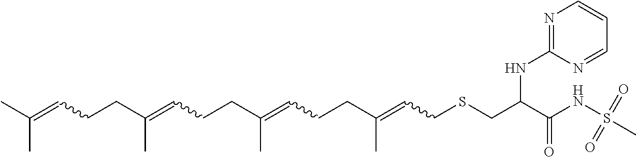 | 548.80 | N- methanesulfonyl- 2-(pyrimidin-2- ylamino)-3- {[3,7,11,15- tetramethylhexadeca- 2,6,10,14- tetraen-1-yl] sulfanyl} propanamide |
| L-196 | 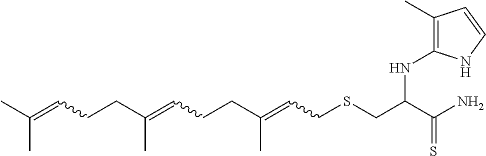 | 419.69 | 2-[(3-methyl-1H- pyrrol-2-yl)amino]- 3-[(3,7,11- trimethyldodeca- 2,6,10- trien-1-yl)sulfanyl] propanethioamide |
| L-197 | 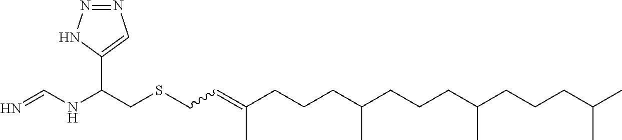 | 449.74 | N-{2-[(3,7,11,15- tetramethylhexadec- 2-en-1- yl)sulfanyl]-1-(1H- 1,2,3-triazol-5- yl)ethyl} methanimidamide |
| L-198 | 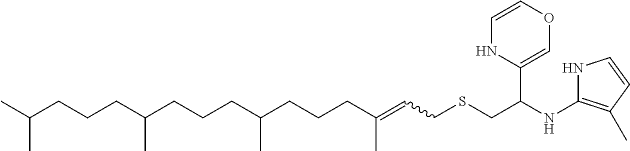 | 515.84 | 3-methyl-N-[1- (4H-1,4- oxazin-3-yl)- 2-[(3,7,11,15- tetramethylhexadec- 2- en-1-yl)sulfanyl] ethyl]-1H-pyrrol-2- amine |
| L-199 | 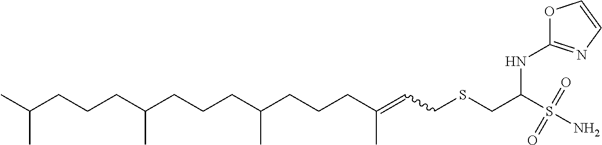 | 501.79 | N-{1-sulfamoyl-2- [(3,7,11,15- tetramethylhexadec- 2-en-1- yl)sulfanyl]ethyl}- 1,3-oxazol-2- amine |
| L-200 | 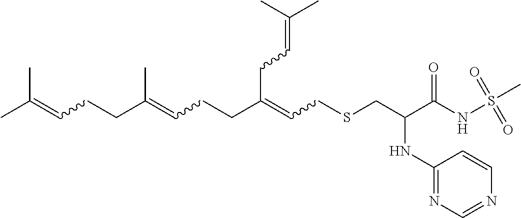 | 534.78 | 3-{[7,11-dimethyl- 3-(3-methylbut-2- en-1-yl)dodeca- 2,6,10-trien-1- yl]sulfanyl}-N- methanesulfonyl-2- (pyrimidin-4- ylamino) propanamide |
| L-201 | 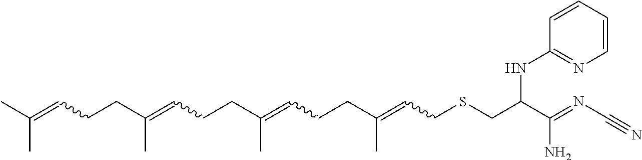 | 493.75 | N′-cyano-2- (pyridin-2- ylamino)-3- {[3,7,11,15- tetramethylhexadeca- 2,6,10,14-tetraen-1- yl]sulfanyl} propanimidamide |
| L-202 | 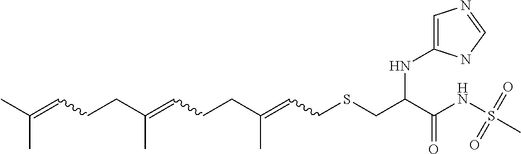 | 468.68 | 2-(1H-imidazol- 5-ylamino)-N- methanesulfonyl- 3-[(3,7,11- trimethyldodeca- 2,6,10-trien-1- yl)sulfanyl] propanamide |
| L-203 | 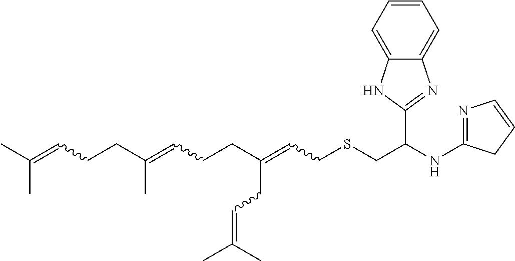 | 518.80 | N-[1-(1H-1,3 - benzodiazol-2-yl)-2- {[7,11-dimethyl- 3-(3-methylbut-2- en-1-yl)dodeca- 2,6,10-trien-1- yl]sulfanyl}ethyl]- 3,4-dihydro-2H- pyrrol-5-amine |
| L-204 | 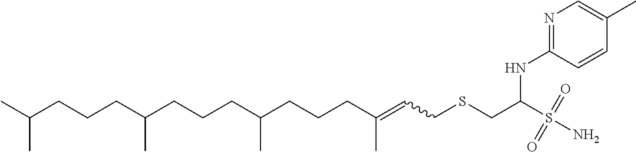 | 525.85 | 5-methyl-N- {1-sulfamoyl-2- [(3,7,11,15- tetramethylhexadec- 2- en-1-yl)sulfanyl] ethyl}pyridin-2- amine |
| L-205 | 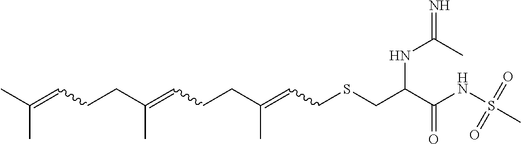 | 443.67 | 2- ethanimidamido- N- methanesulfonyl- 3-[(3,7,11- trimethyldodeca- 2,6,10-trien-1- yl)sulfanyl] propanamide |
| L-206 | 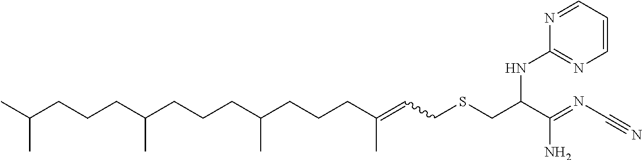 | 500.79 | N′-cyano-2- (pyrimidin-2- ylamino)- 3-[(3,7,11,15- tetramethylhexadec- 2- en-1-yl)sulfanyl] propanimidamide |
| L-207 | 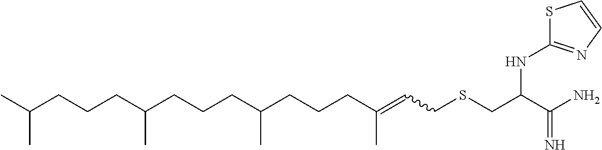 | 480.82 | 3-[(3,7,11,15- tetramethylhexadec- 2- en-1-yl)sulfanyl]-2- (1,3-thiazol-2- ylamino) propanimidamide |
| L-208 | 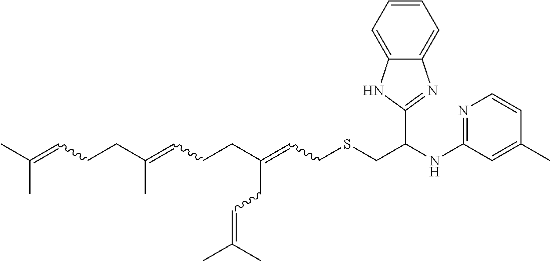 | 542.82 | N-[1-(1H-1,3- benzodiazol-2-yl)-2- {[7,11-dimethyl-3- (3-methylbut-2- en-1-yl)dodeca- 2,6,10-trien-1- yl]sulfanyl}ethyl]- 4-methylpyridin- 2-amine |
| L-209 | 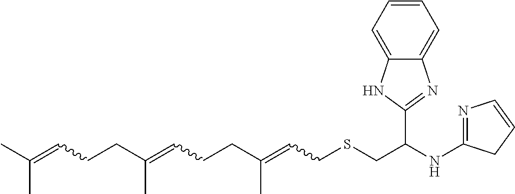 | 464.71 | N-[1-(1H-1,3- benzodiazol-2-yl)-2- [(3,7,11- trimethyldodeca- 2,6,10- trien-1-yl)sulfanyl] ethyl]-3,4- dihydro-2H- pyrrol-5-amine |
| L-210 | 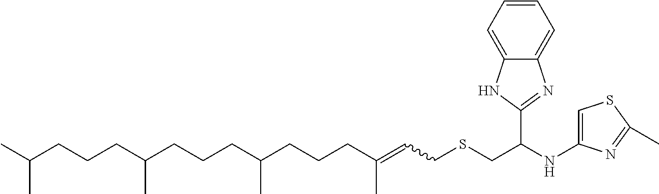 | 568.92 | N-[1-(1H-1,3- benzodiazo1- 2-yl)-2- [(3,7,11,15- tetramethylhexadec- 2- en-1-yl)sulfanyl] ethyl]-2- methyl-1,3- thiazol-4-amine |
| L-211 | 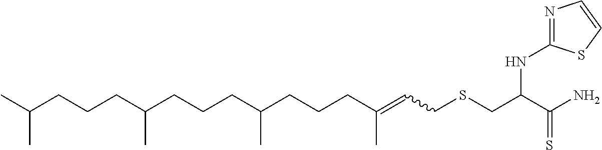 | 497.87 | 3-[(3,7,11,15- tetramethylhexadec- 2- en-1-yl)sulfanyl]- 2-(1,3-thiazol-2- ylamino) propanethioamide |
| L-212 | 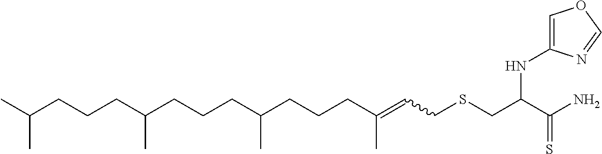 | 481.80 | 2-(1,3-oxazol- 4-ylamino)-3- [(3,7,11,15- tetramethylhexadec- 2- en-1-yl)sulfanyl] propanethioamide |
| L-213 | 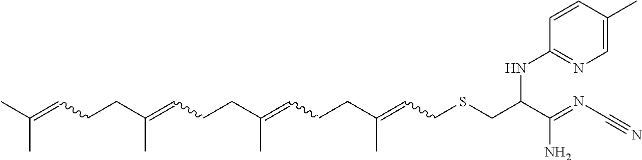 | 507.78 | N′-cyano-2-[(5- methylpyridin-2- yl)amino]-3- {[3,7,11,15- tetramethylhexadeca- 2,6,10,14- tetraen-1- yl]sulfanyl} propanimidamide |
| L-214 | 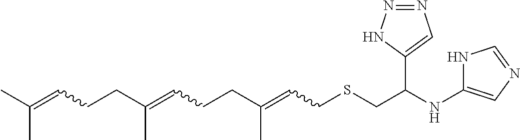 | 414.61 | N-[1-(1H-1,2,3- triazol-5-yl)-2- [(3,7,11- trimethyldodeca- 2,6,10- trien-1-yl) sulfanyl]ethyl]-1H- imidazol-5-amine |
| L-215 | 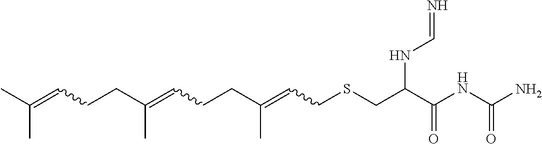 | 394.58 | {2- methanimidamido- 3-[(3,7,11- trimethyldodeca- 2,6,10-trien-1- yl)sulfanyl] propanoyl}urea |
| L-216 | 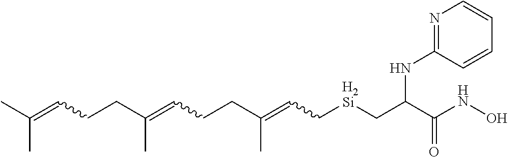 | 464.50 | N-hydroxy-2- (pyridin-2- ylamino)-3- [(3,7,11- trimethyldodeca- 2,6,10- trien-1-yl) selanyl] propanamide |
| L-217 | 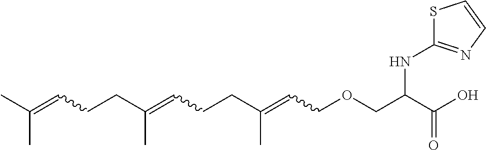 | 392.55 | 2-(1,3-thiazol- 2-ylamino)-3- [(3,7,11- trimethyldodeca- 2,6,10- trien-1-yl)oxy] propanoic acid |
| L-218 | 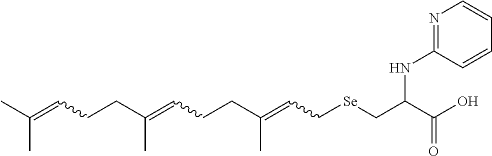 | 449.49 | 2-(pyridin-2- ylamino)-3- [(3,7,11- trimethyldodeca- 2,6,10-trien-1- yl)selanyl] propanoic acid |
| L-219 | 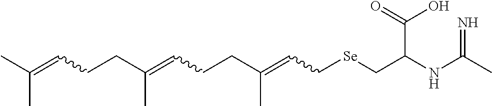 | 413.46 | 2- ethanimidamido- 3-[(3,7,11- trimethyldodeca- 2,6,10-trien-1- yl)selanyl] propanoic acid |
| L-220 | 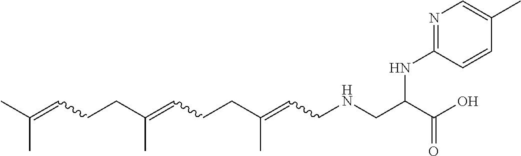 | 399.57 | 2-[(5- methylpyridin-2- yl)amino]-3- [(3,7,11- trimethyldodeca- 2,6,10- trien-1-yl)amino] propanoic acid |

Compounds of the present invention include enantiomers, diastereomers, and double bond isomers of formulae I, Ia and/or Ib.

Compounds described herein (e.g., compounds of I, Ia and/or Ib) may be provided according to the present invention in any of a variety of useful forms, for example as pharmaceutically acceptable salts, as particular crystal forms, etc. In some embodiments, prodrugs of such compounds are provided. Various forms of prodrugs are known in the art, for example as discussed in Bundgaard (ed.), Design of Prodrugs, Elsevier (1985); Widder et al. (ed.), Methods in Enzymology, vol. 4, Academic Press (1985); Kgrogsgaard-Larsen et al. (ed.); “Design and Application of Prodrugs”, Textbook of Drug Design and Development, Chapter 5, 113-191 (1991); Bundgaard et al., Journal of Drug Delivery Reviews, 8:1-38 (1992); Bundgaard et al., J. Pharmaceutical Sciences, 77:285 et seq. (1988); and Higuchi and Stella (eds.), Prodrugs as Novel Drug Delivery Systems, American Chemical Society (1975).

Compounds of the present invention may contain one or more chiral centers and/or double bonds. Unless otherwise stated, the present invention encompasses all isomeric forms (e.g., double bond isomers, enantiomeric, diastereomeric, and geometric and/or conformational) of the compounds and structures are explicitly depicted herein; for example, the R and S configurations for each asymmetric center, Z and E double bond isomers. Therefore, single stereochemical isomers as well as enantiomeric, diastereomeric, geometric and/or conformational mixtures of depicted compounds herein that are within the scope of the invention. Unless otherwise stated, all tautomeric forms of compounds of the present invention are within the scope of the invention, whether as distinct tautomers or a mixture of tautomers.

Unless otherwise indicated, the present invention encompasses racemic forms of compounds depicted herein as well as all enantiomers and stereoisomers, that is, both the stereomerically pure form (e.g., geometrically pure, enantiomerically pure, or diastereomerically pure) and enantiomeric and stereoisomeric mixtures.

A compound may be considered optically active or enantiomerically pure (i.e., substantially the R-form or substantially the S-form) with respect to a chiral center when a compound is about 90% ee (enantiomeric excess) or greater, preferably, equal to or greater than 95% ee with respect to a particular chiral center. A compound may be considered to be in enantiomerically enriched form when a compound has an enantiomeric excess of greater than about 80% ee, preferably greater than about. As used herein, a racemic mixture means about 50% of one enantiomer and about 50% of its corresponding enantiomer relative to all chiral centers in the molecule. Thus, compounds of the present invention may encompass enantiomerically pure, enantiomerically enriched, and racemic mixtures.

Enantiomeric and stereoisomeric mixtures may be resolved into their component enantiomers or stereoisomers by well known methods, such as chiral-phase gas chromatography, chiral-phase high performance liquid chromatography, crystallizing a compound as a chiral salt complex, or crystallizing a compound in a chiral solvent or by enzymatic resolution of a compound, its precursor or its derivative. Enantiomers and stereoisomers may also be obtained from stereomerically or enantiomerically pure intermediates, reagents, and catalysts by well-known asymmetric synthetic methods.

Additionally, unless otherwise stated, the present invention encompasses compounds that differ from those explicitly depicted herein only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention. In certain embodiments, the R¹ group of I, Ia and/or Ib comprises one or more deuterium atoms. In certain embodiments, the R² group of I, Ia and/or Ib comprises one or more deuterium atoms. In certain embodiments, the R³ group of I and/or Ia comprises one or more deuterium atoms. In certain embodiments, the R⁴ group of I and/or Ia comprises one or more deuterium atoms. In certain embodiments, the R¹³ group of Ib comprises one or more deuterium atoms. Mixtures of isomeric forms may be separated and/or purified by techniques as would be known to one skilled in this art, including but not limited to column chromatography.

In certain embodiments, provided compounds modulate a G-protein signaling cascade. In certain embodiments, provided compounds inhibit inflammation. In certain embodiments, activity of provided compounds may be characterized using a variety of in vivo or in vitro assays. For example, ability of provided compounds to inhibit inflammation may be assessed, for example, using assays that assess edema, erythema, and/or inhibition of myeloperoxidase (“MPO”) as described, for example, in Example 21.

In certain embodiments, provided compounds are considered to be inhibitors of inflammation when they show a percent inhibition in an edema assay of at least about 5, 10, 15, 20, 25, 30, 35, 40, 50, 60, 70, 80, 90 or 95%, for example when provided at a dose 0.8 mg/20 μL. In certain embodiments, provided compounds are considered to be inhibitors of inflammation when they show a percent inhibition in an edema assay of at least about 5, 10, 15, 20, 25, 30, 35, 40, 50, 60, 70, 80, 90 or 95%, for example when provided at a dose of 0.2 mg/20 μL.

In certain embodiments, provided compounds are considered to be inhibitors of inflammation when they show a percent inhibition in an erythema assay of at least about 5, 10, 15, 20, 25, 30, 35, 40, 50, 60, 70, 80, 90 or 95%, for example when provided at a dose of 0.8 mg/20 μl. In certain embodiments, provided compounds are considered to be inhibitors of inflammation when they show a percent inhibition in an erythema assay of at least about 5, 10, 15, 20, 25, 30, 35, 40, 50, 60, 70, 80, 90 or 95%, for example when provided at a dose of 0.2 mg/20 μL.

In certain embodiments, provided compounds are considered to be inhibitors of inflammation when they show a percent inhibition in an MPO assay of at least about 5, 10, 15, 20, 25, 30, 35, 40, 50, 60, 70, 80, 90 or 95%, for example when provided at a dose of 0.8 mg/20 μL. In certain embodiments, provided compounds are considered to be inhibitors of inflammation when they show a percent inhibition in an MPO assay of at least about 5, 10, 15, 20, 25, 30, 35, 40, 50, 60, 70, 80, 90 or 95%, for example when provided at a dose of 0.2 mg/20 μL.

### 2. Methods of Syntheses

The present invention provides methods of preparing compounds provided herein. As will be appreciated by one of skill in the art, synthetic methods described herein may be modified without departing from the scope of the present invention. For example, different starting materials and/or different reagents may be used in inventive synthetic methods described herein.

Exemplary methods of syntheses are illustrated in Schemes 1-5 and Example 20. Starting materials useful for preparing compounds and intermediates therefore, are commercially available or may be prepared from commercially available materials using known synthetic methods and reagents.

Protecting groups utilized herein typically denote groups which generally may not be found in final therapeutic compounds but which may intentionally be introduced at some stage of a synthesis in order to protect groups that otherwise might be altered in the course of chemical manipulations. Such protecting groups may be removed or converted to the desired group at a later stage of the synthesis, and compounds bearing such protecting groups thus may be of importance primarily as chemical intermediates (although some derivatives also exhibit biological activity). Accordingly, the precise structure of the protecting group is not critical. Numerous reactions for the formation and removal of such protecting groups are described in a number of standard works including, for example, “Protective Groups in Organic Chemistry”, Plenum Press, London and New York, 1973; Greene, Th. W. “Protective Groups in Organic Synthesis”, Wiley, New York, 1981; “The Peptides”, Vol. I, Schroder and Lubke, Academic Press, London and New York, 1965; “Methoden der organischen Chemie”, Houben-Weyl, 4th Edition, Vol. 15/I, Georg Thieme Verlag, Stuttgart 1974, the disclosures of which are incorporated herein by reference.

Scheme 1 below illustrates a general methodology for the general synthesis of compounds of the formulae I or Ia,

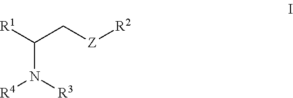

or a pharmaceutically acceptable salt, enantiomer, diastereomer, or double bond isomer thereof, wherein:

R¹ is an optionally substituted heteroaryl group or:

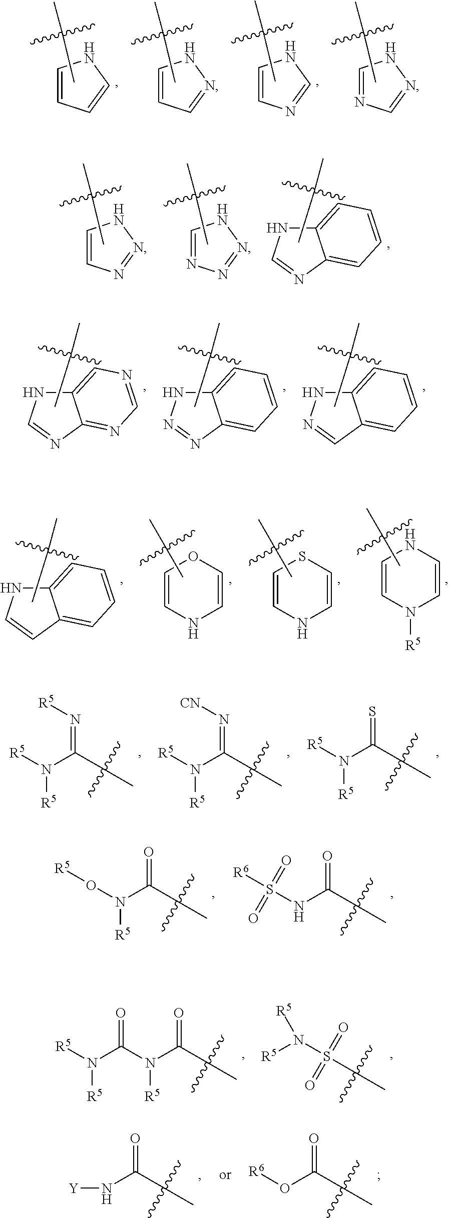

R² is an aliphatic group substituted with one or more R⁷ groups;

R³ is an optionally substituted heteroaryl group,

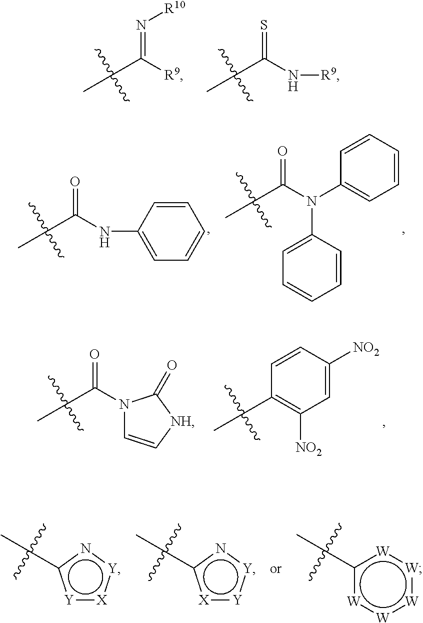

W is independently —C(R¹²)— or N;

R¹² is halo, hydrogen, CF₃, N(R⁵)₂, oxo, alkyl, alkenyl, alkynyl or aryl;

X is —O—, S, —N—, —N(R⁵)—, —C(R¹¹)— or —C(R⁶)—;

Y is independently —C(R¹¹)—, N or —OH;

R¹¹ is hydrogen, F, CH₃, CF₃, OH, —NH₂, —NHNH₂, alkyl, alkenyl, alkynyl or aryl;

R⁴ is H, alkyl, aryl, alkenyl, alkynyl, or a cyclic radical, wherein R⁴ is optionally substituted with one or two R⁷ groups;

R⁵ is independently H, alkyl, aryl, alkenyl, or alkynyl, or —C(═O)O-t-butyl wherein R⁵ is optionally substituted with one or two R⁷ groups;

R⁶ is H, alkyl, aryl, alkenyl, alkynyl, or a cyclic radical, wherein R⁶ is optionally substituted with one or two R⁷ groups;

R⁷ is —NHC(═O)(C₁-C₈)alkyl, —(C₁-C₈)alkyl, —(C₁-C₈)alkenyl, —(C₁-C₈)alkynyl, phenyl, —(C₂-C₅)heteroaryl, —(C₁-C₆)heterocycloalkyl, —(C₃-C₇)cycloalkyl, —O—(C₁-C₈)alkyl, —O—(C₁-C₈)alkenyl, —O—(C₁-C₈)alkynyl, —O-phenyl, —CN, —OH, oxo, halo, —C(═O)OH, —COhalo, —OC(═O)halo, —CF₃, N₃, NO₂, —NH₂, —NH((C₁-C₈)alkyl), —N((C₁-C₈)alkyl)₂, —NH(phenyl), —N(phenyl)₂, —C(═O)NH₂, —C(═O)NH((C₁-C₈)alkyl), —C(═O)N((C₁-C₈)alkyl)₂, —C(═O)NH(phenyl), —C(═O)N(phenyl)₂, —OC(═O)NH₂, —NHOH, —NOH((C₁-C₈)alkyl), —NOH(phenyl), —OC(═O)NH((C₁-C₈)alkyl), —OC(═O)N((C₁-C₈)alkyl)₂, —OC(═O)NH(phenyl), —OC(═O)N(phenyl)₂, —CHO, —CO((C₁-C₈)alkyl), —CO(phenyl), —C(═O)O((C₁-C₈)alkyl), —C(═O)O(phenyl), —OC(═O)((C₁-C₈)alkyl), —OC(═O)(phenyl), —OC(═O)O((C₁-C₈)alkyl), —OC(═O)O(phenyl), —S—(C₁-C₈)alkyl, —S—(C₁-C₈)alkenyl, —S—(C₁-C₈)alkynyl, and —S-phenyl, —NHS(O)₂-phenyl, —NHS(O)₂-alkyl, —NHS(O)₂—(C₁-C₈)alkenyl, —NHS(O)₂—(C₁-C₈)alkynyl, —NHS(O)₂, —SC(O)-phenyl, —SC(O)-alkyl, —SC(O)—(C₁-C₈)alkenyl, —SC(O)—(C₁-C₈ alkynyl), —O—S(═O)₂—(C₁-C₈)alkyl, —O—S(═O)₂—(C₁-C₈)alkenyl, —O—S(═O)₂—(C₁-C₈)alkynyl, —O—S(═O)₂-phenyl, —(CH₂)ₙNH₂, —(CH₂)ₙ—NH((C₁-C₈)alkyl), —(CH₂)ₙN((C₁-C₈)alkyl)₂, —(CH₂)ₙNH(phenyl), or —(CH₂)ₙN(phenyl)₂, wherein n is 1 to 8.

R⁹ is H, alkyl, alkenyl, alkynyl, aryl, —N(R⁵)₂;

R¹⁰ is H, alkyl, alkenyl, alkynyl, aryl, —CN, —S(═O)₂—R⁶ or —C(═O)O-t-butyl; and

Z is —S—, —O—, —Se—, —S(O)—, —SO₂—, or —NH—;

wherein, each of the dashed lines independently represents the presence or absence of a double bond.

In certain embodiments, inventive compounds are prepared as shown in Scheme 1 below.

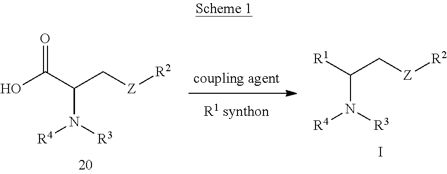

According to one method for the synthesis of compound I, compound 20 can be coupled to an R¹ synthon by using a coupling agent under appropriate conditions. For example, compound 20 can be coupled to the R¹ synthon by adopting the synthetic methods and reagents described in M. P. Cava and M. I. Levinson, Tetrahedron, 1985, 41, 5061-5087; Rachita and Slough, Tetrahedron Letters, 1993, 34, 6821-24; Ishizuka, et al., Synthesis, 2000, 784-88; Johnson and Widlanski, Tetrahedron Letters, 42, 3677-79; Ishizuka, et al., Synthesis, 2000, 784-88; Kumar, et al., JOC, 1996, 4462-65, Koguro, et al., Synthesis, 1998, 910-914; and Racanè, et al., Monatschefte für Chemie, 2006, 137, 1571-1577, which references are hereby incorporated herein by reference.

Suitable coupling agents include, but are not limited to, O-(7-azabenzotriazol-1-yl)-N,N,N′,N′-tetramethyluronium hexafluorophosphate (HATU), N,N′-dicyclohexylcarbodiimide (DCC), N-Ethyl-N-(3-dimethylaminopropyl)carbodiimide (EDC), 1,1′-carbonyldiimidazole (CDI), (benzotriazol-1-yloxy)tripyrrolidinophosphonium hexafluorophosphate (PyBop), N,N′-diisopropylcarbodiimide (DIC)/1-hydroxybenzotriazole (HOBt), N,N,N″,N′-Tetramethyl-O-(1H-benzotriazol-1-yl)uronium hexafluorophosphate (HBTU).

The product I may be purified according to well known methods such as chromatography, and the product analyzed by low resolution mass spectrometry and/or NMR.

When R¹ (see compound I in Scheme 1) is an aromatic heterocyclic group, compound I can be prepared according to the method of Scheme 2.

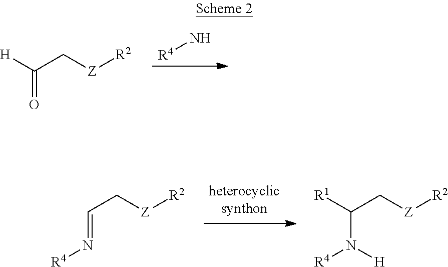

In one embodiment, starting material is reacted with a heterocyclic synthon according to the synthetic methods described in Kim, et al. Bioorganic and Medicinal Chemistry Letters 14 (2004) 4651-4, or Moulin, et al. Synthesis—Stuttgart 17 (2007) 2667-73, which references are hereby incorporated herein by reference.

Alternately, when R⁴ is H, the R³ group can be added directly via the same synthetic route according to the method of Scheme 3:

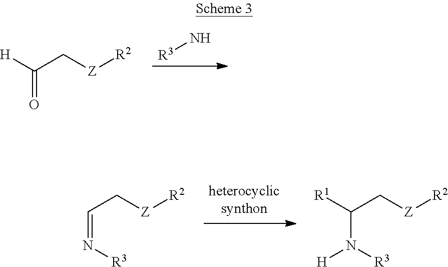

Examples of suitable R¹ groups are shown below. These may be made with synthons such as NaN₃, TMS, etc.

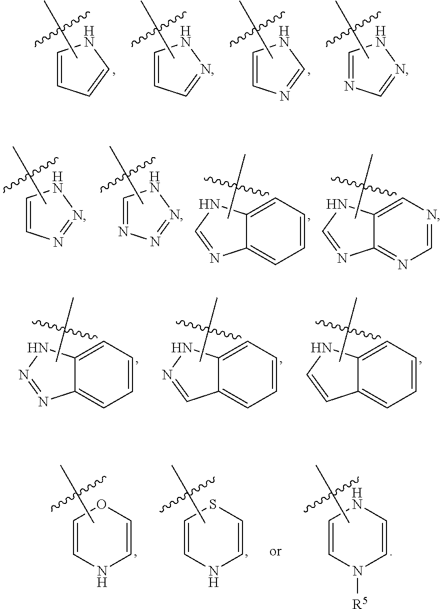

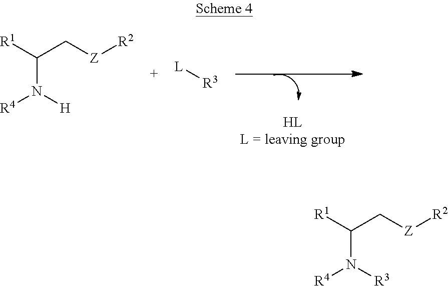

A heterocyclic group, R³, with a reactive leaving group L, such as Br (or another halogen), can be coupled to the amino group under appropriate conditions. One example of this method is described in Ito et al. Biological & Pharmaceutical Bulletin. 2007 30:1838-1843.

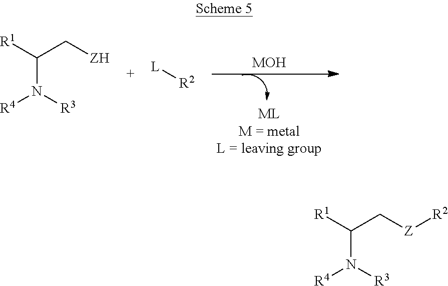

A C₁₀ to C₂₅ R² group can be added in the presence of a base according to the method of Scheme 5 where, for example, M is a metal such Na or K, and L is a suitable leaving group such as Br or Cl. Alternately, the reaction may be carried out in the presence of an organic base, such as triethylamine, where L forms a salt with the base.

For example, farnesol is converted into the corresponding farnesyl bromide by reaction with one half equivalent of PBr₃ in the presence of base, stirred at 0° C. for 1 hour. The same method, or with substitution of PCl₃, can be used to generate any activated lipid starting from an alcohol, for example, from phytol, or geranylgeraniol.

Cysteine hydrochloride, selenocysteine, and derivatives or related compounds containing a thiol or selenol can be reacted with the brominated or chlorinated lipid in, for example, ethanol with K₂CO₃ as base at room temperature with stirring for 3 hrs.

A methodology for the general synthesis of compounds of formula Ib is described herein as Example 20,

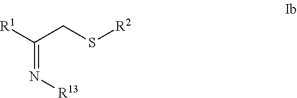

or a pharmaceutically acceptable salt, enantiomer, diastereomer, or double bond isomer thereof, wherein:

R¹ is an optionally substituted heteroaryl group or:

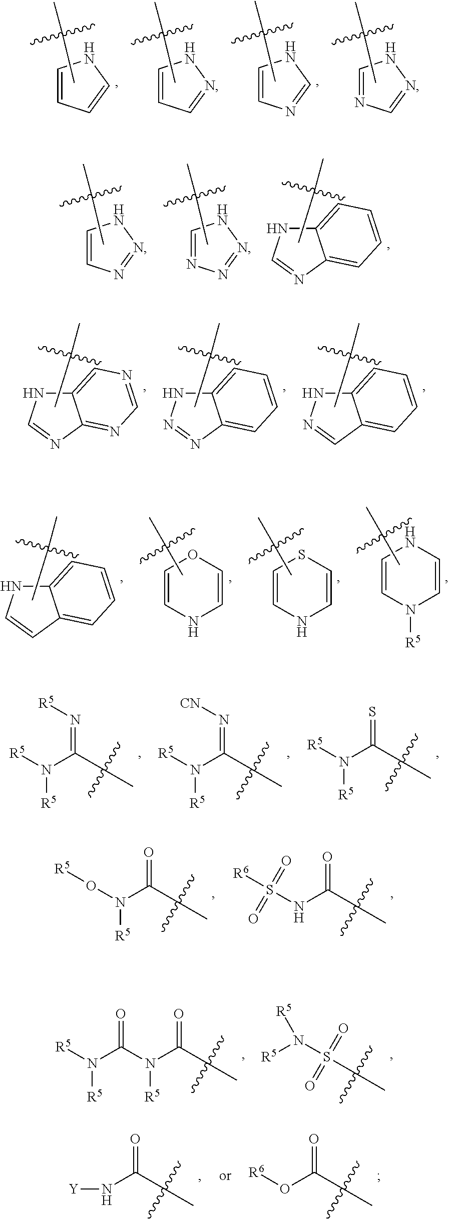

R² is an aliphatic group substituted with one or more R⁷ groups;

R¹³ is independently H,

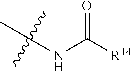

### or —NH—S(O)₂R¹⁴;

R¹⁴ is independently H, (C1-C4)alkyl or aryl;

R⁵ is independently H, alkyl, aryl, alkenyl, or alkynyl, wherein R⁵ is optionally substituted with one or two R⁷ groups;

R⁶ is H, alkyl, aryl, alkenyl, alkynyl, or a cyclic radical, wherein R⁶ is optionally substituted with one or two R⁷ groups;

R⁷ is —NHC(═O)(C₁-C₈)alkyl, —(C₁-C₈)alkyl, —(C₁-C₈)alkenyl, —(C₁-C₈)alkynyl, phenyl, —(C₂-C₅)heteroaryl, —(C₁-C₆)heterocycloalkyl, —(C₃-C₇)cycloalkyl, —O—(C₁-C₈)alkyl, —O—(C₁-C₈)alkenyl, —O—(C₁-C₈)alkynyl, —O-phenyl, —CN, —OH, oxo, halo, —C(═O)OH, —COhalo, —OC(═O)halo, —CF₃, N₃, NO₂, —NH₂, —NH((C₁-C₈)alkyl), —N((C₁-C₈)alkyl)₂, —NH(phenyl), —N(phenyl)₂, —C(═O)NH₂, —C(═O)NH((C₁-C₈)alkyl), —C(═O)N((C₁-C₈)alkyl)₂, —C(═O)NH(phenyl), —C(═O)N(phenyl)₂, —OC(═O)NH₂, —NHOH, —NOH((C₁-C₈)alkyl), —NOH(phenyl), —OC(═O)NH((C₁-C₈)alkyl), —OC(═O)N((C₁-C₈)alkyl)₂, —OC(═O)NH(phenyl), —OC(═O)N(phenyl)₂, —CHO, —CO((C₁-C₈)alkyl), —CO(phenyl), —C(═O)O((C₁-C₈)alkyl), —C(═O)O(phenyl), —OC(═O)((C₁-C₈)alkyl), —OC(═O)(phenyl), —OC(═O)O((C₁-C₈)alkyl), —OC(═O)O(phenyl), —S—(C₁-C₈)alkyl, —S—(C₁-C₈)alkenyl, —S—(C₁-C₈)alkynyl, and —S-phenyl, —NHS(O)₂-phenyl, —NHS(O)₂-alkyl, —NHS(O)₂—(C₁-C₈)alkenyl, —NHS(O)₂—(C₁-C₈)alkynyl, —NHS(O)₂, —SC(O)-phenyl, —SC(O)-alkyl, —SC(O)—(C₁-C₈)alkenyl, —SC(O)—(C₁-C₈ alkynyl), —O—S(═O)₂—(C₁-C₈)alkyl, —O—S(═O)₂—(C₁-C₈)alkenyl, —O—S(═O)₂—(C₁-C₈)alkynyl, —O—S(═O)₂-phenyl, —(CH₂)ₙNH₂, —(CH₂)ₙ—NH((C₁-C₈)alkyl), —(CH₂)ₙN((C₁-C₈)allyl)₂, —(CH₂)ₙNH(phenyl), or —(CH₂)ₙN(phenyl)₂, wherein n is 1 to 8.

In the above-described scheme and/or steps, the R¹, R², R³, R⁴, Z, L, HL, ML, MOH, of the various formulae are as described herein.

### 3. Uses

As described herein, the present invention relates, among other things, to treating or lessening the severity of one or more diseases in which protein inhibitors that modulate the G-protein signaling cascade play a role. Specifically, the present invention relates to methods of treating or lessening the severity of inflammatory diseases or disorders selected from inflammation (acute or chronic), inflammatory diseases or disorders (e.g., asthma, autoimmune diseases, and COPD including emphysema, chronic bronchitis and small airways disease, etc.), inflammatory responses of the immune system, skin diseases (e.g., reducing acute skin irritation for patients suffering from rosacea, atopic dermatitis, seborrheic dermatitis, psoriasis), irritable bowel syndrome (e.g., Chron's disease and ulcerative colitis, etc.), and Parkinson's disease, wherein the method comprises administering to a patient in need thereof a composition of the present invention.

In certain embodiments, provided compounds of the present invention are capable of effectively inhibiting inflammatory responses that are mediated by G-proteins or GPCRs in neutrophils, macrophages and platelets. Thus, provided compounds are inhibitors of edema, erythema and myeloperoxidase and are therefore useful for treating one or more disorders associated with inflammatory diseases or disorders as described herein. In particular, the present invention encompasses the finding that certain compounds having superior in vivo activity than other compounds in the same class. For example, compound A, compound B, compound C, compound D, compound E, compound F, compound G, compound H, compound I, compound J, and/or compound K show edema inhibition, erythema inhibition and MPO (myeloperoxidase) inhibition. Therefore, such compounds are administered to a subject suffering from or susceptible to one or more inflammatory diseases or disorders.

In certain embodiments, treatment of inflammatory diseases or disorders is achieved using compounds without having side effects observed with corticosteroids or NSAIDS.

In certain embodiments, such compounds are administered in vitro. In certain embodiments such compounds are administered in vivo.

Another aspect of the present invention is directed to methods of treating, preventing, or ameliorating inflammation by administering an effective amount of a provided compound.

In some embodiments, one or more inventive compounds, alone or together with one or more other pharmaceutically active agents, is used to whiten skin. In some such embodiments, the compound is applied topically.

In general, the actual quantity of provided compounds of the invention administered to a patient (e.g., in an individual dose or via a dosing regimen) will vary depending on the severity and type of indication, the mode of administration, the particular compound used, the formulation used, and the response desired.

The dosage for treatment is administration, by any of the foregoing means or any other means known in the art, of an amount sufficient to bring about the desired therapeutic effect. Thus, an effective amount includes an amount of a provided compound (or mixture of provided compounds) or pharmaceutical composition of this invention that is sufficient to induce a desired effect, including specifically an anti-inflammation effect.

In general, provided compounds of the present invention are highly active. For example, a provided compound can be administered at about 10 μg/kg to about 50 mg/kg body weight, depending on the specific provided compound selected, the desired therapeutic response, the route of administration, the formulation and other factors known to those of skill in the art.

### 4. Compositions and Formulations

Compounds of the present invention may be formulated into pharmaceutical compositions with at least one compound described herein together with one or more pharmaceutically acceptable carriers, including but not limited to excipients, such as diluents, carriers and the like, and additives, such as stabilizing agents, preservatives, solubilizing agents, buffers and the like, as may be desired.

In certain embodiments, the present invention provides pharmaceutical compositions comprising a compound and a pharmaceutically acceptable carrier. The carrier may be a liquid formulation, and is preferably a buffered, isotonic, aqueous solution. Pharmaceutically acceptable carriers may also be excipients, such as diluents, carriers and the like, and additives, such as stabilizing agents, preservatives, solubilizing agents, buffers and the like, as hereafter described.

Formulation excipients may include but are not limited to polyvinylpyrrolidone, gelatin, hydroxy cellulose, acacia, polyethylene glycol, manniton, sodium chloride and sodium citrate. For injection or other liquid administration formulations, water containing at least one or more buffering constituents is preferred, and stabilizing agents, preservatives and solubilizing agents may also be employed. For solid administration formulations, any of a variety of thickening, filler, bulking and carrier additives may be employed, for example, starches, sugars, fatty acids and the like. For topical administration formulations, any of a variety of creams, ointments, gels, lotions and the like may be employed. For most pharmaceutical formulations, non-active ingredients may constitute the greater part, by weight or volume, of the preparation. For pharmaceutical formulations, it is also contemplated that any of a variety of measured-release, slow-release or time-release formulations and additives may be employed, so that the dosage may. be formulated so as to effect delivery of a compound of the invention over a period of time. For example, gelatin, sodium carboxymethylcellulose and/or other cellulosic excipients may be included to provide time-release or slower-release formulations, especially for administration by subcutaneous and intramuscular injection.

Compounds described herein may be combined as the active ingredient in an admixture with a pharmaceutical carrier according to conventional pharmaceutical compounding techniques. The carrier may take a wide variety of forms depending on the form of preparation desired for administration, for example, oral, topical, parenteral (including intravenous), urethral, vaginal, nasal, dermal, transdermal, pulmonary, deep lung, inhalation, buccal, sublingual, or the like. In preparing compositions of the present invention for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating-agents, lubricants, binders, disintegrating agents and the like in the case of oral solid preparations such as, for example, powders, hard and soft capsules and tablets.

Tablets and capsules may represent an advantageous oral dosage unit form. If desired, a composition with a compound of the invention may be coated by standard aqueous or nonaqueous techniques. The amount of active compound is such that an effective dosage will be obtained. In another embodiment, sublingual pharmaceutical compositions may be employed, such as sheets, wafers, tablets or the like. The active compound may also be administered intranasally as, for example, by liquid drops or spray.

The tablets, pills, capsules, and the like may also contain a binder such as gum tragacanth, acacia, corn starch or gelatin; excipients such as dicalcium phosphate; a disintegrating agent such as corn starch, potato starch or alginic acid; a lubricant such as magnesium stearate; and a sweetening agent such as sucrose, lactose or saccharin. When a dosage unit form is a capsule, it may contain, in addition to materials of the above type, a liquid carrier such as a fatty oil.

Compounds of the present invention may also be administered parenterally. Solutions or suspensions of active peptides may be prepared in water suitably mixed with a surfactant, such as hydroxypropylcellulose. Dispersions may also be prepared, such as dispersions in glycerol, liquid polyethylene glycols and mixtures thereof in oils. These preparations may optionally contain a preservative to prevent the growth of microorganisms. Lyophilized single unit formulations may also be utilized, which are reconstituted, such as with saline, immediately prior to administration.

Pharmaceutical forms suitable for injectable use may include but are not limited to, for example, sterile aqueous solutions or dispersions and sterile powders, such as lyophilized formulations, for the extemporaneous preparation of sterile injectable solutions or dispersions. The form may be sterile and must be fluid to the extent that it may be administered by syringe. The form must be stable under the conditions of manufacture and storage and may be preserved against the contaminating action of microorganisms such as bacteria and fungi. The carrier may be a solvent or dispersion medium containing, for example, water, ethanol, a polyol, for example glycerol, propylene glycol or liquid polyethylene glycol, suitable mixtures thereof, and vegetable oils.

If in an aqueous solution, compounds of the present invention may be appropriately buffered by means of saline, acetate, phosphate, citrate, acetate or other buffering agents, which may be at any physiologically acceptable pH, generally from about pH 4 to about pH 7. A combination of buffering agents may also be employed, such as phosphate buffered saline, a saline and acetate buffer, and the like. In the case of saline, a 0.9% saline solution may be employed. In the case of acetate, phosphate, citrate, acetate and the like, a 50 mM solution may be employed. In addition to buffering agents, a suitable preservative may be employed, to prevent or limit bacteria and other microbial growth. One such preservative that may. be employed is 0.05% benzalkonium chloride.

Compounds of the present invention may be administered in a dried and particulate form. In a preferred embodiment, the particles are between about 0.5 and 6.0 μm, such that the particles have sufficient mass to settle on the lung surface, and not be exhaled, but are small enough that they are not deposited on surfaces of the air passages prior to reaching the lung. Any of a variety of different techniques may be used to make dry powder microparticles, including but not limited to micro-milling, spray drying and a quick freeze aerosol followed by lyophilization. With micro-particles, the compounds of the invention may be deposited to the deep lung, thereby providing quick and efficient absorption into the bloodstream. Further, with such approach penetration enhancers are not required, as is sometimes the case in transdermal, nasal or oral mucosal delivery routes. Any of a variety of inhalers may be employed, including but not limited to propellant-based aerosols, nebulizers, single dose dry powder inhalers and multidose dry powder inhalers. Common devices in current use include but are not limited to metered dose inhalers, which may be used to deliver medications for asthma treatment, chronic obstructive pulmonary disease and the like. Preferred devices include dry powder inhalers, designed to form a cloud or aerosol of fine powder with a particle size that is always less than about 6.0 μm.

Microparticle size, such as mean size distribution, may be controlled by means of the method of making. For micro-milling, the size of the milling head, speed of the rotor, time of processing and the like may control the microparticle size. For spray drying, the nozzle size, flow rate, dryer heat and the like may control the microparticle size. For making by means of quick freeze aerosol followed by lyophilization, the nozzle size, flow rate, concentration of aerosoled solution and the like may control the microparticle size. These parameters and others may be employed to control the microparticle size.

Compounds of the present invention may be therapeutically administered by means of an injection, typically a deep intramuscular injection, for example, in the gluteal or deltoid muscle, of a time release injectable formulation. In one embodiment, a compound may be formulated with a PEG, for example, poly(ethylene glycol) 3350, and optionally one or more additional excipients and preservatives, including but not limited to excipients such as salts, polysorbate 80, sodium hydroxide or hydrochloric acid to adjust pH, and the like. In another embodiment, a compound is formulated with a poly(ortho ester), which may be an auto-catalyzed poly(ortho ester) with any of a variable percentage of lactic acid in the polymeric backbone, and optionally one or more additional excipients. In one embodiment poly (D,L-lactide-co-glycolide) polymer (PLGA polymer) is employed, preferably a PLGA polymer with a hydrophilic end group, such as PLGA RG502H from Boehringer Ingelheim, Inc. (Ingelheim, Germany).

Formulations may be made, for example, by combining a compound in a suitable solvent, such as methanol, with a solution of PLGA in methylene chloride, and adding thereto a continuous phase solution of polyvinyl alcohol under suitable, mixing conditions in a reactor. In general, any of a number of injectable and biodegradable polymers, which may also be adhesive to polymers, may be employed in a time release injectable formulation. The teachings of U.S. Pat. Nos. 4,938,763, 6,432,438, and 6,673,767, and the biodegradable polymers and methods of formulation disclosed therein, are incorporated herein by reference. The formulation may be such that an injection is required on a weekly, monthly or other periodic basis, depending on the concentration and amount. of compound, the biodegradation rate of the polymer, and other factors known to those of skill in the art.

### 5. Routes of Administration

Compounds and/or compositions of the present invention are suitable to be administered, for example, orally, topically, nasally, parenterally (including intravenous), urethrally, vaginally, dermally, transdermally, buccally, sublingually by pulmonary administration, deep lung administration or by inhalation, or the like.

If administered by injection, the injection may be intravenous, subcutaneous, intramuscular, intraperitoneal or other means known in the art. Compounds may be formulated by any means known in the art, including but not limited to formulation as tablets, capsules, caplets, suspensions, powders, lyophilized preparations, suppositories, ocular drops, skin patches, oral soluble formulations, sprays, aerosols and the like, and may be mixed and formulated with buffers, binders, excipients, stabilizers, anti-oxidants and other agents known in the art. In general, any route of administration by which the compounds are introduced across an epidermal layer of cells may be employed. Administration means may thus include administration through mucous membranes, buccal administration, oral administration, dermal administration, inhalation administration, pulmonary administration, nasal administration, urethral administration, vaginal administration, and the like.

A compound of the invention may be administered by means of a time-release injectable formulation, such compound may be in a formulation with a PEG, poly(ortho ester) or PLGA polymer. In another aspect, a compound may be administered by means of an automated delivery device providing subcutaneous delivery, either continuous or intermittent. Any of the foregoing methods and formulations may be applicable for treatment of chronic conditions or syndromes, including but not limited to chronic congestive heart failure and particularly chronic decompensated congestive heart failure.

Compounds of the present invention may also be administered by transdermal administration, including by means of the delivery system, including the apparatus, but not limited to the methods as disclosed in U.S. Patent Application Publication 2006/0034903. Similarly, the hydrogel formulations and solid state formulations disclosed therein may be adapted for use with the compounds.

### 6. Dosage

### Therapeutically Effective Amount

The actual quantity of compounds administered to a patient will vary depending on the severity and type of indication, the mode of administration, the particular compound used, the formulation used, and the response desired.

The dosage for treatment is administration, by any of the foregoing means or any other means known in the art, of an amount sufficient to bring about the desired therapeutic effect. Thus, a therapeutically effective amount may be an amount of a compound or pharmaceutical composition that is sufficient to induce a desired effect, including but not limited to an anti-inflammation effect. Those of ordinary skill in the art will appreciate that a therapeutically effective amount may be administered by means of a single dose or multiple doses, and that compositions provided herein may contain a unit dose of a therapeutically effective amount.

In general, provided compounds are highly active. For example, a compound may be administered at about 10 μg/kg to about 50 mg/kg body weight, depending on the specific compound selected, the desired therapeutic response, the route of administration, the formulation and other factors known to those of skill in the art.

### 7. Combination Therapy

It is contemplated that a provided compound can be used in combination with other drugs or therapeutic agents.

In some embodiments, compounds as described herein are administered in combination with one or more other agents intended to treat the same condition, or disease (or symptoms thereof). As used herein, additional therapeutic agents that are normally administered to treat a particular disease, or condition, are known as “appropriate for the disease, or condition, being treated.”

For example, in some embodiments, compounds of the present invention, or a pharmaceutically acceptable composition thereof, are administered in combination with other anti-inflammatory agents to treat inflammatory diseases and/or disorders. Examples of known anti-inflammatory agents include, but are not limited to, dexamethasone, indomethacin and clobetasol.

In some embodiments, compounds of the present invention are administered in combination with one or more other pharmaceutically active agents intended to treat a different disease, disorder, or condition. For example, in some embodiments, it may be desirable to administer an inventive compound in order to reduce inflammation while concurrently administering a different pharmaceutically active agent in order to achieve a different biological result.

To give but one example, it is known that transdermal administration of pharmaceutically active agents often causes skin irritation at the site of delivery. Indeed, it is not uncommon that a skin irritating agent (e.g., SDS) be administered prior to or concurrent with application of a transdermal device such as, for example, a transdermal patch, in order to facilitate the delivery. Applicant has found that addition or co-administration of a compound as described herein in combination with transdermal administration of another pharmaceutically active agent can reduce inflammation and/or irritation associated with the transdermal administration of the other pharmaceutically active agent.

Indeed, Applicant has found that addition or co-administration of compounds of the present invention (i.e., polyisoprenyl protein inhibitor compounds) can similarly reduce inflammation and/or irritation associated with the transdermal administration of another pharmaceutically active agent. Such polyisoprenyl protein inhibitor compounds useful in accordance with this aspect of the present invention not only include related compounds as described in co-pending U.S. provisional patent application Ser. No. 12/326,746, which is incorporated herein by reference, but also includes compounds of the present invention. Amounts of compound used in the device may vary, depending on many factors including the size of the device and its release characteristics, the amount of the pharmaceutical active agent and the estimated duration of action of the device. Broadly, amounts of the compound range from about 0.1% to about 10% w/v.

It is also known that single or chronic injections of a pharmaceutically active agent may sometimes result in inflammation, whether due to the identity of the pharmaceutically active agent (i.e., as an irritant) or to the mode of delivery. The present invention contemplates co-administration of one or more compounds of the present invention, and/or one or more other compounds that are structurally related to AFC (i.e., polyisoprenyl protein inhibitor compounds), in order to reduce inflammation associated with single or chronic injection of a pharmaceutically active agent. In these cases, the polyisoprenyl-protein inhibitor may be administered topically, formulated in an appropriate pharmaceutically acceptable carrier. See, e.g., U.S. Patent Application Publication Number 2005/0277694.

Exemplary pharmaceutically active agents whose delivery, whether transdermally or by injection, may cause skin irritation include levadopa, pro-drug forms of levadopa, insulin, estradiol, estrogen, progesterone, progestins, progestogen, testosterone, nicotine, nitroglycerin, cholinesterase inhibitors, stimulants, antidepressants, and analgesics.

To give another example, application of certain agents such as, for example, hair relaxants, which commonly are or contain basic agents (e.g., NaOH), can cause skin irritation (e.g., irritation and/or inflammation of the scalp). According to the present invention, one or more compounds of the present invention, and/or one or more other polyisoprenyl protein inhibitor compounds, can be administered together with such a hair relaxant (or other agent) to reduce skin irritation and/or inflammation.

Although the invention has been described in detail with particular reference to these preferred embodiments, other embodiments can achieve the same results. Variations and modifications of the present invention will be obvious to those skilled in the art and it is intended to cover all such modifications and equivalents. The entire disclosures of all references, applications, patents, and publications cited above and/or in the attachments, and of the corresponding application(s), are hereby incorporated by reference.

### EXAMPLES

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all classes, subclasses and species of each of these compounds, disclosed herein.

The following general experimental procedures were used in the Examples described below. Proton Nuclear Magnetic Resonance (¹HNMR) spectroscopy was recorded on a Bruker 500 MHz spectrometer, dimethyl sulfoxide (DMSO-d6), methanol (CD₃OD) or chloroform (CDCl₃) was used as ¹H-NMR solvent. The residual proton absorption of the deuterated solvent was used as the internal standard. All ¹H-NMR chemical shift are reported as δ values in the parts per million (ppm). The splitting pattern abbreviations are as follows: s, singlet; d, doublet; t, triplet; q, quartet; br, broad; m, multiplet; dd, doublet of doublet; dt, doublet of triplets. The HPLC analysis was done using a phenomenex luna C₁₈(2)50×4.6 mm column. The mobile phase is 60% water, 40% acetonitrile containing 0.05% trifluoroacetic acid at 2 ml per minute flow rate for the first 2.5 minutes, followed by a gradient to 100% acetonitrile containing 0.05% TFA over 10 minutes. The eluent is observed at 214 nm.

### Example 1

### Synthesis of Compounds

### Wherein R³=Pyridine Derivative

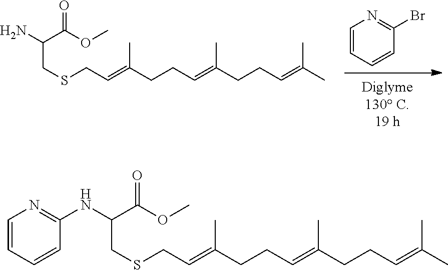

2-bromopyridine is added to a solution of farnesylcysteine methyl ester in diethyleneglycol dimethyl ether, and heated to 130° C. under nitrogen for 19 hours. The reaction mixture is diluted with aqueous ammonia and the product is extracted into chloroform. The combined organic phases are dried over anhydrous sodium sulfate, and the solvent is removed in vacuo to afford the crude product, which can be purified by reverse phase HPLC. The protecting methyl group on the —COOH may be hydrolyzed under basic conditions before or after purification. Details of this method are described in Ito et al. Biological & Pharmaceutical Bulletin. 2007 30: pp. 1838-1843.

### Example 2

### Synthesis of Compounds

### Wherein R¹⁰=CN

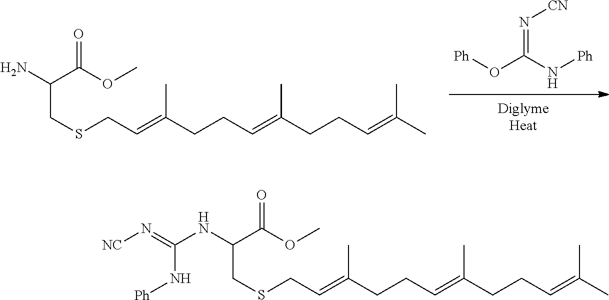

Useful synthetic methods are disclosed in U.S. Pat. No. 4,514,400 (Campbell et al.), which are incorporated herein by reference. To a solution of farnesylcysteine methyl ester in diethyleneglycol dimethyl ether, O-phenyl-N-cyano-N′-phenylisourea is added and heated under nitrogen to yield the product. The protecting methyl group on the —COOH may be hydrolyzed under basic conditions before or after purification.

### Example 3

### Synthesis of Tetrazole Derivatives

A nitrile intermediate is synthesized by reacting 1,4-dithiane-2,5-diol with an activated lipid (2 equivalents), such as farnesyl bromide, under suitable conditions, e.g., THF, with a base, e.g., K₂CO₃, stirring at room temperature. The intermediate acetaldehyde is converted to the nitrile by Strecker synthesis, for example by using KCN with NH₄OH in water, to add the desired R³ group and to protect the free amine. Alternately, the free amine may be temporarily protected, such as with a -Boc or acetate group, which must be hydrolyzed before addition of the desired R³ and/or R⁴ groups on the free amine.

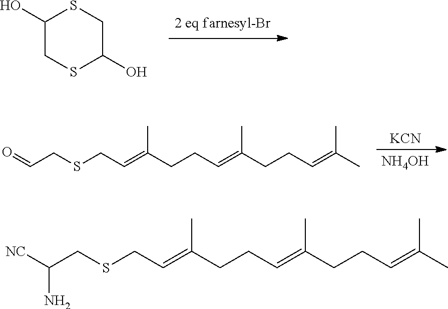

Heterocyclic derivatives at the R¹ position are synthesized from a nitrile intermediate by heating with NaN₃ or a similar synthon in DMF and NH₄Cl as solvent, such as by following the methods described in Kumar et al. J. Org. Chem. 1996, 61:4462-4465, and Koguro, et al., Synthesis, 1998, 1998:910-914.

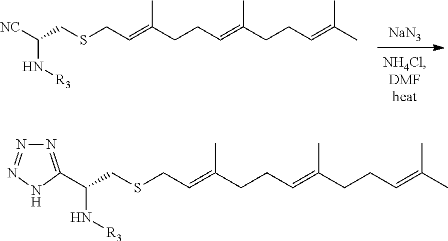

### Example 4

### Synthesis of Substituted Amidine Derivatives

Amidine derivatives at the R¹ position are synthesized from a nitrile intermediate (see example 3) by reacting first with an alcohol (such as methanol) and an acid and next, by heating and refluxing with a snython having the formula NH₂R′ in EtOH as a solvent, such as by following the methods described in Racanè, et al., Monatschefte für Chemie, 2006, 137:1571-1577.

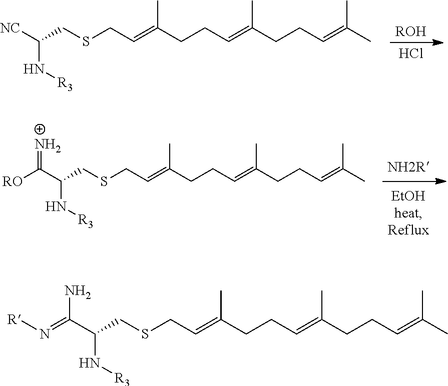

### Example 5

### Synthesis of Bis-Substituted Amidine Derivatives

Amidine derivatives at the R¹ position are synthesized from a nitrile intermediate (see example 3) by reacting first with an alcohol (such as methanol) and an acid and next by heating and refluxing with a synthon having the formula NHR′R″ in EtOH as a solvent, such as by following the methods described in Racanè, et al., Monatschefte für Chemie, 2006, 137:1571-1577.

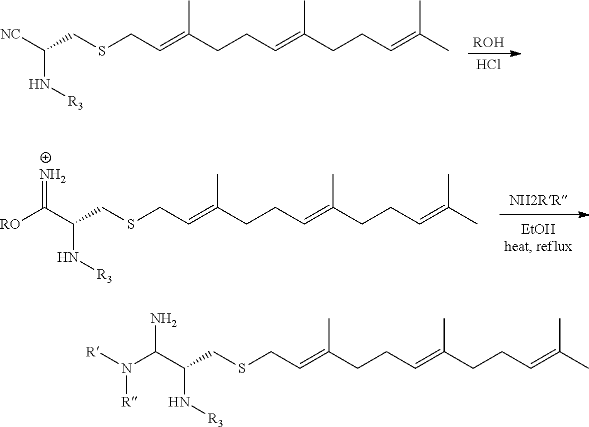

### Example 6

### Synthesis of Thioamide Derivatives

Combine an amino-amide derivative of cysteine with a Lawesson's reagent in THF. The subsequent step modifies the free amine by reacting with a synthon as needed to yield the desired R³. Methods of the synthesis are described in M. P. Cava and M. I. Levinson, Tetrahedron, 1985, 41:5061-5087 and Z. Kaleta, et al, Org. Lett. 2006, 8:1625-1628.

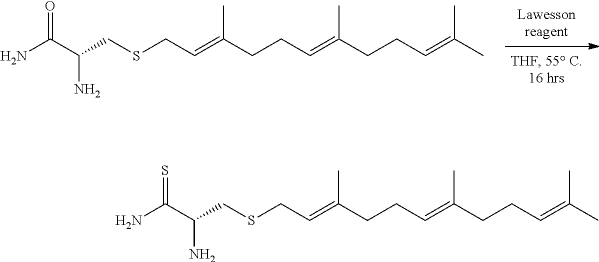

### Example 7

### Synthesis of Sulfonamide Derivatives

Combine a boc-protected chloro-derivative of a lipidated cysteine, or a chloro-deravitive protected by an alternate group (such as acetyl) on the amine, and a substituted sulfonamide in DMF. The boc group is hydrolyzed with an acid and the free amine reacted with a synthon as needed to yield the desired R³. Methods of synthesis are described in Johansson et al. Journal of Combinatorial Chemistry 2000, 2:496-507; Rachita and Slough, Tetrahedron Letters, 1993, 34:6821-24; and Ishizuka, et al., Synthesis, 2000, 2000:784-88.

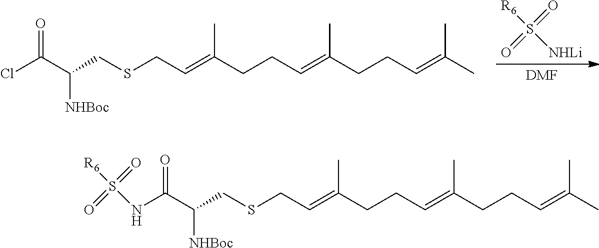

### Example 8

### Synthesis of Imidazole Derivatives

According to the methods of Blessing et al. Proc. Natl. Acad. Sci. USA. 1998. 95:1427-31, a guanidino intermediate is synthesized by reacting a free amine compound in acetonitrile with a boc-protected 1H-pyrazole-1-carboxamidine. For example, farnesyl cysteine with a —COOH protecting group is reacted with a methyl ester, as shown below:

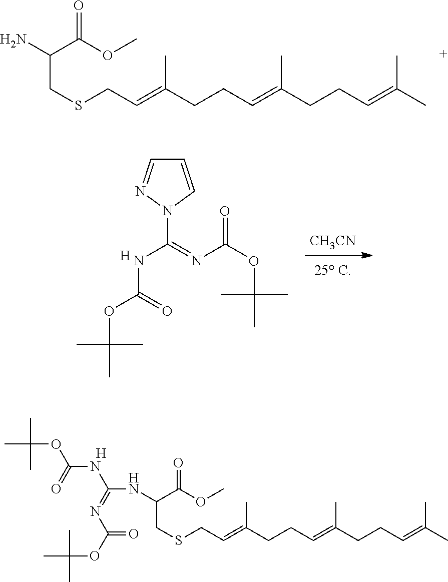

The boc protecting groups is removed with TFA, to yield guanidine-farnesyl cysteine. To a solution of guanidino-farnesyl cysteine, in anhydrous DMF, 0.4-1.0 equivalents of bromoacetaldehyde is added and stirred at room temperature under nitrogen for 4 days. The crude product is evaporated to dryness and washed with water to afford the crude product, which can be purified by column chromatography. The methyl protecting group is hydrolyzed under basic conditions before, or after, purification. The product is further modified following Scheme 1, above, to yield a product with an alternate R¹.

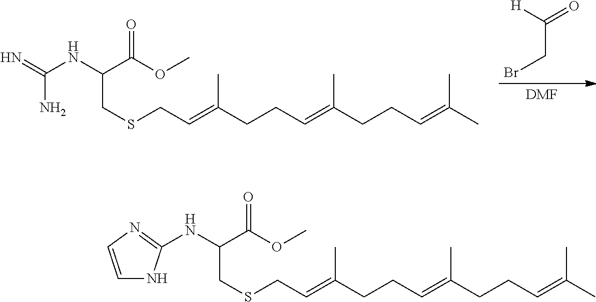

### Example 9

### Synthesis of Compound A

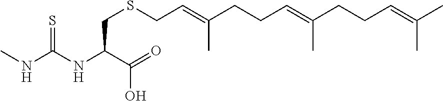

In 24 mL vial, S-trans, trans-farnesyl-L-cysteine (325 mg, 1.0 mmole) and N,N-diisopropyl ethyl amine (650 mg, 5.0 mmole) were dissolved in methylene chloride (10 mL). Ethyl thioisocyanate (73 mg, 1.0 mmole) was added to the reaction mixture. The reaction mixture was stirred at room temperature overnight. Methylene chloride was removed by rotary evaporation. The crude material was dissolved in ethyl acetate (100 mL) and washed with saturated NH₄Cl solution (50 mL). The ethyl acetate solution was dried on Na₂SO₄ and concentrated. The crude reaction mixture was purified by preparative HPLC (80 mg, 20% yield): ¹H-NMR (500 MHz, CDCl₃) δ 1.60 (s, 6H), 1.68 (s, 6H), 1.95-2.10 (m, 8H), 2.65 (d, J=9.2 14.2 Hz, 1H), 3.07 (dd, J=3.5, 14.2 Hz, 1H), 3.08-3.27 (m, 2H), 3.25 (s, 3H), 4.23 (m, 1H), 5.09 (m, 2H), 5.31 (m, 1H); ¹³C-NMR (125 MHz, CDCl₃) δ 16.1, 16.3, 17.7, 25.7, 26.3, 26.4, 26.7, 27.6, 30.1, 30.2, 32.6, 32.9, 39.5, 39.6, 39.7, 56.6, 59.3, 119.3, 119.4, 123.6, 123.7, 124.3, 131.4, 135.4, 135.6, 140.5, 140.7, 172.7, 184.3; ES-MS: mass calcd for Chemical Formula: C₂₀H₃₄N₂O₂S₂ 398.2 (M+). Found (M+Na) m/z 421.3.

### Example 10

### Synthesis of Compound B

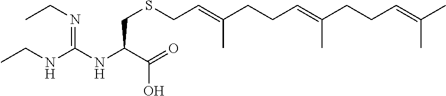

In a 4 mL vial, S-trans, trans-farnesyl-L-cysteine (325 mg, 1.0 mmole) was dissolved in diisopropyl ethyl amine (10 mL). Ethyl isocyanate (142 mg, 2.0 mmole) was added to the reaction mixture. The reaction mixture was stirred at room temperature overnight. The reaction mixture was washed with water (10 mL×2). The remained crude material was purified by preparative HPLC (60 mg, 15% yield): ¹H-NMR (500 MHz, CDCl₃) δ 1.12 (t, J=7.25 Hz, 3H), 1.15 (t, J=7.25 Hz, 3H), 1.59 (S, 3H), 1.60 (S, 3H), 1.67 (S, 3H), 1.68 (S, 3H), 1.95-2.12 (m, 8H), 2.77 (dd, J=7.9, 13.9 Hz), 2.87 (dd, J=5.7, 13.9 Hz), 3.17-3.32 (m, 6H), 4.44 (q, J=7.6, 1H), 5.09 (t, J=7.0 Hz, 2H), 5.15 (t, J=7.6 Hz, 1H); ¹³C-NMR (125 MHz, CDCl₃) δ 14.6, 15.4, 16.0, 16.2, 17.7, 25.7, 26.5, 26.7, 30.1, 34.4, 34.6, 35.2, 39.6, 39.7, 53.4, 113.9, 119.8, 123.7, 123.8, 124.3, 127.8, 131.3, 135.3, 139.8, 157.7, 171.7; ES-MS: mass calcd for Chemical Formula: C₂₃H₄₁N₃O₂S 423.3 (M+). Found (M+Na) m/z 446.3.

### Example 11

### Synthesis of Compound C

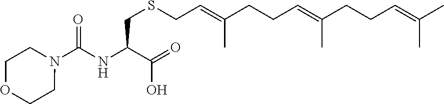

In 100 mL round bottom flask, S-trans, trans-farnesyl-L-cysteine (325 mg, 1.0 mmole) and potassium carbonate (1.3 g, 10 mmole) were dissolved in THF (50 mL). 4-morpholine carbonyl chloride (160 mg, 1.2 mmole) was added. The reaction mixture was stirred at room temperature for 47 hours. THF was removed by rotary evaporation. The crude material was washed with water (10 mL) and 1N HCl solution (10 mL). The remaining crude material was purified by preparative HPLC (120 mg, 27% yield): ¹H-NMR (500 MHz, CDCl₃) δ 1.60 (bs, 6H), 1.66 (s, 3H), 1.67 (s, 3H), 1.95-2.12 (m, 8H), 2.94-3.02 (m, 2H), 3.10-3.20 (m, 2H), 3.40-3.44 (m, 4H), 3.71 (t, J=4.8 Hz, 4H), 4.55 (dd, J=6.3, 11.6 Hz, 1H), 5.09 (t, J=6.9 Hz, 2H), 5.19 (t, J=7.6 Hz, 1H); ¹³C-NMR (125 MHz, CDCl₃) δ 16.0, 16.2, 17.7, 25.7, 26.4, 26.7, 29.9, 33.0, 39.6, 39.7, 44.0, 53.0, 66.3, 119.4, 123.7, 124.3, 131.3, 135.4, 140.2, 157.7, 174.1; ES-MS: mass calcd for Chemical Formula: C₂₃H₃₈N₂O₄S 438.3 (M+). Found (M+Na) m/z 461.4.

### Example 12

### Synthesis of Compound D

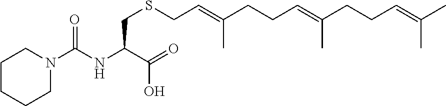

In 24 mL vial, S-trans, trans-farnesyl-L-cysteine (325 mg, 1.0 mmole) and potassium carbonate (1.3 g, 10 mmole) were dissolved in THF (5 mL). 1-piperidine carbonyl chloride (148 mg, 1.0 mmole) was added. The reaction mixture was stirred at room temperature overnight. THF was removed by rotary evaporation. The crude material was dissolved in ethyl acetate (25 mL) and washed with 1N HCl solution (10 mL). The remaining crude material was purified by preparative HPLC (167 mg, 38% yield): ¹H-NMR (500 MHz, CD₃OD) δ 1.50-1.59 (m, 4H), 1.62 (bs, 6H), 1.63-1.67 (m, 2H), 1.69 (s, 3H), 1.71 (s, 3H), 1.98-2.17 (m, 8H), 2.81 (dd, J=8.9, 13.9 Hz, 1H), 3.01 (dd, J=5.0, 13.7 Hz, 1H), 3.15 (dd, J=7.3, 13.3 Hz, 1H), 3.28 (dd, J=8.2, 13.3 Hz, 1H), 3.40-3.43 (m, 4H), 4.55 (dd, J=6.4, 11.5 Hz, 1H), 5.10 (t, J=6.7 Hz, 2H), 5.21 (t, J=7.6 Hz, 1H); ¹³C-NMR (125 MHz, CD₃OD) δ 16.0, 16.2, 17.7, 24.4, 25.7, 27.8, 28.3, 28.4, 30.2, 33.7, 40.8, 40.9, 46.1, 55.0, 121.7, 125.2, 125.5, 132.1, 136.3, 140.4, 159.5, 175.4; ES-MS: mass calcd for Chemical Formula: C₂₄H₄₀N₂O₃S 436.3 (M+). Found (M+Na) m/z 459.3.

### Example 13

### Synthesis of Compound E

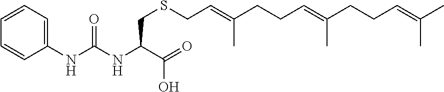

In 24 mL vial, S-trans, trans-farnesyl-L-cysteine (325 mg, 1.0 mmole) and phenyl isocyanate (119 mg, 1.0 mmole) were dissolved in pyridine (5 mL). The reaction mixture was stirred at room temperature overnight. The crude material was dissolved in ethyl acetate (25 mL) and washed with water (2×50 mL). The ethyl acetate solution was dried on Na₂SO₄ and concentrated. The crude reaction mixture was purified by preparative HPLC (220 mg, 50% yield): ¹H-NMR (500 MHz, CDCl₃) δ 1.58 (s, 3H), 1.59 (s, 3H), 1.60 (s, 3H), 1.63 (s, 3H), 1.96-2.07 (m, 8H), 2.92 (dd, J=6.3, 13.9 Hz, 1H), 3.00 (dd, J=4.4, 9.2 Hz, 1H), 3.12-3.21 (m, 2H), 4.66 (bd, J=5.0 Hz, 1H), 5.07 (bd, J=5.34 Hz, 2H), 5.15 (t, J=7.3 Hz, 1H), 7.10-7.35 (m, 5H); ¹³C-NMR. (125 MHz, CDCl₃) δ 16.0, 16.1, 17.7, 25.7, 26.4, 26.7, 30.0, 32.9, 39.6, 39.7, 52.9, 119.4, 121.9, 123.7, 124.3, 124.7, 129.4, 131.4, 135.4, 137.4, 140.2, 156.4, 164.5, 174.5; ES-MS: mass calcd for Chemical Formula: C₂₅H₃₆N₂O₃S 444.2 (M+). Found (M+Na) m/z 467.3.

### Example 14

### Synthesis of Compound F

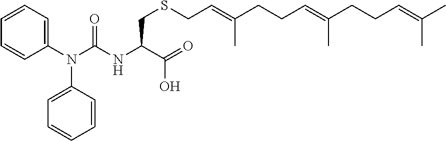

In 24 mL vial, S-trans, trans-farnesyl-L-cysteine (325 mg, 1.0 mmole), diphenyl carbonyl chloride (232 mg, 1.0 mmole), and potassium carbonate (1.3 mg, 10.0 mmole) were dissolved in THF (5 mL). The reaction mixture was stirred at room temperature overnight. Reaction solvent was removed by rotary evaporation. The crude material was dissolved in ethyl acetate (25 mL) and washed with water (2×50 mL). The ethyl acetate solution was dried on Na₂SO₄ and concentrated. The crude reaction mixture was purified by preparative HPLC (140 mg, 27% yield): ¹H-NMR (500 MHz, CD₃OD) δ 1.62 (s, 3H), 1.63 (s, 3H), 1.68 (bs, 6H), 1.98-2.17 (m, 8H), 2.87 (dd, J=6.9, 8.9 Hz, 1H), 2.99 (dd, J=4.8, 14.2 Hz, 1H), 3.12 (dd, J=7.6, 13.2 Hz, 1H), 3.19 (dd, J=8.2, 13.3 Hz, 1H), 4.57 (dd, J=4.4, 6.9 Hz, 1H), 5.10-5.19 (m, 3H), 7.26-7.43 (m, 10H); ¹³C-NMR (125 MHz, CD₃OD) δ 15.3, 15.5, 17.0, 25.1, 26.6, 27.0, 29.6, 33.0, 39.9, 40.1, 53.6, 119.4, 120.7, 124.3, 124.7, 126.9, 129.8, 131.3, 139.8, 143.2, 157.3, 173.6; ES-MS: mass calcd for Chemical Formula: C₃₁H₄₀N₂O₃S 520.3 (M+). Found (M+Na) m/z 543.3.

### Example 15

### Synthesis of Compound G

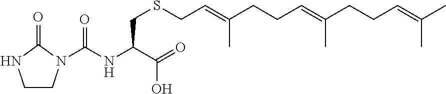

In 24 mL vial, S-trans, trans-farnesyl-L-cysteine (325 mg, 1.0 mmole), imidazolidine carbonyl chloride (150 mg, 1.0 mmole), and potassium carbonate (1.3 g, 10.0 mmole) were dissolved in THF (5 mL). The reaction mixture was stirred at room temperature overnight. Reaction solvent was removed by rotary evaporation. The crude material was dissolved in ethyl acetate (25 mL) and washed with water (2×50 mL). The ethyl acetate solution was dried on Na₂SO₄ and concentrated. The crude reaction mixture was purified by preparative HPLC (162 mg, 37% yield): ¹H-NMR (500 MHz, CD₃OD) δ 1.47 (s, 6H), 1.54 (s, 3H), 1.55 (s, 3H), 1.98-2.17 (m, 8H), 2.77 (dd, J=6.3, 14.2 Hz, 1H), 2.84 (dd, J=4.8, 13.9 Hz, 1H), 3.03 (dd, J=7.6, 13.2 Hz, 1H), 3.11 (dd, J=8.2, 13.3 Hz, 1H), 3.33 (t, J=8.5 Hz, 2H), 3.75 (dt, J=4.8, 8.5 Hz, 2H), 4.49 (t, J=4.8 Hz, 1H), 4.96-4.99 (m, 2H), 5.09 (t, J=7.8 Hz, 1H); ¹³C-NMR (125 MHz, CD₃OD) δ 16.2, 16.3, 17.8, 26.0, 27.4, 27.8, 30.7, 34.0, 37.7, 40.8, 40.9, 43.2, 54.1, 121.7, 125.1, 125.5, 132.1, 136.3, 140.6, 155.3, 160.2, 173.9; ES-MS: mass calcd for Chemical Formula: C₂₂H₃₅N₃O₄S 437.2 (M+). Found (M+Na) m/z 460.3.

### Example 16

### Synthesis of Compound H

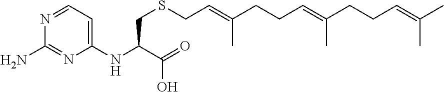

In 20 mL vial, S-trans, trans-farnesyl-L-cysteine (325 mg, 1.0 mmole) was dissolved in isopropanol (10 mL) and diisopropyl ethyl amine (1 mL) followed by addition of 2-amino-4-chloropyrimidine (142 mg, 1.1 mmol). The reaction mixture was heated overnight then reaction mixture was concentrated and purified on preparative HPLC to yield Compound H in 64% yield (215 mg). ¹H NMR (500 MHz, CD₃OD) δ 1.58 (br s, 6H), 1.62 (s, 3H), 1.69 (s, 3H), 1.91-2.17 (m, 8H), 2.89 (dd, J=7.9, 13.9 Hz, 2H), 3.11 (br d, J=13.9 Hz, 2H), 3.20-3.27 (m, 2H), 5.08-5.11 (br s, 2H), 5.22 (t, J=7.9 Hz, 1H), 7.52 (d, J=7.4 Hz, 1H), 8.49 (d, J=7.4 Hz, 1H); ¹³C NMR (125 MHz, CD₃OD) δ 16.2, 17.7, 24.4, 26.0, 27.5, 27.7, 28.7, 29.01, 30.9, 35.3, 40.7, 40.9, 103.4, 122.2, 123.8, 124.2, 124.5, 132.3, 136.5, 139.6, 163.8, 180.4; ES-MS: calcd. for C₂₂H₃₄N₄O₂S: 418.2 m/z (M+). Found: 441.3 m/z (M+Na).

### Example 17

### Synthesis of Compound I

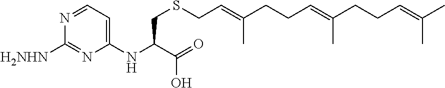

In 20 mL vial, S-trans, trans-farnesyl-L-cysteine (325 mg, 1.0 mmole) was dissolved in isopropanol (10 mL) and diisopropyl ethyl amine (1 mL) followed by addition of 2,4-dichloropyrimidine (153 mg, 1.1 mmol). The reaction mixture was heated overnight then excess of hydrazine (1 mL, 50% aqueous) was added followed by additional heating for 24 hrs. Then reaction mixture was concentrated and purified on preparative HPLC to yield Compound I in 37% yield (160 mg). ¹H NMR (500 MHz CD₃OD) δ 1.47 (br s, 3H), 1.55-1.61 (m, 6H), 1.69 (s, 3H), 1.89-2.17 (m, 8H), 2.92-3.14 (m, 4H), 4.69 (br s, 1H), 5.08-5.12 (br s, 2H), 5.23 (br s, 1H), 6.02 (br s, 1H), 7.64-7.78 (br s, 1H); ¹³C NMR (125 MHz, CD₃OD) δ 16.1, 16.2, 17.7, 25.8, 26.4, 26.5, 26.7, 30.3, 37.6, 39.7, 41.8, 46.3, 101.4, 120.0, 123.8, 124.3, 131.4, 135.3, 139.3, 154.0, 172.4; ES-MS: calcd. for C₂₂H₃₅N₅O₂S: 433.3 m/z (M+). Found: 387.3 m/z (M−CO₂H).

### Example 18

### Synthesis of Compound J

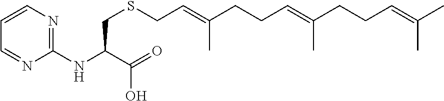

In 20 mL vial, S-trans, trans-farnesyl-L-cysteine (325 mg, 1.0 mmole) was dissolved in isopropanol (10 mL) and diisopropyl ethyl amine (1 mL) followed by addition of 2-chloropyrimidine (126 mg, 1.1 mmol). The reaction mixture was heated overnight then reaction mixture was concentrated and purified on preparative HPLC to yield Compound J in 44% yield (182 mg). ¹H NMR (500 MHz, CDCl₃) δ 1.59 (br s, 6H), 1.62 (s, 3H), 1.71 (s, 3H), 1.93-2.15 (m, 8H), 2.82-3.04 (m, 2H), 3.82 (d, J=5.5 Hz, 2H), 5.04-5.11 (m, 3H), 5.42 (t, J=7.3 Hz, 1H), 6.92 (t, J=4.1 Hz, 1H), 8.49 (d, J=4.1 Hz, 2H); ¹³C NMR (125 MHz, CDCl₃) δ 16.0, 16.2, 17.7, 26.4, 26.5, 28.7, 29.01, 31.2, 39.7, 39.8, 117.8, 119.2, 123.8, 124.5, 131.3, 135.5, 140.6, 157.8, 172.7; ES-MS: calcd. for C₂₂H₃₃N₃O₂S: 403.3 m/z (M+). Found: 426.3 m/z (M+Na).

### Example 19

### Synthesis of Compound K

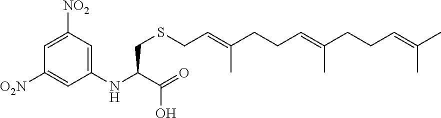

In 24 mL vial, S-trans, trans-farnesyl-L-cysteine (325 mg, 1.0 mmole) and N,N-diisopropyl ethyl amine (650 mg, 5.0 mmole) were dissolved in methylene chloride (5 mL). 2,4-dinitrofuolo benzene (186 mg, 1.0 mmole) was added to the reaction mixture. The reaction mixture was stirred at room temperature overnight. Methylene chloride was removed by rotary evaporation. The crude material was dissolved in ethyl acetate (100 mL) and washed with 10% HCl solution (50 mL). The ethyl acetate solution was dried on Na₂SO₄ and concentrated. The crude reaction mixture was purified by preparative HPLC (280 mg, 57% yield): ¹H-NMR (500 MHz, CDCl₃) δ 1.58 (s, 3H), 1.59 (s, 3H), 1.64 (s, 3H), 1.67 (s, 3H), 1.94-2.07 (m, 8H), 3.14 (d, J=5.0 Hz, 2H), 3.24 (d, J=7.6 Hz, 2H), 4.57 (bd, J=6.6 Hz, 1H), 5.07 (bs, 2H), 5.21 (t, J=7.3 Hz, 1H), 6.84 (d, J=9.4 Hz, 1H), 8.30 (dd, J=2.2, 9.4 Hz, 1H), 9.17 (d, J=2.2 Hz, 1H); ¹³C-NMR (125 MHz, CDCl₃) δ 16.0, 16.2, 17.7, 25.7, 26.3, 6.7, 30.3, 32.9, 39.6, 39.7, 53.4, 55.6, 114.0, 119.1, 123.5, 124.2, 124.3, 130.4, 131.4, 131.5, 135.6, 137.2, 140.9, 146.9, 173.9; ES-MS: mass calcd for Chemical Formula: C₂₄H₃₃N₃O₆S 491.2 (M+). Found (M+Na) m/z 514.3.

### Example 20

### Synthesis of Compounds of Formula Ib

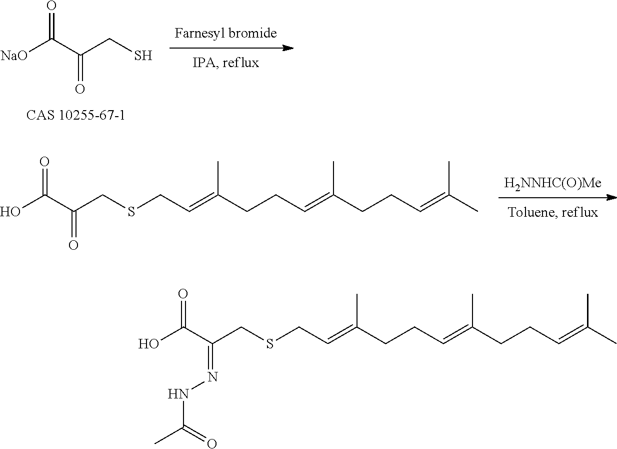

Mercaptopyruvic acid sodium salt is charged to three necked round-bottomed flask equipped with mechanical stirrer, using a pressure equalizing dropping funnel and a thermometer. The flask is placed in a heating mantle and is charged with Na₂CO₃, followed by isopropanol. The stirred suspension is heated to 80° C. and then farnesyl bromide is added drop wise through a dropping funnel over a period of 4-16 hours (the internal temperature of the reaction mixture is maintained from about 78 to about 82° C.). The product is worked up and dried as described in co-pending U.S. application Ser. No. 12/358,712, filed Jan. 23, 2009, the entire disclosure of which is incorporated herein by reference.

The resulting S-farnesyl-mercaptopyruvic acid is dissolved in a toluene three necked round-bottomed flask equipped with a mechanical stirrer, a Dean-Stark trap and a thermometer. The corresponding substituted hydrazine is added in excess and the reaction mixture is heated to a gentle reflux. Addition of an acidic catalyst (e.g. PTSA) is optional. The calculated amount of water is collected in the trap, the reaction is cooled and is worked up to isolate the desired compound of formula Ib. Further purification may also include recrystallization from a suitable organic solvent (e.g. ethanol).

Other examples of the compound of formula I, Ia, and/or Ib include, but are not limited to, those compounds depicted in Table 2 above.

### Example 21

Table 3 below depicts % inhibition determined from an edema assay, an erythema assay, and myeloperoxidase (“MPO”) assay for compound A, compound B, compound C, compound D, compound E, compound F, compound G, compound H, compound I, compound J, and/or compound K.

TABLE 3

| Com- | | | | | |
|---|---|---|---|---|---|
| pound | | | | | |
| Num- | | % Inhibition | | | |
| ber | Structure | Edema | MPO | Erythema | Dose |
| A | 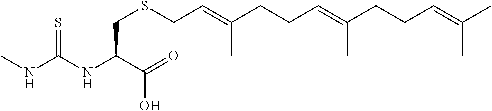 | 8.55 ± 3.7 — | 8.8 ± 6.29 — | (-)3.73 ± 7.7 — | 0.2 mg/ 20 μL — |
| B | 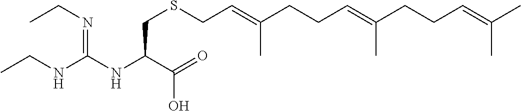 | (-)1.28 ± 4.83 — | (-)26.9 ± 15.32 — | 0.51 ± 10.81 — | 0.2 mg/ 20 μL — |
| C | 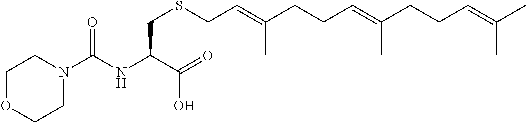 | — 34.09 ± 18.03 | — 61.77 ± 20.44 | — — | — 0.8 mg/ 20 μL |
| D | 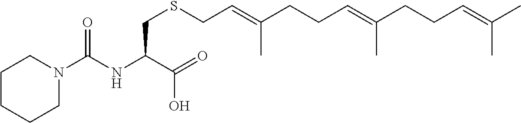 | — 32.62 ± 11.06 | — 83.69 ± 3.71 | — — | — 0.8 mg/ 20 μL |
| E | 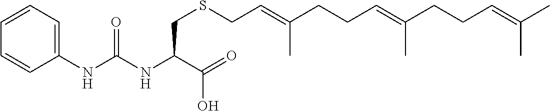 | — 18.22 ± 13.35 | — 75.4 ± 7.20 | — — | — 0.8 mg/ 20 μL |
| F | 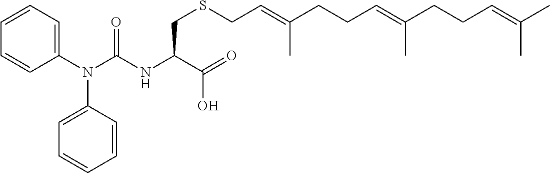 | — 4.66 ± 0.36 | — 79.55 ± 3.32 | — — | — 0.8 mg/ 20 μL |
| G | 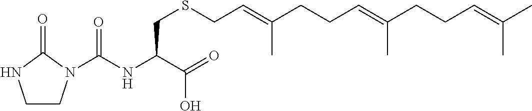 | — 13.39 ± 15.87 | — 57.96 ± 22.86 | — — | — 0.8 mg/ 20 μL |
| H | 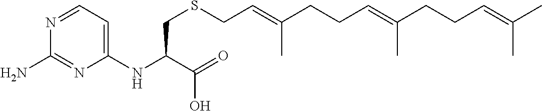 | 39.72 ± 1.67 50.13 ± 14.12 | 63.51 ± 7.03 88.95 ± 3.12 | — — | 0.2 mg/ 20 μL 0.8 mg/ 20 μL |
| I | 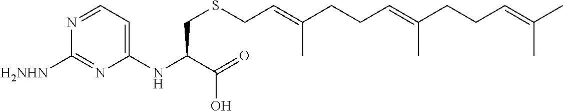 | 24.67 ± 16.47 — | 23.15 ± 6.79 — | (-)28.81 ± 50.44 — | 0.2 mg/ 20 μL — |
| J | 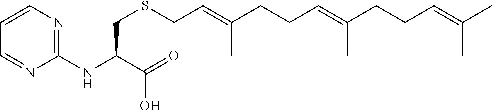 | 1.89 ± 7.75 — | (-)2.13 ± 9.72 — | — — | 0.2 mg/ 20 μL — |
| K | 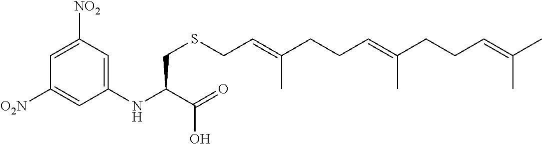 | (-)5.44 ± 6.43 — | 10.37 ± 8.65 — | 45.91 ± 5.31 — | 0.2 mg/ 20 μL — |

### Biological Examples

Described below are in vivo assays used to measure the biological activity of provided compounds, including the anti-inflammatory properties of the compounds, as measured by edema inhibition, erythema inhibition and MPO inhibition.

### Example 22

### Mouse Model of Inflammation-Edema, Erythema and MPO Background

The mouse ear model of contact irritation has been established as an appropriate model to determine whether topically applied anti-inflammatories inhibit the development of acute, chemically induced dermal irritation [see Van Arman, C. G. et al., Anti-inflammatory Drugs, Clin. Pharmacol. Ther. 16, 900-4 (1974); Young et al., Tachyphylaxis in 12-Otetradecanoylphorbolacetate-and Arachidonic Acid-Induced Ear Edema; J. Invest. Dermatol. 80:48-52, (1983); Tramposch et al., In Vivo Models of Inflammation, (Morgan D W, Marshall L A eds), Birkha\ddot{}user Verlag: Basel, pp 179-204, 1999; and Gordon et al., Topical N-Acetyl-S-Farnesyl-L-Cysteine Inhibits Mouse Skin Inflammation, and Unlike Dexamethasone, Its Effects Are Restricted to the Application Site, J. Invest. Dermatol., 128(3):643-54, 2008 March)]. Moreover, the mouse ear model has been used by various groups to identify and compare members of differing classes of anti-inflammatory agents with multiple mechanisms of action (reviewed in Tramposch et al., 1999, supra). The commonly used end points of inflammation are edema (Young et al., 1983, supra), (assayed by increase in ear thickness), neutrophil infiltration (which is measured by assaying for the neutrophil marker myeloperoxidase (“MPO”) (see Bradley et al., Cellular and Extracellular Myeloperoxidase in Pyogenic Inflammation, Blood, 60(3):618-22; 1982) and erythema (skin redness). Using this model, we investigated the in vivo anti-inflammatory activity of S-isoprenyl and S-farnesyl cysteine compounds to identify which structures possess physical or chemical properties critical for inhibiting innate inflammation in the skin.

### (a) Protocol—Edema Inhibition

The protocol for inducing in vivo acute contact inflammation on the ears of live mice has been described elsewhere (reviewed in Tramposch, 1999, supra). In brief, mice were sedated and their ears were treated with 1.2 μg/20 uL TPA (i.e., tetradecanoylphorbol-13-acetate). After 5 minutes, we dosed these TPA-treated ears with a single 8 μg/20 uL dose, a 2 ug/20 uL dose, or both doses, of the S-isoprenyl and S-farnesyl compounds. After 24 hours, the mice were sacrificed and edema was measured by taking micrometer readings of each ear. The percent inhibition of edema was determined by taking the average ear thickness of compound-treated ears and dividing it by the average thickness of 12 ears that only received TPA and subtracting that value from 100%. These values were corrected for the thickness of normal, non TPA-treated mouse ears of littermate controls. Results demonstrating percent inhibition of edema for representative compounds of the present invention are depicted in Table 3. ED₅₀ values were calculated as described in Gordon et al., “Topical N-acetyl-S-farnesyl-L-cysteine Inhibits Mouse Skin Inflammation, and Unlike Dexamethasone, its Effects Are Restricted to the Application Site”, J. Invest. Derm., Vol. 128 pp. 643-654 (2008).

### (b) Protocol-Erythema Inhibition

Another well documented biomarker of skin inflammation is skin redness, termed erythema, which is caused by capillary congestion and dilation in response to various chemical and environmental insults (see Denig, N. I. et al., Irritant Contact Dermatitis. Clues to Causes, Clinical Characteristics, and Control, Postgrad Med., May (1998); 103(5):199-200, 207-8, 212-3). The protocol for measuring erythema inhibition by S-isoprenyl and S-farnesyl cysteine compounds was developed in-house by utilizing the CR-400 chroma meter from Konica Minolta (http://www.konicaminolta.com/instruments/products/color/colorimeters/cr400-410/index.html). This instrument was used to measure the Aa* redness value from 6 mm biopsy punches taken 24 hours post TPA/compound treatment as described in the edema inhibition section above. The percent inhibition of erythema was determined by taking the average Δa* redness value of compound-treated ears and dividing it by the average Δa* value of 12 ears that only received TPA and subtracting that value from 100%. These values were corrected for the Δa* value of non TPA-treated mouse ears of littermate controls. Results demonstrating percent inhibition of erythema for representative compounds of the present invention are depicted in Table 3. Gordon et al., “Topical N-acetyl-S-farnesyl-L-cysteine Inhibits Mouse Skin Inflammation, and Unlike Dexamethasone, its Effects Are Restricted to the Application Site”, J. Invest. Derm., Vol. 128 pp. 643-654 (2008).

### (c) Protocol-MPO Inhibition

To assay for inhibition of dermal neutrophil infiltration by S-isoprenyl and S-farnesyl cysteine compounds, a standard method was used (see Bradley et al., 1982, supra; Young et al., 1983, supra; De Young et al, “Edema and Cell Infiltration in the Phorbol Ester-treated Mouse Ear are Temporally Separate and can be Differentially Modulated by Pharmacologic Agents”, Agents Actions, 26(3-4): 335-41 (March 1989); and Rao et al. (1993) Comparative Evaluation of Arachidonic Acid (AA)-and Tetradecanoylphorbol Acetate (TPA)-Induced Dermal Inflammation, Inflammation 17:723-41). Briefly, we homogenized 6 mm biopsy punches taken from both compound-treated ears as well as TPA-treated and non-treated control groups. We quantitated the levels of MPO by a colorimetric reaction that was measured spectrophotometrically. The percent inhibition of neutrophil infiltration by each S-isoprenyl and S-farnesyl cysteine compound was determined by comparing the average MPO levels in the presence and absence of these compounds. The calculation for percent inhibition of MPO was determined similar to that as described for calculating the percent edema inhibition, see the Edema Inhibition protocol, supra. Results demonstrating percent inhibition of MPO for representative compounds of the present invention are depicted in Table 3. Gordon et al., “Topical N-acetyl-S-farnesyl-L-cysteine Inhibits Mouse Skin Inflammation, and Unlike Dexamethasone, its Effects Are Restricted to the Application Site”, J. Invest. Derm., Vol. 128 pp. 643-654 (2008).

### EQUIVALENTS

Those skilled in the art will recognize, or be able to ascertain using no more than routine experimentation, that while the invention herein has been described with reference to particular embodiments, it is to be understood that these embodiments are merely illustrative of the principles and applications of the present invention and other embodiments may achieve the same results. It is therefore to be understood that numerous modifications may be made to the illustrative embodiments and that other arrangements may be devised without departing from the spirit and scope of the present invention as defined by the appended claims. The preceding examples may be repeated with similar success by substituting the generically or specifically described reactants and/or operating conditions used.

In the claims articles such as “a,” “an,” and “the” may mean one or more than one unless indicated to the contrary or otherwise evident from the context. Claims or descriptions that include “or” between one or more members of a group are considered satisfied if one, more than one, or all group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim. For example, any claim that is dependent on another claim can be modified to include one or more limitations found in any other claim that is dependent on the same base claim.

Where elements are presented as lists, e.g., in Markush group format, it is to be understood that each subgroup of the elements is also disclosed, and any element(s) can be removed from the group. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not been specifically set forth in haec verba herein. It is noted that the term “comprising” is intended to be open and permits the inclusion of additional elements or steps.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any targeting moiety, any disease, disorder, and/or condition, any linking agent, any method of administration, any therapeutic application, etc.) can be excluded from any one or more claims, for any reason, whether or not related to the existence of prior art.

Publications discussed above and throughout the text are provided solely for their disclosure prior to the filing date of the present application. Nothing herein is to be construed as an admission that the inventors are not entitled to antedate such disclosure by virtue of prior disclosure.

1. A compound of formula I: 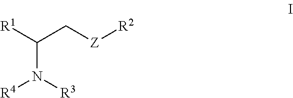
or a pharmaceutically acceptable salt, enantiomer, diastereomer, or double bond isomer thereof, wherein:
R¹ is an optionally substituted heteroaryl group or: 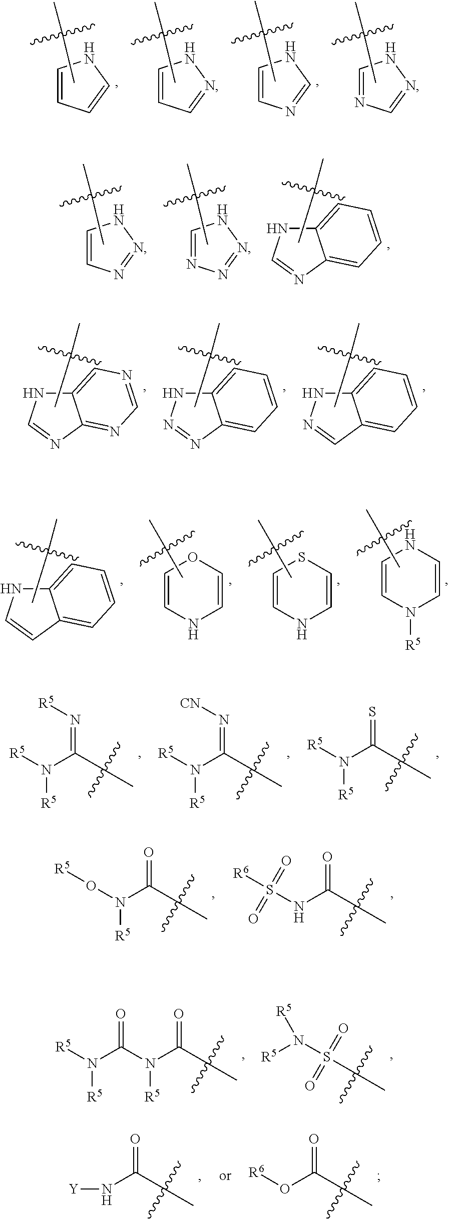
R² is an aliphatic group substituted with one or more R⁷ groups;
R³ is an optionally substituted heteroaryl group, 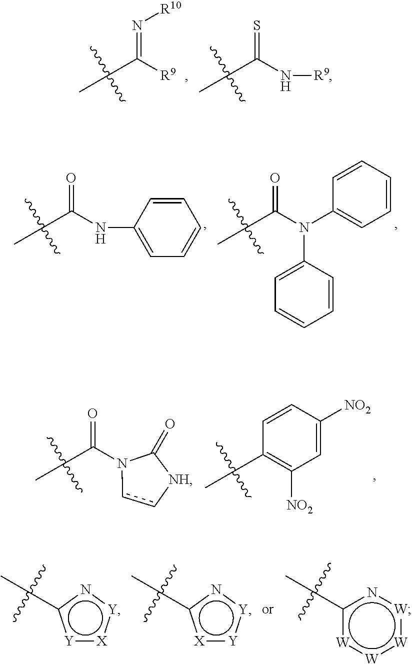
W is independently —C(R¹²)— or N;
R¹² is halo, hydrogen, CF₃, N(R⁵)₂, oxo, alkyl, alkenyl, alkynyl or aryl;
X is —O—, S, —N—, —N(R⁵)—, —C(R¹¹)— or —C(R⁶)—;
Y is independently —C(R¹¹)—, N or —OH;
R¹¹ is hydrogen, F, CH₃, CF₃, OH, —NH₂, —NHNH₂, alkyl, alkenyl, alkynyl or aryl;
R⁴ is H, alkyl, aryl, alkenyl, alkynyl, or a cyclic radical, wherein R⁴ is optionally substituted with one or two R⁷ groups;
R⁵ is independently H, alkyl, aryl, alkenyl, or alkynyl, or —C(═O)O-t-butyl wherein R⁵ is optionally substituted with one or two R⁷ groups;
R⁶ is H, alkyl, aryl, alkenyl, alkynyl, or a cyclic radical, wherein R⁶ is optionally substituted with one or two R⁷ groups;
R⁷ is —NHC(═O)(C₁-C₈)alkyl, —(C₁-C₈)alkenyl, —(C₁-C₈)alkynyl, phenyl, —(C₂-C₅)heteroaryl, —(C₁-C₆)heterocycloalkyl, —(C₃-C₇)cycloalkyl, —O—(C₁-C₈)alkyl, —O—(C₁-C₈)alkenyl, —O—(C₁-C₈)alkynyl, —O-phenyl, —CN, —OH, oxo, halo, —C(═O)OH, —COhalo, —OC(═O)halo, —CF₃, N₃, NO₂, —NH₂, —NH((C₁-C₈)alkyl), —N((C₁-C₈)alkyl)₂, —NH(phenyl), —N(phenyl)₂, —C(═O)NH₂, —C(═O)NH((C₁-C₈)alkyl), —C(═O)N((C₁-C₈)alkyl)₂, —C(═O)NH(phenyl), —C(═O)N(phenyl)₂, —OC(═O)NH₂, —NHOH, —NOH((C₁-C₈)alkyl), —NOH(phenyl), —OC(═O)NH((C₁-C₈)alkyl), —OC(═O)N((C₁-C₈)alkyl)₂, —OC(═O)NH(phenyl), —OC(═O)N(phenyl)₂, —CHO, —CO((C₁-C₈)alkyl), —CO(phenyl), —C(═O)O((C₁-C₈)alkyl), —C(═O)O(phenyl), —OC(═O)((C₁-C₈)alkyl), —OC(═O)(phenyl), —OC(═O)O((C₁-C₈)alkyl), —OC(═O)O(phenyl), —S—(C₁-C₈)alkyl, —S—(C₁-C₈)alkenyl, —S—(C₁-C₈)alkynyl, and —S-phenyl, —NHS(O)₂-phenyl, —NHS(O)₂-alkyl, —NHS(O)₂(C₁-C₈)alkenyl, —NHS(O)₂(C₁-C₈)alkynyl, —NHS(O)₂, —SC(O)-phenyl, —SC(O)-alkyl, —SC(O)—(C₁-C₈)alkenyl, —SC(O)—(C₁-C₈ alkynyl), —O—S(═O)₂—(C₁-C₈)alkyl, —O—S(═O)₂—(C₁-C₈)alkenyl, —O—S(═O)₂—(C₁-C₈)alkynyl, —O—S(═O)₂-phenyl, —(CH₂)ₙNH₂, —(CH₂)ₙ—NH((C₁-C₈)alkyl), —(CH₂)ₙN((C₁-C₈)alkyl)₂, —(CH₂)ₙNH(phenyl), or —(CH₂)ₙN(phenyl)₂, wherein n is 1 to 8.
R⁹ is H, alkyl, alkenyl, alkynyl, aryl, —N(R⁵)₂;
R¹⁰ is H, alkyl, alkenyl, alkynyl, aryl, —CN, —S(═O)₂—R⁶ or —C(═O)O-t-butyl; and
Z is —S—, —O—, —Se—, —S(O)—, —SO₂—, or —NH—;
wherein, each of the dashed lines independently represents the presence or absence of a double bond.

2. The compound according to claim 1, wherein R² is selected from

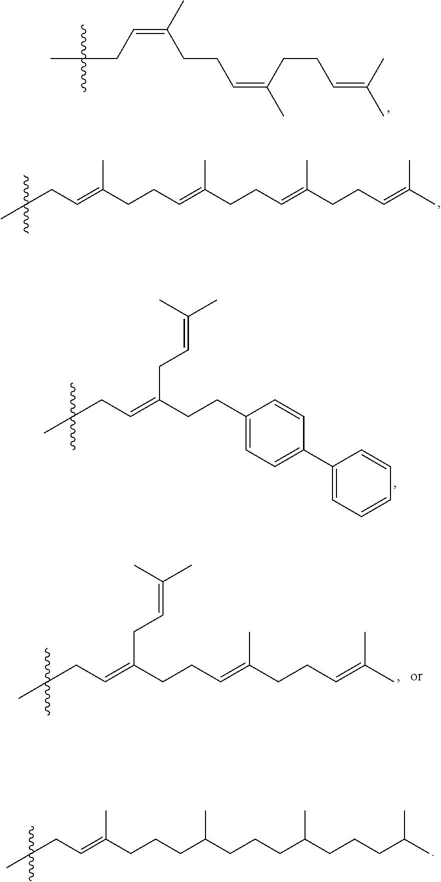

3. The compound according to claim 1, wherein R³ is selected from:

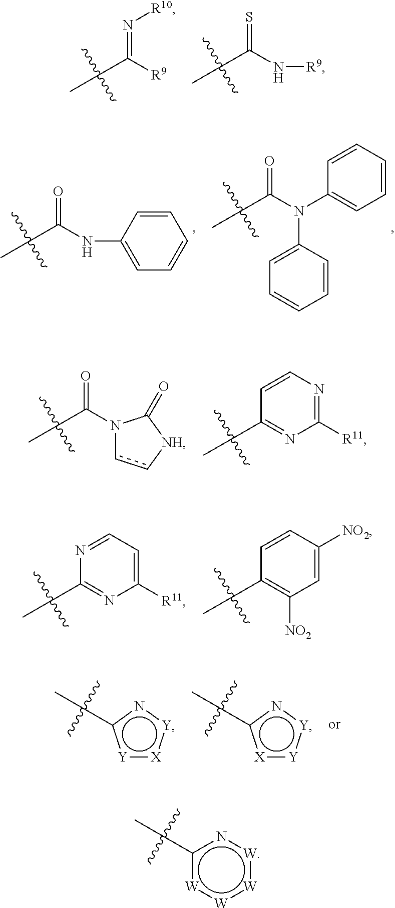

4. The compound according to claim 3, wherein R³ is selected from

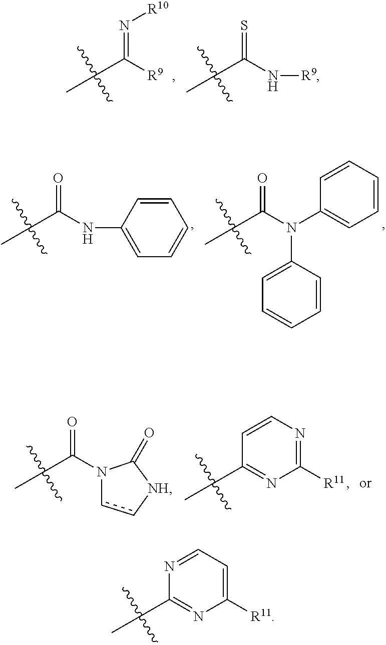

5. The compound according to claim 4, wherein R³ is 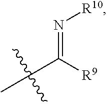
R⁶ is hydrogen, R⁹ is —NHR⁶ and R¹⁰ is —CH₂CH₃.

6-7. (canceled)

8. The compound according to claim 4, wherein R³ is 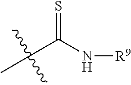
and R⁹ is —CH₃.

9. The compound according to claim 1, wherein X is —CH₂— or —O—.

10-16. (canceled)

17. The compound according to claim 4, wherein R³ is 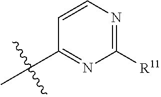
and R¹¹ is —NH₂ or —NHNH₂.

18-19. (canceled)

20. The compound according to claim 4 wherein R³ 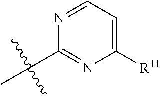
and R¹¹ is hydrogen.

21. The compound according to claim 1, wherein said compound is of formula Ia:

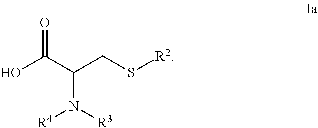

22. The compound according to claim 1, wherein the compound is selected from:

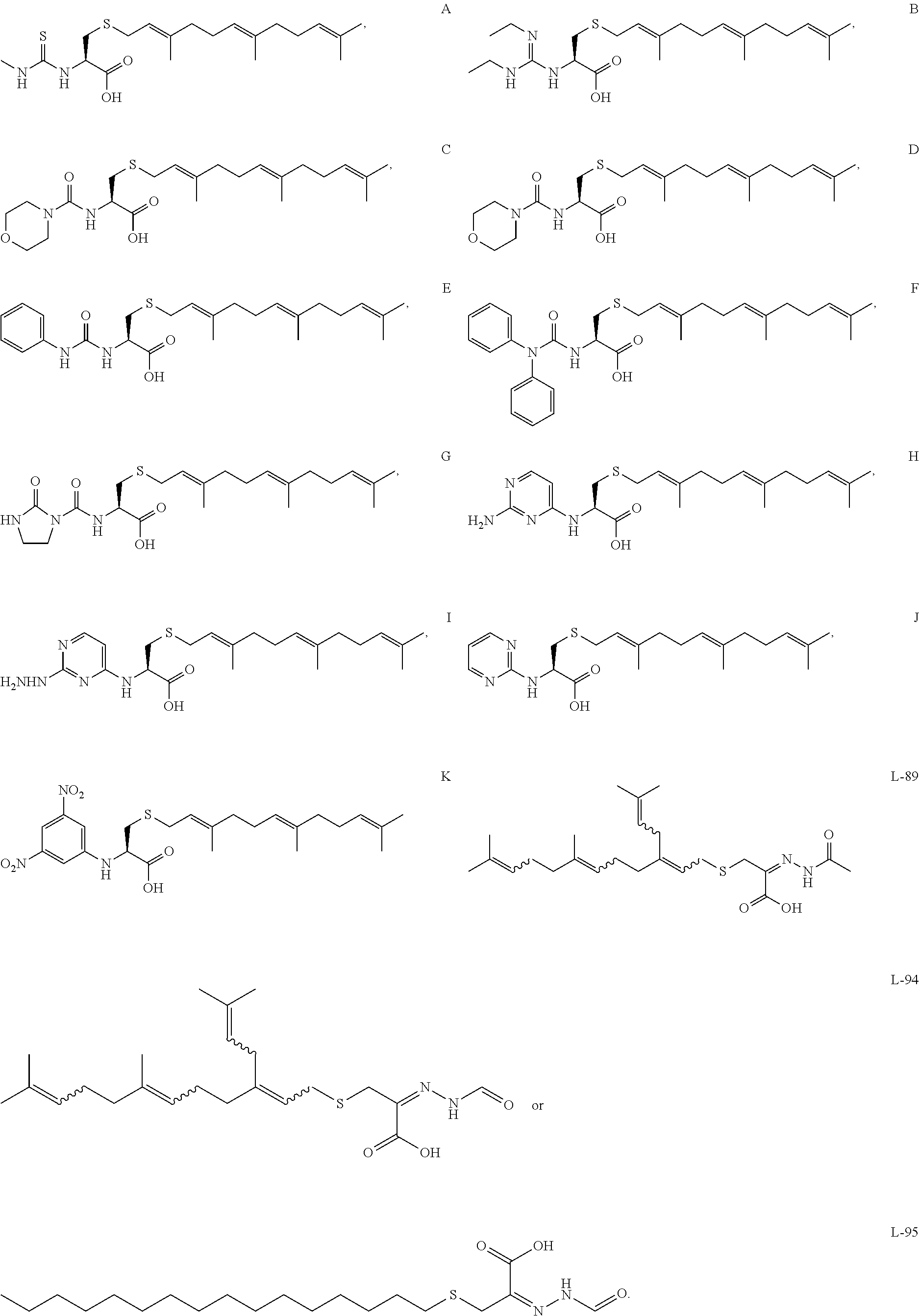

23. (canceled)

24. The compound according to claim 1, wherein the compound is of the structure:

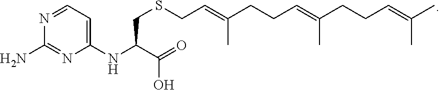

25. A composition comprising a compound according to claim 1, and a pharmaceutically acceptable adjuvant, carrier, or vehicle.

26. The composition according to claim 25, in combination with an additional therapeutic agent.

27. The composition according to claim 26, wherein the additional therapeutic agent is selected from the group consisting of dexamethasone, indomethacin and clobetasol.

28. A method for treating or lessening the severity of an inflammatory disease or disorder in a patient in need thereof, comprising the step of administering to said patient a compound according to claim 1 or a composition thereof, wherein the disease or disorder is selected from inflammation, asthma, an autoimmune disease, COPD, inflammatory responses of the immune system, a skin disease, irritable bowel syndrome, and a neurodegenerative disorder.

29-31. (canceled)

32. The method according to claim 28, wherein the skin disease reduces acute skin irritation.

33. The method according to claim 28, wherein the skin disease is selected from rosacea, atopic dermatitis, seborrheic dermatitis, and psoriasis.

34-35. (canceled)

36. The method according to claim 28, wherein the administering is achieved via transdermal delivery.

37. The method according to claim 28, wherein the administering is in combination with an additional therapeutic agent.

38. The method according to claim 37, wherein the additional therapeutic agent is selected from the group consisting of dexamethasone, indomethacin and clobetasol.

39-41. (canceled)

42. The compound according to claim 1 wherein:
R¹ is 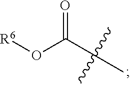
R² is a straight or branched chain aliphatic group having 10 to 25 carbon atoms and one or more double bonds;
R³ is optionally substituted 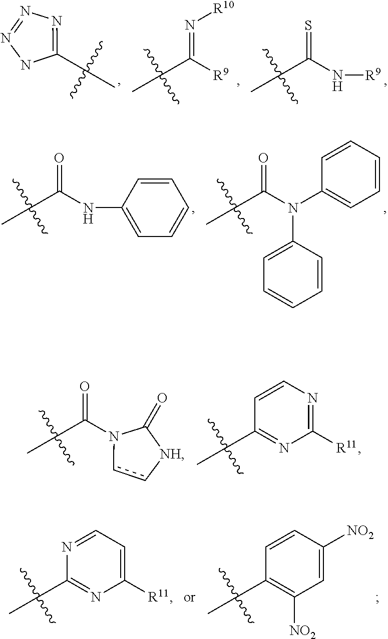
R⁴ is H;
R⁶ is H or alkyl;
R⁹ is H or alkyl;
R¹⁰ is H, alkyl or CN;
R¹¹ is —NH₂, —NHNH₂ or hydrogen;
Z is —S— or —Se—.

43. (canceled)

44. A compound of formula Ib: 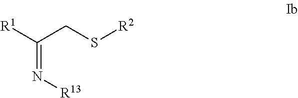
or a pharmaceutically acceptable salt, enantiomer, diastereomer, or double bond isomer thereof, wherein:
R¹ is an optionally substituted heteroaryl group or: 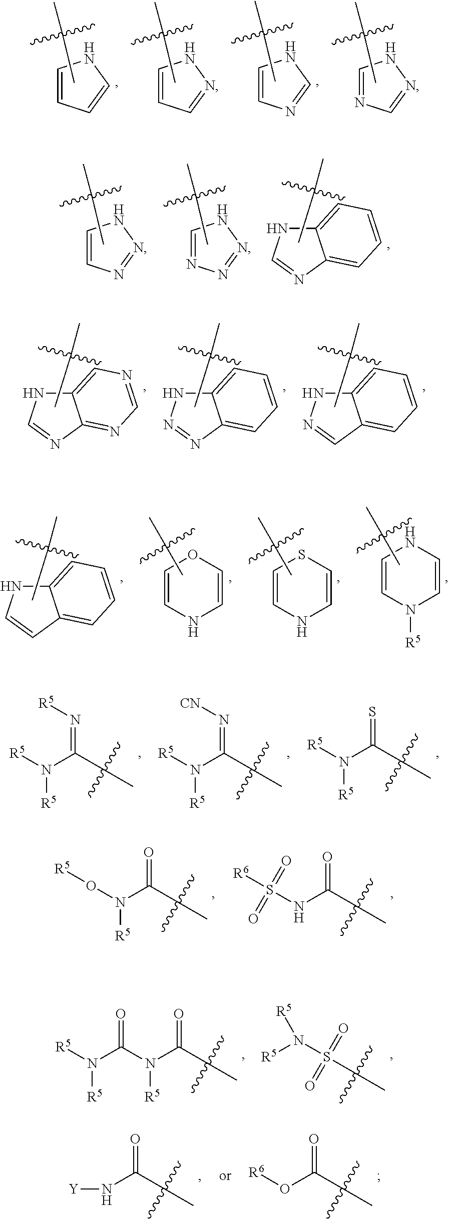
R² is an aliphatic group substituted with one or more R⁷ groups;
R¹³ is independently H 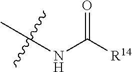
or —NH—S(O)₂R¹⁴;
R¹⁴ is independently H, (C₁-C₄)alkyl or aryl;
R⁵ is independently H, alkyl, aryl, alkenyl, or alkynyl, wherein R⁵ is optionally substituted with one or two R⁷ groups;
R⁶ is H, alkyl, aryl, alkenyl, alkynyl, or a cyclic radical, wherein R⁶ is optionally substituted with one or two R⁷ groups;
R⁷ is —NHC(═O)(C₁-C₈)alkyl, —(C₁-C₈)alkyl, —(C₁-C₈)alkenyl, —(C₁-C₈)alkynyl, phenyl, —(C₂-C₅)heteroaryl, —(C₁-C₆)heterocycloalkyl, —(C₃-C₇)cycloalkyl, —O—(C₁-C₈)alkyl, —O—(C₁-C₈)alkenyl, —O—(C₁-C₈)alkynyl, —O-phenyl, —CN, —OH, oxo, halo, —C(═O)OH, —COhalo, —OC(═O)halo, —CF₃, N₃, NO₂, —NH₂, —NH((C₁-C₈)alkyl), —N((C₁-C₈)alkyl)₂, —NH(phenyl), —N(phenyl)₂, —C(═O)NH₂, —C(═O)NH((C₁-C₈)alkyl), —C(═O)N((C₁-C₈)alkyl)₂, —C(═O)NH(phenyl), —C(═O)N(phenyl)₂, —OC(═O)NH₂, —NHOH, —NOH((C₁-C₈)alkyl), —NOH(phenyl), —OC(═O)NH((C₁-C₈)alkyl), —OC(═O)N((C₁-C₈)alkyl)₂, —OC(═O)NH(phenyl), —OC(═O)N(phenyl)₂, —CHO, —CO((C₁-C₈)alkyl), —CO(phenyl), —C(═O)O((C₁-C₈)alkyl), —C(═O)O(phenyl), —OC(═O)((C₁-C₈)alkyl), —OC(═O)(phenyl), —OC(═O)O((C₁-C₈) alkyl), —OC(═O)O(phenyl), —S—(C₁-C₈)alkyl, —S—(C₁-C₈)alkenyl, —S—(C₁-C₈)alkynyl, and —S-phenyl, —NHS(O)₂-phenyl, —NHS(O)₂-alkyl, —NHS(O)₂—(C₁-C₈)alkenyl, —NHS(O)₂—(C₁-C₈)alkynyl, —NHS(O)₂, —SC(O)-phenyl, —SC(O)-alkyl, —SC(O)—(C₁-C₈)alkenyl, —SC(O)—(C₁-C₈ alkynyl), —O—S(═O)₂—(C₁-C₈)alkyl, —O—S(═O)₂—(C₁-C₈)alkenyl, —O—S(═O)₂—(C₁-C₈)alkynyl, —O—S(═O)₂-phenyl, —(CH₂)ₙNH₂, —(CH₂)ₙ—NH((C₁-C₈)alkyl), —(CH₂)ₙN((C₁-C₈)alkyl)₂, —(CH₂)ₙNH(phenyl), or —(CH₂)ₙN(phenyl)₂, wherein n is 1 to 8.
